SEQUENCE LISTING (4 sequences)

SEQ ID NO: 1 - protein, 218 aa, Homo sapiens
  MISC_FEATURE (127)..(127): X is D or E
  MISC_FEATURE (129)..(129): X is L or M
  MISC_FEATURE (202)..(202): X is A or G
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEXLHNHYTQKSLSLSPGK

SEQ ID NO: 2 - protein, 217 aa, Homo sapiens
  MISC_FEATURE (52)..(52): X is V or M
PAPPVAGPSVFLFPPKPKDTLMISRTPEVTCVVVDVSHEDPEVQFNWYVDGXEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 218 aa, Homo sapiens
PAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2022/0033520 A1
(43) Pub. Date: Feb. 3, 2022

(54) MODIFIED ANTIBODY FCS AND METHODS OF USE

(71) Applicant: Genentech, Inc., South San Francisco, CA (US)

(72) Inventors: Gregory A. Lazar, South San Francisco, CA (US); James Ernst, San Francisco, CA (US); Jasvinder Atwal, San Carlos, CA (US); Shraddha Shirish Sadekar, San Mateo, CA (US); Yanli Yang, Palo Alto, CA (US); Shan Chung, San Mateo, CA (US)

(73) Assignee: Genentech, Inc., South San Francisco, CA (US)

(21) Appl. No.: 17/343,543

(22) Filed: Jun. 9, 2021

Related U.S. Application Data
(63) Continuation of application No. PCT/US2019/067453, filed on Dec. 19, 2019.
(60) Provisional application No. 62/782,904, filed on Dec. 20, 2018.

(51) Int. Cl. C07K 16/40; A61P 25/28; C07K 16/18

(52) U.S. Cl. CPC C07K 16/40; A61P 25/28; C07K 16/18; A61K 2039/505; C07K 2317/92; C07K 2317/52; C07K 2317/77

(57) ABSTRACT

Biological macromolecules have tremendous potential for the treatment of disease of the central nervous system (CNS), however, the presence of the blood brain barrier (BBB) makes achieving a therapeutically relevant antibody concentration extremely challenging. Antibodies with enhanced neutral pH affinity for the neonatal Fc receptor demonstrate improved accumulation in the brain. Variants disclosed herein also enhanced exposure in engineered mouse models. Using an anti-BACE1 antibody, these Fc variants significantly reduced the levels of brain Abeta.

### CROSS-REFERENCE TO RELATED APPLICATION

This application is a continuation of International Patent Application No. PCT/US2019/067453, filed Dec. 19, 2019, which claims the benefit of priority of U.S. Provisional Patent Application No. 62/782,904, filed. Dec. 20, 2018, each of which is incorporated herein by reference in its entirety for any purpose.

### SEQUENCE LISTING

This application contains a Sequence Listing in computer readable form entitled “2019-12-13_01146-0072-00PCT_Seq_List_ST25 4816-1632-8366 v1,” created Dec. 13, 2019, having a size of 8,219 bytes, which is incorporated by reference herein.

### FIELD

The present invention relates to modified Fcs and methods of using modified Fcs to cross the blood-brain barrier.

### BACKGROUND

There are a number of central nervous system (CNS) therapeutic targets with significant medical value—including amyloid beta (Abeta), beta-secretase 1 (BACE1), Tau, and alpha-synuclein (Spillantini, Schmidt et al. 1997, Hardy and Selkoe 2002, Ghosh, Gemma et al. 2008, Thinakaran and Koo 2008, Mandelkow and Mandelkow 2012). The blood-brain barrier (BBB), however, restricts antibody penetration into the brain under normal circumstances (Abbott, Ronnback et al. 2006, Pardridge 2016).

Efforts have been made to improve antibody penetration into the brain by enhanced receptor mediated transcytosis (RMT) (Fishman, Rubin et al. 1987), such as RMT utilizing the transferrin receptor (TfR) (Yu, Zhang et al. 2011, Yu, Atwal et al. 2014, Pardridge 2016). TfR is enriched on brain endothelial cells and is rapidly transcytosed to facilitate transport of transferrin in the brain and other tissues (Ponka and Lok 1999).

Targeting TfR has drawbacks, however. For example, to generate therapeutic antibodies that utilize TfR to cross the BBB, a second binding domain must be introduced to a traditional IgG via one of the numerous multispecific technologies, including, for example, knob-in-hole, dual variable domain, or cross-mAb technologies. These technologies add both expense and complexity to the development of therapeutic antibodies. In addition, TfR is expressed on a number of tissues, and targeting this receptor can introduce a number of potential safety liabilities. For example, targeting TfR may cause a reduction of reticulocytes (Couch, Yu et al. 2013). This risk can be mitigated through the use of various effector-attenuating technologies (Couch, Yu et al. 2013, Lo, Kim et al. 2017). However, as the antibody effector function has been proposed to play a role in the mechanism of action of some Abeta-targeting antibodies, effector attenuation could limit the efficacy of amyloid-targeting antibodies, as well as antibodies targeting other antigens.

Numerous biological antibody receptors can interact with IgG molecules, such as Fc-gamma receptors and the neo-natal Fc receptor (FcRn) (Ghetie and Ward 2002, Challa, Velmurugan et al. 2014). FcRn is a heterodimeric protein complex composed of two subunits—FCGRT, also known as FcRn alpha-chain, and beta-2 microglobulin ((32M). In humans, FcRn is expressed on some hematopoietic cells, kidney cells, gut, and upper airway epithelial cells, as well as on normal endothelial cells, including those located at the BBB (Roopenian and Akilesh 2007, Challa, Velmurugan et al. 2014). During development, FcRn facilitates transport of IgG molecules across the placental barrier, and in infancy FcRn facilitates transport of IgG from milk across gut epithelial cells. In adults, a primary function of FcRn is in mediating endosomal recycling of IgG and consequent persistence of IgG serum half-life. FcRn is expressed in a variety of tissues and cell types, including placenta, liver (including hepatocytes and Kupffer cells), small intestine (including apical enterocytes, goblet cells and enterocytes of crypts), large intestine (including apical enterocytes, goblet cells, enterocytes of crypts, colon, and rectum), oral epithelium, nasopharynx, upper airway (including lung epithelial cells), kidney epithelial cells, endothelial cells, brain endothelial cells, spinal cord, cerebral cortex, choroid plexus, arachnoid villi, bone, lymph node, tonsil, spleen, thyroid, monocytes, macrophages, dendritic cells, B-lymphocytes, NK-cells, adrenal, breast, pancreas, islet of Langerhans, gallbladder, prostate, bladder, skin, uterus, ovary, testes, seminal vesicle, and adipose tissue.

IgG binding to FcRn is regulated by pH. There is virtually no binding of IgG to FcRn at physiological pH, such as in serum, while at acidic pH, such as in endosomes, affinity of IgG to the FcRn is enhanced (Kuo and Aveson 2011). This enhanced binding to FcRn at acidic pH in endosomes results in scavenging of pinocytosed antibodies to prevent lysosomal degradation and to maintain antibody levels in serum (Ghetie and Ward 2002). Research efforts have identified antibody Fc variants having improved pharmacokinetic properties as a result of enhanced binding to the FcRn at acidic pH (Hinton, Johlfs et al. 2004, Dall'Acqua, Kiener et al. 2006, Hinton, Xiong et al. 2006, Petkova, Akilesh et al. 2006, Datta-Mannan, Witcher et al. 2007, Yeung, Leabman et al. 2009, Zalevsky, Chamberlain et al. 2010). Some Fc variants have been identified that improve binding to the FcRn at both physiological and acidic pH (Hinton, Johlfs et al. 2004, Dall'Acqua, Kiener et al. 2006, Hinton, Xiong et al. 2006, Yeung, Leabman et al. 2009, Igawa, Maeda et al. 2013). However, several of these Fc variants can also result in enhanced antibody clearance (Dall'Acqua, Woods et al. 2002, Vaccaro, Zhou et al. 2005, Igawa, Maeda et al. 2013, Borrok, Wu et al. 2015).

Previous research suggested that the FcRn has limited function in transport of antibodies into the brain (Abuqayyas and Balthasar 2013), and indeed, experiments with direct intra-cranial antibody injection showed that the FcRn functions to facilitate export of antibodies out of the brain (Cooper, Ciambrone et al. 2013).

### SUMMARY

Provided herein are antibodies and Fc conjugates comprising modified Fcs and having improved brain uptake.

In some embodiments, methods of treating a neurological disorder in a subject are provided, comprising administering an antibody comprising a modified IgG Fc to a subject in need thereof, wherein the antibody is active in an in vitro transcytosis assay. In some embodiments, the neurological disorder is selected from a neuropathy disorder, a neurodegenerative disease, a brain disorder, cancer, an ocular disease disorder, a seizure disorder, a lysosomal storage disease, amyloidosis, a viral or microbial disease, ischemia, a behavioral disorder, and CNS inflammation. In some embodiments, the neurological disorder is a neurodegenerative disease. In some embodiments, the neurodegenerative disease is selected from Lewy body disease, postpoliomyelitis syndrome, Shy-Draeger syndrome, olivopontocerebellar atrophy, amyloidosis, Parkinson's disease, multiple system atrophy, striatonigral degeneration, an amyloidosis, a tauopathy, Alzheimer disease, supranuclear palsy, prion diseases, bovine spongiform encephalopathy, scrapie, Creutzfeldt-Jakob syndrome, kuru, Gerstmann-Straussler-Scheinker disease, chronic wasting disease, and fatal familial insomnia.

In some embodiments, methods of delivering an antibody to the brain of a subject are provided, comprising administering to the subject an antibody comprising a modified IgG Fc to a subject in need thereof, wherein the antibody is active in an in vitro transcytosis assay.

In some embodiments, methods of increasing brain exposure to an antibody are provided, comprising administering to a subject an antibody comprising a modified IgG Fc to a subject in need thereof, wherein the antibody is active in an in vitro transcytosis assay.

In some embodiments, methods of increasing transport of an antibody across the blood brain barrier (BBB) are provided, comprising administering to a subject an antibody comprising a modified IgG Fc to a subject in need thereof, wherein the antibody is active in an in vitro transcytosis assay.

In some embodiments, an isolated antibody is provided, wherein the antibody comprises a modified IgG Fc, wherein the antibody is active in an in vitro transcytosis assay.

In some embodiments, the antibody exhibits a transcytosis activity in the in vitro transcytosis assay of at least 50 when normalized to the same antibody comprising a wild-type IgG Fc. In some embodiments, the antibody exhibits a transcytosis activity in the in vitro transcytosis assay of at least 60, at least 70, at least 80, at least 90, or at least 100. In some embodiments, the in vitro transcytosis assay comprises cells that express FcRn. In some embodiments, the FcRn is human FcRn. In some embodiments, the cells are MDCK II cells.

In some embodiments, the antibody comprising the modified IgG Fc has a binding affinity for FcRn at pH 7.4 that is greater than the binding affinity of a reference antibody with an unmodified IgG Fc of the same species and isotype. In some embodiments, the antibody comprising the modified IgG Fc has a binding affinity for FcRn at pH 6 that is greater than the binding affinity of a reference antibody with an unmodified IgG Fc of the same species and isotype. In some embodiments, the antibody comprising the modified IgG Fc has a binding affinity for FcRn at pH 7.4 of ≤10 μM, ≤5 μM, ≤4 μM, ≤3 μM, ≤2 μM, ≤1 μM, ≤900 nM, ≤800 nM, ≤700 nM, ≤600 nM, ≤500 nM, ≤400 nM, ≤300 nM, ≤200 nM, or ≤100 nM. In some embodiments, the antibody comprising the modified IgG Fc has a binding affinity for FcRn at pH 6 of ≤1 μM, ≤900 nM, ≤800 nM, ≤700 nM, ≤600 nM, ≤500 nM, ≤400 nM, ≤300 nM, ≤200 nM, ≤100 nM, ≤90 nM, ≤80 nM, ≤70 nM, ≤60 nM, ≤50 nM, ≤40 nM, ≤30 nM, ≤20 nM, or ≤10 nM. In some embodiments, the ratio of the affinity of the antibody comprising the modified IgG Fc for FcRn at pH 7.4 to the affinity of the antibody comprising the modified IgG Fc for FcRn at pH 6 is at least 5, at least 10, at least 20, at least 50, or at least 100; or 5 to 200, 5 to 100, 10 to 200, 10 to 100, 20 to 100, or 20 to 200.

In some embodiments, the antibody comprising the modified IgG Fc comprises one or more mutations selected from 252W, 252Y, 286E, 286Q, 307Q, 308P, 310A, 311A, 311I, 428L, 433K, 434F, 434W, 434Y, and 436I by EU numbering. In some embodiments, the modified IgG Fc comprises 252Y and 434Y. In some embodiments, the modified IgG Fc comprises 252Y and 434Y and one or two additional mutations selected from 286E, 286Q, 307Q, 308P, 311A, 311I, 428L, 433K, and 436I. In some embodiments, the modified IgG Fc further comprises 307Q and 311A, or further comprises 286E. In some embodiments, the modified IgG Fc comprises a set of mutations selected from the sets of mutations in Tables 4, 5, and 6. In some embodiments, the modified IgG Fc comprises one or more modifications of a sequence selected from SEQ ID NOs: 1-4. In some embodiments, the IgG Fc is an IgG1 Fc. In some embodiments, the IgG Fc is an IgG4 Fc.

In some embodiments, the antibody binds to a brain antigen. In some embodiments, the antibody binds to a brain antigen selected from beta-secretase 1 (BACE1), amyloid beta (Abeta), epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), interleukin 6 receptor (IL6R), interleukin 1 beta (IL1β), caspase 6, triggering receptor expressed on myeloid cells 2 (TREM2), C1q, paired immunoglobin like type 2 receptor alpha (PILRA), CD33, interleukin 6 (IL6), tumor necrosis factor alpha (TNFα), tumor necrosis factor receptor superfamily member 1A (TNFR1), tumor necrosis factor receptor superfamily member 1B (TNFR2), and apolipoprotein J (ApoJ).

In some embodiments, the antibody is a monoclonal antibody. In some embodiments, the antibody is a human, humanized, or chimeric antibody. In some embodiments, the antibody is a bispecific antibody. In some embodiments, the antibody is an antibody fragment.

In some embodiments, the antibody is conjugated to an imaging agent. In some embodiments, the antibody is conjugated to a neurological disorder drug. In some embodiments, the neurological disorder drug is selected from an aptamer, an inhibitory nucleic acid, a ribozyme, and a small molecule.

In some embodiments, methods of treating a neurological disorder are provided, comprising administering an Fc conjugate comprising a modified IgG Fc to a subject in need thereof, wherein the Fc conjugate is active in an in vitro transcytosis assay. In some embodiments, the neurological disorder is selected from a neuropathy disorder, a neurodegenerative disease, cancer, an ocular disease disorder, a seizure disorder, a lysosomal storage disease, amyloidosis, a viral or microbial disease, ischemia, a behavioral disorder, and CNS inflammation. In some embodiments, the neurological disorder is a neurodegenerative disease. In some embodiments, the neurodegenerative disease is selected from Lewy body disease, postpoliomyelitis syndrome, Shy-Draeger syndrome, olivopontocerebellar atrophy, Parkinson's disease, multiple system atrophy, striatonigral degeneration, a tauopathy, Alzheimer disease, supranuclear palsy, prion diseases, bovine spongiform encephalopathy, scrapie, Creutzfeldt-Jakob syndrome, kuru, Gerstmann-Straussler-Scheinker disease, chronic wasting disease, and fatal familial insomnia.

In some embodiments, methods of delivering an Fc conjugate to the brain of a subject are provided, comprising administering to the subject an antibody comprising a modified IgG Fc to a subject in need thereof, wherein the Fc conjugate is active in an in vitro transcytosis assay.

In some embodiments, methods of increasing brain exposure to an Fc conjugate are provided, comprising administering to a subject an antibody comprising a modified IgG Fc to a subject in need thereof, wherein the Fc conjugate is active in an in vitro transcytosis assay.

In some embodiments, methods of increasing transport of an Fc conjugate across the blood brain barrier (BBB) are provided, comprising administering to a subject an antibody comprising a modified IgG Fc to a subject in need thereof, wherein the Fc conjugate is active in an in vitro transcytosis assay.

In some embodiments, an Fc conjugate is provided, wherein the Fc conjugate comprising a modified IgG Fc, wherein the Fc conjugate is active in an in vitro transcytosis assay.

In some embodiments, the Fc conjugate exhibits a transcytosis activity in the in vitro transcytosis assay of at least 50 when normalized to the same Fc conjugate comprising a wild-type IgG Fc. In some embodiments, the Fc conjugate exhibits a transcytosis activity in the in vitro transcytosis assay of at least 60, at least 70, at least 80, at least 90, or at least 100. In some embodiments, the in vitro transcytosis assay comprises cells that express FcRn. In some embodiments, the FcRn is human FcRn. In some embodiments, the cells are MDCK II cells.

In some embodiments, the Fc conjugate comprising the modified IgG Fc has a binding affinity for FcRn at pH 7.4 that is greater than the binding affinity of a reference Fc conjugate with an unmodified IgG Fc of the same species and isotype. In some embodiments, the Fc conjugate comprising the modified IgG Fc has a binding affinity for FcRn at pH 6 that is greater than the binding affinity of a reference Fc conjugate with an unmodified IgG Fc of the same species and isotype. In some embodiments, the Fc conjugate comprising the modified IgG Fc has a binding affinity for FcRn at pH 7.4 of less than 1 mM, or less than 750 nM, or less than or less than 500 nM, or less than 400 nM, or less than 300 nM, or less than 200 nM, or less than 100 nM, or between 50 nM and 1 mM, or between 100 nM and 1 mM, or between 100 nM and 500 nM. In some embodiments, the Fc conjugate comprising the modified IgG Fc has a binding affinity for FcRn at pH 6 of less than 100 nM, or less than 90 nM, or less than 80 nM, or less than 70 nM, or less than 60 nM, or less than 50 nM, or less than 40 nM, or less than 30 nM, or less than 20 nM, or less than 10 nM, or between 1 nM and 200 nM, or between 10 nM and 200 nM, or between 10 nM and 100 nM. In some embodiments, the ratio of the affinity of the Fc conjugate comprising the modified IgG Fc for FcRn at pH 7.4 to the affinity of the Fc conjugate comprising the modified IgG Fc for FcRn at pH 6 is at least 5, at least 10, at least 20, at least 50, or at least 100; or 5 to 200, 5 to 100, 10 to 200, 10 to 100, 20 to 100, or 20 to 200.

In some embodiments, the modified IgG Fc comprises one or more mutations selected from 252W, 252Y, 286E, 286Q, 307Q, 308P, 310A, 311A, 311I, 428L, 433K, 434F, 434W, 434Y, and 436I by EU numbering. In some embodiments, the modified IgG Fc comprises 252Y and 434Y. In some embodiments, the modified IgG Fc comprises 252Y and 434Y and one or two additional mutations selected from 286E, 286Q, 307Q, 308P, 311A, 311I, 428L, 433K, and 436I. In some embodiments, the modified IgG Fc further comprises 307Q and 311A, or further comprises 286E. In some embodiments, the modified IgG Fc comprises a set of mutations selected from the sets of mutations in Tables 4, 5, and 6. In some embodiments, the modified IgG Fc comprises one or more modifications of a sequence selected from SEQ ID NOs: 1-4. In some embodiments, the IgG Fc is an IgG1 Fc. In some embodiments, the IgG Fc is an IgG4 Fc.

In some embodiments, the Fc conjugate comprises the modified IgG Fc fused to a therapeutic protein. In some embodiments, the therapeutic protein is selected from a receptor extracellular domain and an enzyme. In some embodiments, the receptor extracellular domain is selected from a TNF-R1 extracellular domain (ECD), a CTLA-4 ECD, and an IL-1R1 ECD. In some embodiments, the enzyme is selected from alpha-L-iduronidase, iduronate-2-sulphatase, N-sulfatase, alpha-N-acetylglucosaminidase, N-acetyl-galactosamine-6-sulfatase, beta-galactosidase, arylsulphatase B, beta-glucuronidase, acid alpha-glucosidase, glucocerebrosidase, alpha-galactosidase A, hexosaminidase A, acid sphingomyelinase, beta-galactocerebrosidase, beta-galactosidase, arylsulfatase A, acid ceramidase, aspartoacylase, palmitoyl-protein thioesterase 1, and tripeptidyl amino peptidase 1.

In some embodiments, the Fc conjugate comprises the modified IgG Fc conjugated to a neurological disorder drug. In some embodiments, the neurological disorder drug is selected from an aptamer, an inhibitory nucleic acid, a ribozyme, and a small molecule. In some embodiments, the Fc conjugate comprises the modified IgG Fc conjugated to an imaging agent.

### BRIEF DESCRIPTION OF THE FIGURES

FIGS. 1A-1D. Pharmacokinetics and pharmacodynamics of an anti-BACE1 hIgG1 antibody (anti-BACE1), an anti-BACE1 hIgG1 antibody with a modified Fc (anti-BACE1-YTE), or a control anti-gD hIgG1 antibody (anti-gD) in wild-type mice. A) Plasma antibody pharmacokinetics; B) Brain pharmacodynamics, as measured by brain Abeta levels; C) Brain antibody pharmacokinetics. D) Relative antibody uptake in brain of the human IgG1 YTE modified antibody compared to wild-type IgG1, including maximum concentration (Cmax) and area under the curve (AUC).

FIGS. 2A-2D. Pharmacokinetics and pharmacodynamics of anti-BACE1 hIgG1 antibody (anti-BACE1), an anti-BACE1 hIgG1 antibody with a modified Fc (anti-BACE1-YTE), or anti-gD hIgG1 antibody in transgenic (Tg32) mice comprising human FCGRT (hFcRn alpha-chain) and lacking murine Fcgrt (i.e., FCGRT^{+/+} Fcgrt^{-/-} mice), and which express hFCGRT and lack mFCGRT. A) Plasma antibody pharmacokinetics; B) Brain pharmacodynamics, measured by brain Abeta levels; C) Brain antibody pharmacokinetics; D) In vitro human and murine FcRn binding properties of a human IgG1 YTE modified antibody or a human IgG1 wild type antibody.

FIG. 3. Plot of binding affinity of certain hIgG1 antibodies comprising mutations in the Fc to hFcRn at two different pHs, as described in Example 3. The X-axis shows the pH6 affinities of the antibodies, while the Y-axis shows the pH 7.4 affinities of the antibodies.

FIG. 4. Graph of normalized transcytosis values of certain Fc modified antibodies, as described in Example 3. Fc modified antibodies above the dashed line showed significantly improved transcytosis.

FIGS. 5A-5C. Improved brain exposure properties of anti-gD hIgG1 antibodies comprising modified Fcs in transgenic (Tg32) mice, which express hFCGRT and lack mFCGRT. A) Serum pharmacokinetics (PK); B) Brain PK; C) Summary of single-dose PK data and affinity data.

FIGS. 6A-6E. Pharmacokinetics and pharmacodynamics of an anti-BACE1 hIgG1 antibody with a modified Fc (anti-BACE1-YQAY) in transgenic (Tg32) mice, which express hFCGRT and lack mFCGRT. A) Serum antibody concentration; B) Brain pharmacokinetics (PK); C) Brain pharmacodynamics (PD), measured by brain Abeta levels; D) Summary of hFcRn affinities and brain PK data; E) Summary of brain PD data.

FIGS. 7A-7D. Plasma pharmacokinetics and binding affinity of D1 (YY) and Q95 (YQAY) hIgG1 modified Fc antibodies administered to transgenic Tg32 (FCGRT^{+/+} Fcgrt^{-/-}) mice, which express hFCGRT and lack mFCGRT; hemizygous (FCGRT^{+/-} Fcgrt^{+/-}) mice, which express both hFCGRT and mFCGRT; or wild-type (Fcgrt^{+/+}) mice, which express only mFCGRT. A) Plasma PK in homozygous Tg32 mice; B) Plasma PK in hemizygous Tg32 (FCGRT^{+/-} Fcgrt^{+/-}) mice; C) Plasma PK in wildtype (Fcgrt^{+/+}) mice; D) Summary of FcRn binding affinity data and ratio of plasma AUC of each FC modified antibody relative to wild-type antibody.

FIGS. 8A-8C. Brain pharmacokinetics and brain antibody concentration of anti-gD hIgG1 antibodies comprising modified Fcs in transgenic Tg32 mice, which express hFCGRT and lack mFCGRT. A) Brain PK data; B) Brain antibody concentrations at 7 days post-dose; C) Summary of affinity data and ratio of brain AUC of each Fc modified antibody relative to wild-type antibody.

FIGS. 9A-9C. Liver exposure of anti-gD hIgG1 antibodies comprising modified Fcs in transgenic Tg32 mice, which express hFCGRT and lack mFCGRT. A) Liver PK data; B) Liver antibody concentrations at 7 days post-dose; C) Summary of affinity data and ratio of liver AUC of each Fc modified antibody relative to wild-type antibody.

FIGS. 10A-10C. Large intestine exposure of anti-gD hIgG1 antibodies comprising modified Fcs in transgenic Tg32 mice, which express hFCGRT and lack mFCGRT. A) Large intestine PK data; B) Large intestine antibody concentrations at 7 days post-dose; C) Summary of affinity data and ratio of large intestine AUC of each Fc modified antibody relative to wild-type antibody.

FIGS. 11A-11C. Lung exposure of anti-gD hIgG1 antibodies comprising modified Fcs in transgenic Tg32 mice, which express hFCGRT and lack mFCGRT. A) Lung PK data; B) Lung antibody concentrations at 7 days post-dose; C) Summary of affinity data and ratio of lung AUC of each Fc modified antibody relative to wild-type antibody.

FIG. 12. Aligned sequences of human IgG subclasses IgG1, IgG2, IgG3, and IgG4 (SEQ ID NOs: 1-4, respectively). Differences in sequence from IgG1 are highlighted in gray, and the presence of two residues separated by a slash indicates common polymorphic variants. Positions are numbered according to the EU index as described in Kabat (SEQUENCES OF IMMUNOLOGICAL INTEREST, 5th edition, NIH publication, No. 91-3242, E. A. Kabat et al.). The EU index or EU numbering scheme refers to the numbering of the EU antibody as described in Edelman et al., 1969, Proc Natl Acad Sci USA 63:78-85.

FIGS. 13A-13D. Pharmacokinetics and pharmacodynamics of an anti-BACE1 antibody with modified Fcs (anti-BACE1-YQAY and anti-BACE1-YY) in transgenic (Tg32) mice, which express hFCGRT and lack mFCGRT. A) Serum antibody concentration; B) Brain pharmacokinetics (PK); C) Brain pharmacodynamics (PD), measured by brain Abeta levels; D) Summary of hFcRn affinities, brain PK data, and brain PD data.

FIGS. 14A-14C. Pharmacokinetics and pharmacodynamics of an anti-BACE1 hIgG1 antibody (anti-BACE1 WT) and anti-BACE1 hIgG1 antibodies with modified Fcs (anti-BACE1-YEY, YQAY, YPY) in cynomolgus monkeys. A) CNS pharmacodynamics, as measured by sAPPβ/α ratio in CSF at various times following antibody administration. B) Serum antibody concentration at various times following antibody administration. C) Antibody serum exposure from day 0 to day 7, based on data in (B).

FIGS. 15A-15J. Pharmacokinetics and pharmacodynamics of an anti-BACE1 hIgG1 antibody (anti-BACE1 WT, or “WT”) and anti-BACE1 hIgG1 antibodies with modified Fcs (anti-BACE1-YQAY, YY, YLYI, YIY) in cynomolgus monkeys. A) CNS pharmacodynamics, as measured by sAPPβ/α ratio in CSF at various times following antibody administration. B) Brain pharmacodynamics as measured by sAPPβ/α ratio from brain tissue on day 2 and day 7. C) CSF versus brain pharmacodynamics as measured by the ratio of sAPPβ/α on day 2 and day 7. D) Brain antibody concentrations (cortex and hippocampus) at day 2 and day 7 following antibody administration. E) Fold change in brain antibody concentrations, compared to hIgG1 wild-type at day 2 and day 7, based on the data in (B). F) The correlation of brain pharmacokinetics to brain pharmacodynamics. G) CSF antibody concentration at various times following antibody administration. H) Ratio of antibody concentrations in CSF and serum at various times following antibody administration. I) Serum antibody concentration at various times following antibody administration. J) Antibody serum exposure, based on the data in (I).

FIGS. 16A-16F. Concentrations of an anti-Abeta hIgG4 antibody and an anti-Abeta hIgG4 antibody with a modified Fc (anti-Abeta hIgG4-YTE) in plasma (A) and cerebellum (B) following administration to PS2APP mice expressing mFCGRT at the indicated dose. C) Anti-Abeta hIgG4 (“Abeta”), anti-Abeta hIgG4 YTE (“Abeta YTE”), and anti-gD hIgG4 binding to oligomeric Abeta in the mossy fiber hippocampal tract of PS2APP mice following administration to mice at the indicated dose. D) Bar graph of the staining levels observed in (C). Binding of anti-Abeta hIgG4 and anti-Abeta hIgG4 YTE to target Abeta plaques in the subiculum (E) and prefrontal cortex (F) of PS2APP mice.

FIGS. 17A-17F. A) Average brain concentration of anti-Abeta hIgG4 wild-type (“WT”) and anti-Abeta hIgG4 YY, YQAY, and YEY antibodies on days 2 and 7 following a single administration to cynomolgus monkeys. B) Fold change in brain antibody concentrations, compared to hIgG4 wild-type at day 2 and day 7, based on the data in (A). C) CSF concentration of anti-Abeta hIgG4 wild-type (“WT”) and anti-Abeta hIgG4 YY, YQAY, and YEY antibodies following a single administration to cynomolgus monkeys. D) Ratio of antibody concentrations in CSF and serum following a single administration of anti-Abeta hIgG4 wild-type (“WT”) and anti-Abeta hIgG4 YY, YQAY, and YEY to cynomolgus monkeys. E) Serum pharmacokinetics of anti-Abeta hIgG4 wild-type (“WT”) antibody and anti-Abeta hIgG4 antibodies with modified Fcs (YY, YQAY, and YEY) following administration to cynomolgus monkeys. F) Antibody serum exposure, based on the data in (E).

FIG. 18A-18B. A) Correlation between antibody serum exposure and antibody affinity at pH7.4 for both anti-BACE1 hIgG1 and anti-Abeta hIgG4 antibodies with modified Fc. B) Correlation between antibody partitioning to brain relative to serum and antibody affinity at pH7.4 for both anti-BACE1 hIgG1 and anti-Abeta hIgG4 antibodies with modified Fc.

### DETAILED DESCRIPTION OF CERTAIN EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

Unless defined otherwise, technical and scientific terms used herein have the same meaning as commonly understood by one of ordinary skill in the art to which this invention belongs, and are consistent with: Singleton et al (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

When trade names are used herein, applicants intend to independently include the trade name product formulation, the generic drug, and the active pharmaceutical ingredient(s) of the trade name product.

### Definitions

Unless stated otherwise, the following terms and phrases as used herein are intended to have the following meanings:

“Affinity” refers to the strength of the sum total of noncovalent interactions between a single binding site of a molecule and its binding partner. Unless indicated otherwise, as used herein, “binding affinity” refers to intrinsic binding affinity which reflects a 1:1 interaction between members of a binding pair (e.g., antibody and antigen, or IgG constant region or Fc and FcRn). The affinity of a molecule X for its partner Y can generally be represented by the equilibrium dissociation constant (K_{D}, which is a ratio of the off-rate of X from Y (kd or koff) to the on-rate of X to Y (ka or kon)). A surrogate measurement for the affinity of one or more antibodies for its target is its half maximal inhibitory concentration (IC50), a measure of how much of the antibody is needed to inhibit the binding of a known ligand to the antibody target by 50%. Affinity can be measured by common methods known in the art, including those described herein. Specific illustrative and exemplary embodiments for measuring binding affinity are described herein.

The “blood-brain barrier” or “BBB” refers to the physiological barrier between the peripheral circulation and the brain and spinal cord (i.e., the CNS) which is formed by tight junctions within the brain capillary endothelial plasma membranes, creating a tight barrier that restricts the transport of molecules into the brain, even very small molecules such as urea (60 Daltons). The blood-brain barrier within the brain, the blood-spinal cord barrier within the spinal cord, and the blood-retinal barrier within the retina are contiguous capillary barriers within the CNS, and are herein collectively referred to the blood-brain barrier or BBB. The BBB also encompasses the blood-CSF barrier (choroid plexus) where the barrier is comprised of ependymal cells rather than capillary endothelial cells.

The terms “amyloid beta,” “beta-amyloid,” “Abeta,” “amyloidβ,” and “Aβ”, used interchangeably herein, refer to the fragment of amyloid precursor protein (“APP”) that is produced upon β-secretase 1 (“BACE1”) and γ-secretase cleavage of APP, as well as modifications, fragments and any functional equivalents thereof, including, but not limited to, Aβ1-40, and Aβ1-42. Aβ is known to exist in monomeric form, as well as to associate to form oligomers and fibril structures, which may be found as constituent members of amyloid plaque. The structure and sequences of such Aβ peptides are well known to one of ordinary skill in the art and methods of producing said peptides or of extracting them from brain and other tissues are described, for example, in Glenner and Wong, Biochem Biophys Res. Comm. 129: 885-890 (1984). Moreover, Aβ peptides are also commercially available in various forms.

“Anti-Abeta immunoglobulin,” “anti-Abeta antibody,” and “antibody that binds Abeta” are used interchangeably herein, and refer to an antibody that specifically binds to human Abeta. A nonlimiting example of an anti-Abeta antibody is crenezumab. Other non-limiting examples of anti-Abeta antibodies are solanezumab, bapineuzumab, gantenerumab, aducanumab, ponezumab and any anti-Abeta antibodies disclosed in the following publications: WO2000162801, WO2002046237, WO2002003911, WO2003016466, WO2003016467, WO2003077858, WO2004029629, WO2004032868, WO2004032868, WO2004108895, WO2005028511, WO2006039470, WO2006036291, WO2006066089, WO2006066171, WO2006066049, WO2006095041, WO2009027105.

The terms “crenezumab” and “MABT5102A” are used interchangeably herein, and refer to a specific anti-Abeta antibody that binds to monomeric, oligomeric, and fibril forms of Abeta, and which is associated with CAS registry number 1095207.

The term “amyloidosis,” as used herein, refers to a group of diseases and disorders caused by or associated with amyloid or amyloid-like proteins and includes, but is not limited to, diseases and disorders caused by the presence or activity of amyloid-like proteins in monomeric, fibril, or polymeric state, or any combination of the three, including by amyloid plaques. Such diseases include, but are not limited to, secondary amyloidosis and age-related amyloidosis, such as diseases including, but not limited to, neurological disorders such as Alzheimer's Disease (“AD”), diseases or conditions characterized by a loss of cognitive memory capacity such as, for example, mild cognitive impairment (MCI), Lewy body dementia, Down's syndrome, hereditary cerebral hemorrhage with amyloidosis (Dutch type), the Guam Parkinson-Dementia complex and other diseases which are based on or associated with amyloid-like proteins such as progressive supranuclear palsy, multiple sclerosis, Creutzfeld Jacob disease, Parkinson's disease, HIV-related dementia, ALS (amyotropic lateral sclerosis), inclusion-body myositis (IBM), adult onset diabetes, endocrine tumor and senile cardiac amyloidosis, and various eye diseases including macular degeneration, drusen-related optic neuropathy, glaucoma, and cataract due to beta-amyloid deposition.

Glaucoma is a group of diseases of the optic nerve involving loss of retinal ganglion cells (RGCs) in a characteristic pattern of optic neuropathy. RGCs are the nerve cells that transmit visual signals from the eye to the brain. Caspase-3 and Caspase-8, two major enzymes in the apoptotic process, are activated in the process leading to apoptosis of RGCs. Caspase-3 cleaves amyloid precursor protein (APP) to produce neurotoxic fragments, including Abeta. Without the protective effect of APP, Abeta accumulation in the retinal ganglion cell layer results in the death of RGCs and irreversible loss of vision.

Glaucoma is often, but not always, accompanied by an increased eye pressure, which may be a result of blockage of the circulation of aqueous, or its drainage. Although raised intraocular pressure is a significant risk factor for developing glaucoma, no threshold of intraocular pressure can be defined which would be determinative for causing glaucoma. The damage may also be caused by poor blood supply to the vital optic nerve fibers, a weakness in the structure of the nerve, and/or a problem in the health of the nerve fibers themselves. Untreated glaucoma leads to permanent damage of the optic nerve and resultant visual field loss, which can progress to blindness.

The “central nervous system” or “CNS” refers to the complex of nerve tissues that control bodily function, and includes the brain and spinal cord.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS, including brain metastases resulting from cancer elsewhere in the body).

A “neurological disorder drug” is a drug or therapeutic agent that treats one or more neurological disorder(s). Neurological disorder drugs of the invention include, but are not limited to, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, and small molecules, or active fragments of any of the foregoing. Exemplary neurological disorder drugs of the invention are described herein and include, but are not limited to: antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins. Non-limiting examples of neurological disorder drugs and the disorders they may be used to treat are provided in the following Table 1:

TABLE 1
Non-limiting examples of neurological disorder drugs and
the corresponding disorders they may be used to treat

| Drug | Neurological disorder |
|---|---|
| Anti-BACE1 Antibody | Alzheimer's, acute and |
| | chronic brain injury, stroke |
| Anti-Abeta Antibody | Alzheimer's disease, amyloidoses |
| Anti-Tau Antibody | Alzheimer's disease, tauopathies |
| Neurotrophin | Stroke, acute brain injury, |
| | spinal cord injury |
| Brain-derived neurotrophic | Chronic brain injury |
| factor (BDNF), Fibroblast | (Neurogenesis) |
| growth factor 2 (FGF-2) | |
| Anti-Epidermal Growth | Brain cancer |
| Factor Receptor (EGFR)- | |
| antibody | |
| Glial cell-line derived | Parkinson's disease |
| neural factor (GDNF) | |
| Brain-derived neurotrophic | Amyotrophic lateral |
| factor (BDNF) | sclerosis, depression |
| Lysosomal enzyme | Lysosomal storage disorders |
| | of the brain |
| Ciliary neurotrophic | Amyotrophic lateral sclerosis |
| factor (CNTF) | |
| Neuregulin-1 | Schizophrenia |
| Anti-HER2 antibody (e.g. | Brain metastasis from HER2- |
| trastuzumab, pertuzumab, | positive |
| etc.) | cancer |
| Anti-VEGF antibody (e.g., | Recurrent or newly diagnosed |
| bevacizumab) | glioblastoma, recurrent |
| | malignant glioma, brain |
| | metastasis |

An “imaging agent” is a compound that has one or more properties that permit its presence and/or location to be detected directly or indirectly. Examples of such imaging agents include proteins and small molecule compounds incorporating a labeled moiety that permits detection.

A “CNS antigen” or “brain antigen” is an antigen expressed in the CNS, including the brain, which can be targeted with an antibody or small molecule. Examples of such antigens include, without limitation: beta-secretase 1 (BACE1), amyloid beta (Abeta), epidermal growth factor receptor (EGFR), human epidermal growth factor receptor 2 (HER2), tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), interleukin 6 receptor (IL6R), interleukin 1 beta (IL1β), caspase 6, triggering receptor expressed on myeloid cells 2 (TREM2), C1q, paired immunoglobin like type 2 receptor alpha (PILRA), CD33, interleukin 6 (IL6), tumor necrosis factor alpha (TNFα), tumor necrosis factor receptor superfamily member 1A (TNFR1), tumor necrosis factor receptor superfamily member 1B (TNFR2), and apolipoprotein J (ApoJ). In one embodiment, the antigen is BACE1.

The term “BACE1,” as used herein, refers to any native beta-secretase 1 (also called β-site amyloid precursor protein cleaving enzyme 1, membrane-associated aspartic protease 2, memapsin 2, aspartyl protease 2 or Asp2) from any vertebrate source, including mammals such as primates (e.g. humans) and rodents (e.g., mice and rats), unless otherwise indicated. The term encompasses “full-length,” unprocessed BACE1 as well as any form of BACE1 which results from processing in the cell. The term also encompasses naturally occurring variants of BACE1, e.g., splice variants or allelic variants. The amino acid sequence of an exemplary BACE1 polypeptide is the sequence for human BACE1, isoform A as reported in Vassar et al., Science 286:735-741 (1999), which is incorporated herein by reference in its entirety. Several other isoforms of human BACE1 exist including isoforms B, C and D. See UniProtKB/Swiss-Prot Entry P56817, which is incorporated herein by reference in its entirety.

The terms “anti-beta-secretase antibody”, “anti-BACE1 antibody”, “an antibody that binds to beta-secretase” and “an antibody that binds to BACE1” refer to an antibody that is capable of binding BACE1 with sufficient affinity such that the antibody is useful as a diagnostic and/or therapeutic agent in targeting BACE1. In one embodiment, the extent of binding of an anti-BACE1 antibody to an unrelated, non-BACE1 protein is less than about 10% of the binding of the antibody to BACE1 as measured, e.g., by a radioimmunoassay (RIA). In certain embodiments, an antibody that binds to BACE1 has an equilibrium dissociation constant (K_{D}) of ≤1 μM, ≤100 nM, ≤10 nM, ≤1 nM, ≤0.1 nM, ≤0.01 nM, or ≤0.001 nM (e.g. 10-8 M or less, e.g. from 10-8 M to 10-13 M, e.g., from 10-9 M to 10-13 M). In certain embodiments, an anti-BACE1 antibody binds to an epitope of BACE1 that is conserved among BACE1 from different species and isoforms. In other embodiments, an antibody is provided that binds to an exosite within BACE1 located outside the catalytic domain of BACE1. In one embodiment an antibody is provided that competes with the peptides identified in Kornacker et al., Biochem. 44:11567-11573 (2005), which is incorporated herein by reference in its entirety, (i.e., Peptides 1, 2, 3, 1-11, 1-10, 1-9, 1-8, 1-7, 1-6, 2-12, 3-12, 4-12, 5-12, 6-12, 7-12, 8-12, 9-12, 10-12, 4, 5, 6, 5-10, 5-9, scrambled, Y5A, P6A, Y7A, FBA, I9A, P10A and L11A) for binding to BACE1. Nonlimiting exemplary anti-BACE1 antibodies are described, e.g., in WO 2012/064836 and 2016/081639.

A “native sequence” protein herein refers to a protein comprising the amino acid sequence of a protein found in nature, including naturally occurring variants of the protein. The term as used herein includes the protein as isolated from a natural source thereof or as recombinantly produced.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

A “reference antibody” herein is an antibody that lacks a feature of a test antibody. In some embodiments, a reference antibody for an Fc-modified antibody is an antibody with the same variable regions and of the same isotype, but lacking the Fc modification(s). In some embodiments, a reference antibody for an Fc-modified antibody is an antibody with the same variable region(s) and isotype, but having a wild-type Fc.

An “antibody fragment” refers to a molecule other than an intact antibody that comprises a portion of an intact antibody that binds the antigen to which the intact antibody binds. Examples of antibody fragments are well known in the art (see, e.g., Nelson, MAbs (2010) 2(1): 77-83) and include but are not limited to Fab, Fab′, Fab′-SH, F(ab′)2, and Fv; diabodies; linear antibodies; single-chain antibody molecules including but not limited to single-chain variable fragments (scFv), fusions of light and/or heavy-chain antigen-binding domains with or without a linker (and optionally in tandem); and monospecific or multispecific antigen-binding molecules formed from antibody fragments (including, but not limited to multispecific antibodies constructed from multiple variable domains which lack Fcs).

The term “monoclonal antibody” as used herein refers to an antibody obtained from a population of substantially homogeneous antibodies, i.e., the individual antibodies comprising the population are identical and/or bind the same epitope, except for possible variants, e.g., containing naturally occurring mutations or that may arise during production of the monoclonal antibody, such variants generally being present in minor amounts. In contrast to polyclonal antibody preparations, which typically include different antibodies directed against different determinants (epitopes), each monoclonal antibody of a monoclonal antibody preparation is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method (see, e.g., Kohler et al., Nature, 256:495 (1975)), recombinant DNA methods (see, e.g., U.S. Pat. No. 4,816,567), phage-display methods (e.g., using the techniques described in Clackson et al., Nature, 352:624-628 (1991) and Marks et al., J. Mol. Biol., 222:581-597 (1991)), and methods utilizing transgenic animals containing all or part of the human immunoglobulin loci, such methods and other exemplary methods for making monoclonal antibodies being described herein. Specific examples of monoclonal antibodies herein include chimeric antibodies, humanized antibodies, and human antibodies, including antigen-binding fragments thereof.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)).

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

“Humanized” forms of non-human (e.g., murine) antibodies are chimeric antibodies that contain minimal sequence derived from non-human antibodies. For the most part, humanized antibodies are human antibodies (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. For example, in certain embodiments, a humanized antibody will comprise substantially all of at least one, and typically two, variable domains, in which all or substantially all of the HVRs (e.g., CDRs) correspond to those of a non-human antibody, and all or substantially all of the framework regions (FRs) correspond to those of a human antibody. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In certain embodiments, a humanized antibody will comprise substantially all of at least one, and typically two, variable domains, in which all or substantially all of the hypervariable regions correspond to those of a non-human antibody and all or substantially all of the FRs are those of a human antibody, except for FR substitution(s) as noted above. The humanized antibody optionally also will comprise at least a portion of an antibody constant region, typically that of a human antibody. A “humanized form” of an antibody, e.g., a non-human antibody, refers to an antibody that has undergone humanization. For further details, see Jones et al., Nature 321:522-525 (1986); Riechmann et al., Nature 332:323-329 (1988); and Presta, Curr. Op. Struct. Biol. 2:593-596 (1992).

A “human antibody” herein is an antibody comprising an amino acid sequence structure that corresponds with the amino acid sequence structure of an antibody produced by a human or a human cell or derived from a non-human source that utilizes human antibody repertoires or other human antibody-encoding sequences. This definition of a human antibody specifically excludes a humanized antibody comprising non-human antigen-binding residues. Such antibodies can be identified or made by a variety of techniques, including, but not limited to: production by transgenic animals (e.g., mice) that are capable, upon immunization, of producing human antibodies in the absence of endogenous immunoglobulin production (see, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807)); selection from phage display libraries expressing human antibodies or human antibody fragments (see, for example, McCafferty et al., Nature 348:552-553 (1990); Johnson et al., Current Opinion in Structural Biology 3:564-571 (1993); Clackson et al., Nature, 352:624-628 (1991); Marks et al., J. Mol. Biol. 222:581-597 (1991); Griffith et al., EMBO J. 12:725-734 (1993); U.S. Pat. Nos. 5,565,332 and 5,573,905); generation via in vitro activated B cells (see U.S. Pat. Nos. 5,567,610 and 5,229,275); and isolation from human antibody-producing hybridomas.

A “multispecific antibody” herein is an antibody having binding specificities for at least two different epitopes. Multispecific antibodies can be prepared as full-length antibodies or antibody fragments (e.g. F(ab′)2 bispecific antibodies). Engineered antibodies with two, three or more (e.g. four) functional antigen binding sites are also contemplated (see, e.g., US Appln No. US 2002/0004587 A1, Miller et al.). Multispecific antibodies can be prepared as full length antibodies or as antibody fragments.

Antibodies herein include “amino acid sequence variants” with altered antigen-binding or biological activity. Examples of such amino acid alterations include antibodies with enhanced affinity for antigen (e.g. “affinity matured” antibodies), and antibodies with altered Fc, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al.); and/or increased or diminished serum half-life (see, for example, WO00/42072, Presta, L.).

An “affinity modified variant” has one or more substituted hypervariable region or framework residues of a parent antibody (e.g. of a parent chimeric, humanized, or human antibody) that alter (increase or reduce) affinity. A convenient way for generating such substitutional variants uses phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity). In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and its target. Such contact residues and neighboring residues are candidates for substitution according to the techniques elaborated herein. Once such variants are generated, the panel of variants is subjected to screening and antibodies with altered affinity may be selected for further development.

The modified IgG Fc herein or an antibody or fusion protein comprising the modified IgG Fc may be conjugated with a “heterologous molecule” for example to increase half-life or stability or otherwise improve the antibody. For example, the modified IgG Fc herein or an antibody or fusion protein comprising the modified IgG Fc may be linked to one of a variety of non-proteinaceous polymers, e.g., polyethylene glycol (PEG), polypropylene glycol, polyoxyalkylenes, or copolymers of polyethylene glycol and polypropylene glycol. In some embodiments, the heterologous molecule is a therapeutic compound or a visualization agent (ie., a detectable label), and the IgG Fc is being used to transport such heterologous molecule across the BBB. Examples of heterologous molecules include, but are not limited to, a chemical compound, a peptide, a polymer, a lipid, a nucleic acid, and a protein.

The modified Fc herein may be a “glycosylation variant” such that any carbohydrate attached to the Fc is altered, either modified in presence/absence, or modified in type. For example, antibodies with a mature carbohydrate structure that lacks fucose attached to an Fc of the antibody are described in US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc of the antibody are referenced in WO 2003/011878, Jean-Mairet et al. and U.S. Pat. No. 6,602,684, Umana et al. Antibodies with at least one galactose residue in the oligosaccharide attached to an Fc of the antibody are reported in WO 1997/30087, Patel et al. See, also, WO 1998/58964 (Raju, S.) and WO 1999/22764 (Raju, S.) concerning antibodies with altered carbohydrate attached to the Fc thereof. See also US 2005/0123546 (Umana et al.) describing antibodies with modified glycosylation. Mutation of the consensus glycosylation sequence in the Fc (Asn-X-Ser/Thr at positions 297-299, where X cannot be proline), for example by mutating the Asn of this sequence to any other amino acid, by placing a Pro at position 298, or by modifying position 299 to any amino acid other than Ser or Thr should abrogate glycosylation at that position (see, e.g., Fares Al-Ejeh et al., Clin. Cancer Res. (2007) 13:5519s-5527s; Imperiali and Shannon, Biochemistry (1991) 30(18): 4374-4380; Katsuri, Biochem J. (1997) 323(Pt 2): 415-419; Shakin-Eshleman et al., J. Biol. Chem. (1996) 271: 6363-6366).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contact”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” residues are those variable domain residues other than the hypervariable region residues as herein defined. The FR of a variable domain generally consists of four FR domains: FR1, FR2, FR3 and FR4. Accordingly, the HVR and FR sequences generally appear in the following sequence in VH (or VL): FR1-H1(L1)-FR2-H2(L2)-FR3-H3(L3)-FR4. In certain embodiments, one or more FR residue may be modified to modulate the stability of the antibody or to modulate the three-dimensional positioning of one or more HVR of the antibody to, e.g., enhance binding.

A “full length antibody” is one which comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variants thereof.

The terms “full length antibody,” “intact antibody,” and “whole antibody” are used herein interchangeably to refer to an antibody having a structure substantially similar to a native antibody structure or having heavy chains that contain an Fc as defined herein.

A “naked antibody” refers to an antibody that is not conjugated to a heterologous moiety (e.g., a cytotoxic moiety or radiolabel). The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2 and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Effector functions” refer to those biological activities of an antibody that result in activation of the immune system other than activation of the complement pathway. Such activities are largely found in the Fc (such as a modified Fc herein) of an antibody. Examples of effector functions include, for example, Fc receptor binding and antibody-dependent cell-mediated cytotoxicity (ADCC). In one embodiment, the modified Fc herein essentially lacks effector function. In another embodiment, the modified Fc herein retains minimal effector function. Methods of modifying or eliminating effector function are well-known in the art and include, but are not limited to, modifying the Fc at one or more amino acid positions to eliminate effector function (Fc binding-impacting: positions 238, 239, 248, 249, 252, 254, 256, 265, 268, 269, 270, 272, 278, 289, 292, 293, 294, 295, 296, 297, 298, 301, 303, 311, 322, 324, 327, 329, 333, 335, 338, 340, 373, 376, 382, 388, 389, 414, 416, 419, 434, 435, 436, 437, 438, and 439; and modifying the glycosylation of the Fc (including, but not limited to, producing the antibody in an environment that does not permit wild-type mammalian glycosylation, removing one or more carbohydrate groups from an already-glycosylated antibody, and modifying the Fc at one or more amino acid positions to eliminate the ability of the antibody to be glycosylated at those positions (including, but not limited to N297G and N297A and D265A).

“Complement activation” functions, or properties of an antibody that enable or trigger “activation of the complement pathway” are used interchangeably, and refer to those biological activities of an antibody that engage or stimulate the complement pathway of the immune system in a subject. Such activities include, e.g., C1q binding and complement dependent cytotoxicity (CDC), and may be mediated by both the Fc portion and the non-Fc portion of the antibody. Methods of modifying or eliminating complement activation function are well-known in the art and include, but are not limited to, modifying the Fc at one or more amino acid positions to eliminate or lessen interactions with complement components or the ability to activate complement components, such as positions 270, 322, 329 and 321, known to be involved in C1q binding), and modifying or eliminating a portion of the non-Fc responsible for complement activation (i.e., eliminating or modifying the CH1 region at position 132 (see, e.g., Vidarte et al., (2001) J. Biol. Chem. 276(41): 38217-38223)).

Depending on the amino acid sequence, Fc domains, and antibodies of which they are part, can be assigned to different “classes”. There are five major classes of Fc domains: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known in the art. A heavy chain constant region comprises a CH1 domain, hinge, CH2 domain, and CH3 domain.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody.

The term “recombinant protein”, as used herein, refers to a protein (such as an Fc conjugate comprising a modified Fc herein) that is expressed by a recombinant host cell comprising nucleic acid encoding the protein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cells and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. Examples of “host cells” for producing recombinant antibodies and proteins include: (1) mammalian cells, for example, Chinese Hamster Ovary (CHO), COS, myeloma cells (including Y0 and NS0 cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia coli cells or Bacillus subtilis cells, etc.

As used herein, “specifically binding” or “binds specifically to” refers to an antibody selectively or preferentially binding to an antigen. The binding affinity is generally determined using a standard assay, such as Scatchard analysis, or surface plasmon resonance technique (e.g. using BIACORE®).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed herein.

An “effective amount” of an agent, e.g., a pharmaceutical formulation, refers to an amount effective, at dosages and for periods of time necessary, to achieve the desired therapeutic or prophylactic result.

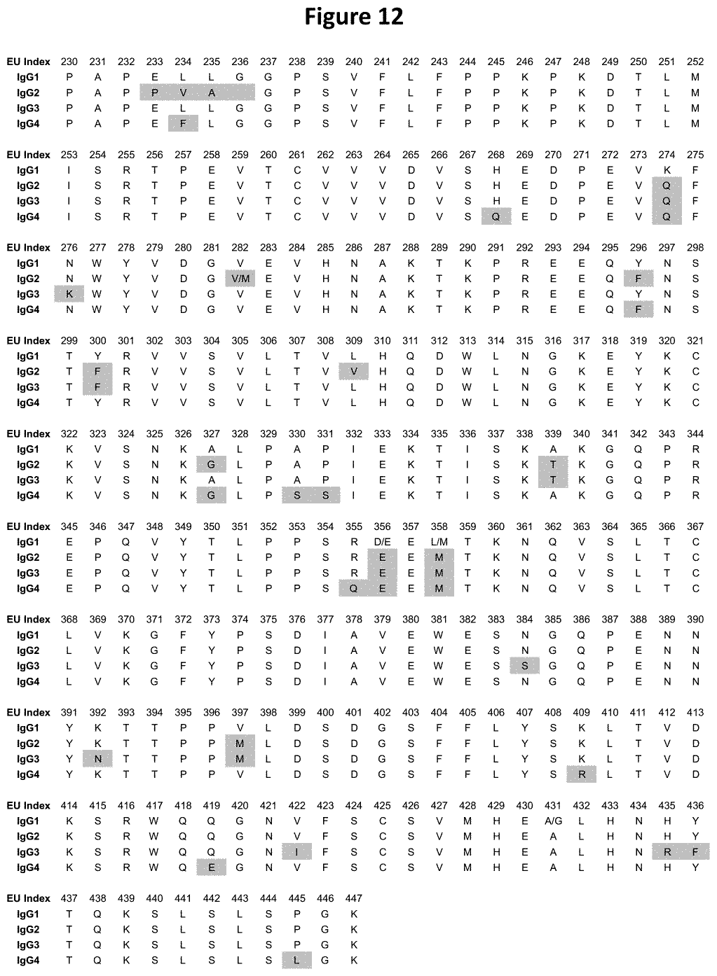

The term “Fc region” or “Fc” herein is used to define a C-terminal region of an immunoglobulin heavy chain that comprises heavy chain constant domains CH2 and CH3, or a portion of heavy chain constant domains CH2 and CH3 sufficient to bind to FcRn at pH6, pH7.4, or both pH6 and pH7.4. The term includes native sequence Fcs and modified Fcs. In some embodiments, a human IgG heavy chain Fc extends from Cys226, or from Pro230, to the carboxyl-terminus of the heavy chain. However, the C-terminal lysine (Lys447) of the Fc may or may not be present. Unless otherwise specified herein, numbering of amino acid residues in the Fc or constant region is according to the EU numbering system, also called the EU index, as described in Kabat et al., Sequences of Proteins of Immunological Interest, 5th Ed. Public Health Service, National Institutes of Health, Bethesda, Md., 1991. Exemplary human IgG1, IgG2, IgG3, and IgG4 Fc amino acid sequences are shown in FIG. 12, and SEQ ID NOs: 1-4. In some embodiments, an antibody comprising an Fc also comprises a heavy chain constant domain CH1 and the hinge.

The term “FcRn receptor” or “FcRn” as used herein refers to an Fc receptor (“n” indicates neonatal) that is known to be involved in transfer of maternal IgGs to a fetus through the human or primate placenta, or yolk sac (rabbits) and to a neonate from the colostrum through the small intestine. It is also known that FcRn is involved in the maintenance of constant serum IgG levels by binding the IgG molecules and recycling them into the serum.

A “conjugate” is an antibody or Fc conjugated to one or more heterologous molecules. An “immunoconjugate” is an antibody conjugated to one or more heterologous molecules. An “Fc conjugate” is an Fc conjugated to one or more heterologous molecules. Nonlimiting examples of such heterologous molecules include proteins, enzymes, labels, and cytotoxic agents. Optionally such conjugation is via a linker. In some embodiments, an Fc conjugate is an “Fc fusion,” in which the Fc is fused to a heterologous protein as a continuous amino acid sequence.

A “linker” as used herein is a structure that covalently or non-covalently connects a first molecule to a second molecule. In certain embodiments, a linker is a peptide. In other embodiments, a linker is a chemical linker.

A “label” is a marker coupled with the antibody herein and used for detection or imaging. Examples of such labels include: radiolabel, a fluorophore, a chromophore, or an affinity tag. In one embodiment, the label is a radiolabel used for medical imaging, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese, iron, etc.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject., A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### 1. Compositions and Methods

A. Antibodies and Fc Conjugates

In one aspect, the invention is based, in part, on modified Fcs that can be used to transport desired molecules across the BBB. In certain embodiments, antibodies comprising the modified Fcs are provided. In certain embodiments, Fc conjugates comprising the modified Fcs are provided. In some embodiments, an Fc conjugate comprises a modified Fc provided herein fused to a protein, such as a therapeutic protein and/or detectable protein. In such embodiments, the Fc conjugate may be referred to as an “Fc fusion.” Antibodies and Fc conjugates of the invention are useful, e.g., for the diagnosis or treatment of diseases affecting the brain and/or CNS.

A. Exemplary Modified Fcs

Antibodies and Fc conjugates comprising modified Fcs are provided herein, wherein the antibodies and Fc conjugates are active in an in vitro transcytosis assay. In some embodiments, the antibodies and Fc conjugates comprising modified Fcs have improved brain uptake. In some embodiments, the modified Fcs may be used to improve delivery of an antibody or Fc conjugate to the brain or central nervous system of a subject. In some embodiments, the modified Fcs herein improve transport across the blood-brain barrier (BBB).

In some embodiments, certain antibodies and Fc conjugates comprising the modified Fcs provided herein exhibit transcytosis activity in an in vitro transcytosis assay of at least 50. In some embodiments, certain antibodies and Fc conjugates comprising the modified Fcs provided herein exhibit transcytosis activity in an in vitro transcytosis assay of at least 30 or at least 40 or at least 50 when normalized to the same antibody or Fc conjugate comprising a wild-type IgG Fc. In some embodiments, certain antibodies and Fc conjugates comprising the modified Fcs provided herein exhibit a transcytosis activity in the in vitro transcytosis assay of at least 60, at least 70, at least 80, at least 90, or at least 100. A nonlimiting exemplary transcytosis assay is described in the Assays section herein. In some embodiments, the in vitro transcytosis assay comprises cells that express FcRn. In some embodiments, the FcRn is human FcRn. In some embodiments, the cells are MDCK II cells.

In some embodiments, certain antibodies and Fc conjugates comprising the modified Fcs provided herein have a binding affinity for FcRn (e.g., human FcRn) at pH 7.4 that is greater than the binding affinity of a reference antibody or Fc conjugate with an unmodified IgG Fc of the same species and isotype. In some embodiments, certain antibodies and Fc conjugates comprising the modified Fcs provided herein have a binding affinity for FcRn (e.g., human FcRn) at pH 6 that is greater than the binding affinity of a reference antibody or Fc conjugate with an unmodified IgG Fc of the same species and isotype. In some embodiments, certain antibodies and Fc conjugates comprising the modified Fcs provided herein have a binding affinity for FcRn (e.g., human FcRn) at pH 7.4 of ≤10 μM, ≤5 μM, ≤4 μM, ≤3 μM, ≤2 μM, ≤1 μM, ≤900 nM, ≤800 nM, ≤700 nM, ≤600 nM, ≤500 nM, ≤400 nM, ≤300 nM, ≤200 nM, or ≤100 nM. In some embodiments, certain antibodies and Fc conjugates comprising the modified Fcs provided herein have a binding affinity for FcRn (e.g., human FcRn) at pH 6 of ≤1 μM, ≤900 nM, ≤800 nM, ≤700 nM, ≤600 nM, ≤500 nM, ≤400 nM, ≤300 nM, ≤200 nM, ≤100 nM, ≤90 nM, ≤80 nM, ≤70 nM, ≤60 nM, ≤50 nM, ≤40 nM, ≤30 nM, ≤20 nM, or ≤10 nM. In some embodiments, the ratio of the affinity of the antibody or Fc conjugate comprising the modified IgG Fc for FcRn (e.g., human FcRn) at pH 7.4 to the affinity of the antibody or Fc conjugate comprising the modified IgG Fc for FcRn (e.g., human FcRn) at pH 6 is at least 5, at least 10, at least 20, at least 50, or at least 100; or 5 to 200, 5 to 100, 10 to 200, 10 to 100, 20 to 100, or 20 to 200.

In various embodiments, a modified Fc provided herein comprises one or more mutations selected from 252W, 252Y, 286E, 286Q, 307Q, 308P, 310A, 311A, 311I, 428L, 433K, 434F, 434W, 434Y, and 436I by EU numbering. In some embodiments, the modified Fc comprises 252Y and 434Y. In some embodiments, the modified Fc comprises 252Y and 434Y and one or two additional mutations selected from 286E, 286Q, 307Q, 308P, 311A, 311I, 428L, 433K, and 436I. In some embodiments, the modified Fc further comprises 307Q and 311A, or further comprises 286E. In some embodiments, the modified Fc comprises a set of mutations selected from the sets of mutations in Tables 4, 5, and 6. In some embodiments, the modified Fc comprises one or more modifications of an IgG sequence selected from SEQ ID NOs: 1-4. In some embodiments, the modified Fc is an IgG1 Fc. In some embodiments, the modified Fc is an IgG4 Fc. In some embodiments, the IgG Fc is an IgG2 or IgG3 Fc.

In some embodiments, a modified Fc is provided which, in the context of an antibody or Fc conjugate, has a normalized transcytosis score of at least 30. Nonlimiting examples of such modified Fcs include modified Fcs comprising the following sets of mutations: 252W/434W; 252Y/434Y; 252Y/286E/434Y; 252Y/307Q/434Y; 252Y/308P/434Y; 252Y/311A/434Y; 252Y/311I/N434Y; 252Y/428L/434Y; 252Y/433K/434Y; 252Y/434Y/436I; 286E/311A/434Y; 286E/311I/434Y; 286E/433K/434Y; 286E/434Y/436I; 307Q/286E/434Y; 307Q/311A/434Y; 307Q/311I/434Y; 307Q/433K/434Y; 307Q/434Y/436I; 311A/428L/434Y; 311A/433K/434Y; 311I/433K/434Y; 433K/434Y/436I; 252Y/307Q/311A/434Y; 252Y/307Q/311I/434Y; 252Y/307Q/434Y/436I; 252Y/311I/434Y/436I; 252Y/311A/434Y/436I; 252Y/428L/434Y/436I; 252Y/307Q/428L/434Y; and 252Y/311I/428L/434Y. In some embodiments, a modified Fc listed above is a modified IgG1 Fc. In some embodiments, a modified Fc listed above is a modified IgG4 Fc. In some embodiments, a modified Fc listed above is a modified IgG2 or IgG3 Fc.

In some embodiments, a modified Fc is provided which, in the context of an antibody or Fc conjugate, has a normalized transcytosis score of at least 70. Nonlimiting examples of such modified Fcs include modified Fcs comprising the following sets of mutations: 252W/434W; 252Y/434Y; 252Y/286E/434Y; 252Y/307Q/434Y; 252Y/308P/434Y; 252Y/311A/434Y; 252Y/311I/N434Y; 252Y/428L/434Y; 252Y/433K/434Y; 252Y/434Y/436I; 286E/311A/434Y; 286E/311I/434Y; 286E/434Y/436I; 307Q/286E/434Y; 307Q/311A/434Y; 307Q/311I/434Y; 307Q/433K/434Y; 307Q/434Y/436I; 311A/428L/434Y; 311I/433K/434Y; 433K/434Y/436I; 252Y/307Q/311A/434Y; 252Y/307Q/311I/434Y; 252Y/307Q/434Y/436I; 252Y/311I/434Y/436I; 252Y/311A/434Y/436I; 252Y/428L/434Y/436I; 252Y/307Q/428L/434Y; and 252Y/311I/428L/434Y. In some embodiments, a modified Fc listed above is a modified IgG1 Fc. In some embodiments, a modified Fc listed above is a modified IgG4 Fc. In some embodiments, a modified Fc listed above is a modified IgG2 or IgG3 Fc.

In some embodiments, a modified Fc is provided which, in the context of an antibody or Fc conjugate, has a normalized transcytosis score of at least 100. Nonlimiting examples of such modified Fcs include modified Fcs comprising the following sets of mutations: 252Y/307Q/434Y; 252Y/311A/434Y; 252Y/311I/N434Y; 252Y/428L/434Y; 252Y/433K/434Y; 252Y/434Y/436I; 286E/311A/434Y; 307Q/311I/434Y; 307Q/434Y/436I; 311A/428L/434Y; 311I/433K/434Y; 252Y/307Q/311A/434Y; 252Y/307Q/311I/434Y; 252Y/307Q/434Y/436I; 252Y/311I/434Y/436I; 252Y/311A/434Y/436I; 252Y/428L/434Y/436I; 252Y/307Q/428L/434Y; and 252Y/311I/428L/434Y. In some embodiments, a modified Fc listed above is a modified IgG1 Fc. In some embodiments, a modified Fc listed above is a modified IgG4 Fc. In some embodiments, a modified Fc listed above is a modified IgG2 or IgG3 Fc.

Nonlimiting additional modified Fcs are provided, and may be selected using an in vitro transcytosis assay, for example, as described herein. Various exemplary modified Fcs are known in the art, and may be selected using an in vitro transcytosis assay for use in the antibodies and Fc conjugates herein. Nonlimiting examples of such modified Fcs that may be assayed for transcytosis activity include those described, e.g., in US Publication Nos. 2015/0050269 and 2013/0131319, which are incorporated herein by reference in their entireties for any purpose.

1. Modified Fc Affinity

In some embodiments, a modified Fc is provided herein that has an equilibrium dissociation constant (K_{D}) of ≤10 μM, ≤5 μM, ≤4 μM, ≤3 μM, ≤2 μM, ≤1 μM, ≤900 nM, ≤800 nM, ≤700 nM, ≤600 nM, ≤500 nM, ≤400 nM, ≤300 nM, ≤200 nM, or ≤100 nM for FcRn at pH7.4. In some embodiments, a modified Fc is provided herein that as an equilibrium dissociation constant (K_{D}) of between 100 nM and 10 μM, or between 100 nM and 5 μM, or between 100 nM and 2 μM, or between 100 nM and 1 μM for FcRn at pH7.4. In some embodiments, a modified Fc is provided herein that has an equilibrium dissociation constant (K_{D}) of ≤1 μM, ≤900 nM, ≤800 nM, ≤700 nM, ≤600 nM, ≤500 nM, ≤400 nM, ≤300 nM, ≤200 nM, ≤100 nM, ≤90 nM, ≤80 nM, ≤70 nM, ≤60 nM, ≤50 nM, ≤40 nM, ≤30 nM, ≤20 nM, or ≤10 nM for FcRn at pH6. In some embodiments, a modified Fc is provided herein that as an equilibrium dissociation constant (K_{D}) of between 10 nM and 1 μM, or between 10 nM and 750 nM, or between 10 nM and 500 nM, or between 10 nM and 200 nM, or between 10 nM and 100 nM for FcRn at pH6.

In some embodiments, a modified Fc provided herein binds to FcRn at pH7.4 and binds to FcRn at pH6, wherein the ratio of the K_{D} at pH7.4 to the K_{D} at pH6 is at least 5, at least 10, at least 20, at least 50, or at least 100. In some embodiments, a modified Fc provided herein binds to FcRn at pH7.4 and binds to FcRn at pH6, wherein the ratio of the K_{D} at pH7.4 to the K_{D} at pH6 is 5 to 200, 5 to 100, 10 to 200, 10 to 100, 20 to 100, or 20 to 200.

In various embodiments, the FcRn is human FcRn.

In some embodiments, K_{D} is measured using surface plasmon resonance. In some such embodiments, K_{D} is measured using a BIACORE®-2000 device (BIAcore, Inc., Piscataway, N.J.) at 25° C. Modified Fcs are immobilized, for example, through protein-L binding at surface density of 400-1000 RU, or by using an anti-human Fab capture chip at surface density of 10-100 RU. Neutral pH binding may be determined, for example, in HBS-P (0.01 M HEPES pH 7.4, 0.15 M NaCl, 0.005% v/v Surfactant P20). Acidic pH binding may be determined, for example, in MBS-P (0.01 M MESS pH 7.5, 0.15 M NaCl, 0.005% v/v Surfactant P20). Association rates (kon) and dissociation rates (koff) are calculated using a one-to-one Langmuir binding model (BIACORE® Evaluation Software version 4.1) by simultaneously fitting the association and dissociation sensorgrams. The equilibrium dissociation constant (Kd) is calculated as the ratio koff/kon. See, e.g., Chen et al., J. Mol. Biol. 293:865-881 (1999). Alternatively, K_{D} values may be determined from the dependence of steady-state binding levels on analyte concentrations. In some embodiments, so-called steady-state analysis is particularly suited to measurement of weak to moderate interactions.

2. Modified Fc Variants

In certain embodiments, amino acid sequence variants of the modified Fcs provided herein are contemplated. For example, it may be desirable to improve the binding affinity and/or other biological properties of the modified Fc variants. Amino acid sequence variants of an Fc may be prepared by introducing appropriate modifications into the nucleotide sequence encoding the Fc, or by peptide synthesis. Such modifications include, for example, deletions from, and/or insertions into and/or substitutions of residues within the amino acid sequences of the Fc. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, Fc variants having one or more amino acid substitutions are provided. Sites of interest for substitutional mutagenesis include the HVRs and FRs. Conservative substitutions are shown in Table 2A under the heading of “preferred substitutions.” More substantial changes are provided in Table 2A under the heading of “exemplary substitutions,” and as further described below in reference to amino acid side chain classes. Amino acid substitutions may be introduced into an Fc of interest and the products screened for a desired activity, e.g., decreased immunogenicity or decreased or improved ADCC or CDC.

TABLE 2A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

A useful method for identification of residues or regions of an Fc that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the Fc with FcRn. Further substitutions may be introduced at the amino acid locations demonstrating functional sensitivity to the initial substitutions. Alternatively, or additionally, a crystal structure of an Fc-FcRn complex to identify contact points between the Fc and FcRn. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an Fc with an N-terminal methionyl residue. Other insertional variants of the Fc molecule include the fusion to the N- or C-terminus of the Fc to an enzyme (e.g. for ADEPT) or a polypeptide that increases the serum half-life of the Fc.

b) Glycosylation Variants

In certain embodiments, a modified Fc provided herein is altered to increase or decrease the extent to which the Fc is glycosylated. Addition or deletion of glycosylation sites to an Fc may be conveniently accomplished by altering the amino acid sequence such that one or more glycosylation sites is created or removed.

Native Fcs produced by mammalian cells typically comprise a branched, biantennary oligosaccharide that is generally attached by an N-linkage to Asn297 of the CH2 domain of the Fc. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an Fc of the invention may be made in order to create Fc variants with certain improved properties.

In one embodiment, Fc variants are provided having a carbohydrate structure that lacks fucose attached (directly or indirectly) to the Fc. For example, the amount of fucose in such Fc may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc (Eu numbering of Fc residues); however, Asn297 may also be located about ±3 amino acids upstream or downstream of position 297, i.e., between positions 294 and 300, due to minor sequence variations in Fcs. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” Fc variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated Fcs include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Fc variants are further provided with bisected oligosaccharides, e.g., in which a biantennary oligosaccharide attached to the Fc is bisected by GlcNAc. Such Fc variants may have reduced fucosylation and/or improved ADCC function. Examples of such Fc variants are described, e.g., in WO 2003/011878 (Jean-Mairet et al.); U.S. Pat. No. 6,602,684 (Umana et al.); and US 2005/0123546 (Umana et al.). Fc variants with at least one galactose residue in the oligosaccharide attached to the Fc are also provided. Such Fc variants may have improved CDC function. Such Fc variants are described, e.g., in WO 1997/30087 (Patel et al.); WO 1998/58964 (Raju, S.); and WO 1999/22764 (Raju, S.).

c) Cysteine Engineered Fc Variants

In certain embodiments, it may be desirable to create cysteine engineered Fc variants, in which one or more residues of a modified Fc are substituted with cysteine residues. In particular embodiments, the substituted residues occur at accessible sites of the modified Fc. By substituting those residues with cysteine, reactive thiol groups are thereby positioned at accessible sites of the modified Fc and may be used to conjugate the modified Fc to other moieties, such as drug moieties or linker-drug moieties, to create an Fc conjugate, as described further herein. Cysteine engineered Fcs may be generated as described, e.g., in U.S. Pat. Nos. 7,521,541 and 9,000,130.

d) Modified Fc Derivatives

In certain embodiments, a modified Fc provided herein may be further modified to contain additional nonproteinaceous moieties that are known in the art and readily available. The moieties suitable for derivatization of the modified Fc include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the modified Fc may vary, and if more than one polymer are attached, they can be the same or different molecules. In general, the number and/or type of polymers used for derivatization can be determined based on considerations including, but not limited to, the particular properties or functions of the modified Fc to be improved, whether the modified Fc derivative will be used in a therapy under defined conditions, etc.

In another embodiment, conjugates of a modified Fc and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the modified Fc-nonproteinaceous moiety are killed.

B. Exemplary Antibodies

In various embodiments, antibodies comprising the modified Fcs herein are provided. In certain aspects, the modified Fc may improve transport of the antibody across the BBB. In some embodiments, an antibody comprising a modified Fc herein binds to a brain antigen. Nonlimiting examples of such brain antigens include beta-secretase 1 (BACE1), amyloid beta (Abeta), epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), interleukin 6 receptor (IL6R), interleukin 1 beta (IL1β), caspase 6, triggering receptor expressed on myeloid cells 2 (TREM2), C1q, paired immunoglobin like type 2 receptor alpha (PILRA), CD33, interleukin 6 (IL6), tumor necrosis factor alpha (TNFα), tumor necrosis factor receptor superfamily member 1A (TNFR1), tumor necrosis factor receptor superfamily member 1B (TNFR2), and apolipoprotein J (ApoJ).

In a further aspect, an antibody comprising a modified Fc herein may incorporate any of the features, singly or in combination, as described in Sections 1-7 below:

1. Antibody Affinity

In certain embodiments, an antibody provided herein has an equilibrium dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³ M) for its antigen.

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

In one aspect, the RIA is a Scatchard analysis. For example, the antibody of interest can be iodinated using the lactoperoxidase method (Bennett and Horuk, Methods in Enzymology 288 pg. 134-148 (1997)). A radiolabeled antibody is purified from free ¹²⁵I-Na by gel filtration using a NAP-5 column and its specific activity measured. Competition reaction mixtures of 50 μL containing a fixed concentration of iodinated antibody and decreasing concentrations of serially diluted unlabeled antibody are placed into 96-well plates. Cells transiently expressing antigen are cultured in growth media, consisting of Dulbecco's modified eagle's medium (DMEM) (Genentech) supplemented with 10% FBS, 2 mM L-glutamine and 1× penicillin-streptomycin at 37° C. in 5% CO₂. Cells are detached from the dishes using Sigma Cell Dissociation Solution and washed with binding buffer (DMEM with 1% bovine serum albumin, 50 mM HEPES, pH 7.2, and 0.2% sodium azide). The washed cells are added at an approximate density of 200,000 cells in 0.2 mL of binding buffer to the 96-well plates containing the 50-μL competition reaction mixtures. The final concentration of the unlabeled antibody in the competition reaction with cells is varied, starting at 1000 nM and then decreasing by 1:2 fold dilution for 10 concentrations and including a zero-added, buffer-only sample. Competition reactions with cells for each concentration of unlabeled antibody are assayed in triplicate. Competition reactions with cells are incubated for 2 hours at room temperature. After the 2-hour incubation, the competition reactions are transferred to a filter plate and washed four times with binding buffer to separate free from bound iodinated antibody. The filters are counted by gamma counter and the binding data are evaluated using the fitting algorithm of Munson and Rodbard (1980) to determine the binding affinity of the antibody.

In some embodiments, K_{D} is measured using surface plasmon resonance assays with a BIACORE®-2000 device (BIAcore, Inc., Piscataway, N.J.) at 25° C. using anti-human Fc kit (BiAcore Inc., Piscataway, N.J.). Briefly, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Anti-human Fc antibody is diluted with 10 mM sodium acetate, pH 4.0, to 50 μg/ml before injection at a flow rate of 5 μl/minute to achieve approximately 10000 response units (RU) of coupled protein. Following the injection of antibody, 1 M ethanolamine is injected to block unreacted groups. For kinetics measurements, antibody is injected in HBS-P to reach about 220 RU, then two-fold serial dilutions of antigen is injected in HBS-P at 25° C. at a flow rate of approximately 30 μl/min. Association rates (kon) and dissociation rates (koff) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio koff/kon. See, e.g., Chen et al., J. Mol. Biol. 293:865-881 (1999).

Several methods of determining the IC50 for a given compound are art-known; a common approach is to perform a competition binding assay, such as that described herein. In general, a high IC50 indicates that more of the antibody is required to inhibit binding of the known ligand, and thus that the antibody's affinity for that ligand is relatively low. Conversely, a low IC50 indicates that less of the antibody is required to inhibit binding of the known ligand, and thus that the antibody's affinity for that ligand is relatively high.

2. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780). In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

3. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

4. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

5. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, bispecific antibodies may bind to two different epitopes of the same antigen. In certain embodiments, bispecific antibodies may bind to two different antigens. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies” or “dual-variable domain immunoglobulins” (DVDs) are also included herein (see, e.g. US 2006/0025576A1, and Wu et al. Nature Biotechnology (2007)).

6. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 2B under the heading of “preferred substitutions.” More substantial changes are provided in Table 2B under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or decreased or improved ADCC or CDC.

TABLE 2B

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc, the carbohydrate attached thereto may be altered. Native antibodies produced by mammalian cells typically comprise a branched, biantennary oligosaccharide that is generally attached by an N-linkage to Asn297 of the CH2 domain of the Fc. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc (Eu numbering of Fc residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Variants

In various embodiments, an antibody comprises a modified Fc provided herein. Thus, in some embodiments, one or more amino acid modifications may be introduced into the Fc of an antibody, thereby generating a modified Fc. The modified Fc may comprise a human Fc sequence (e.g., a human IgG1, IgG2, IgG3 or IgG4 Fc) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Non-limiting examples of antibodies with reduced effector function include those with substitution of one or more of Fc residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. See, e.g., U.S. Pat. Nos. 6,737,056 and 8,969,526; WO 2004/056312; and Shields et al., J. Biol. Chem. 9(2): 6591-6604 (2001).

In certain embodiments, an antibody variant comprises an Fc with one or more amino acid substitutions which improve ADCC, e.g., substitutions at positions 298, 333, and/or 334 of the Fc (EU numbering of residues).

In some embodiments, alterations are made in the Fc that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

See also Duncan & Winter, Nature 322:738-40 (1988); U.S. Pat. Nos. 5,648,260; 5,624,821; and WO 94/29351 concerning other examples of Fc variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; K149 (Kabat numbering) of the light chain; A118 (EU numbering) of the heavy chain; and 5400 (EU numbering) of the heavy chain Fc. Cysteine engineered antibodies may be generated as described, e.g., in U.S. Pat. Nos. 7,521,541 and 9,000,130.

e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

C. Recombinant Methods and Compositions

Antibodies, modified Fcs, and Fc fusions may be produced using recombinant methods and compositions known in the art. See, e.g., U.S. Pat. No. 4,816,567. In one embodiment, isolated nucleic acid encoding an antibody, modified Fc, or Fc fusion described herein is provided. In the case of antibodies, such a nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In some embodiments for expressing antibodies, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In various embodiments, a host cell is eukaryotic, e.g. a Chinese Hamster Ovary (CHO) cell or lymphoid cell (e.g., Y0, NS0, Sp20 cell). In some embodiment, a method of making an antibody, modified Fc, or Fc fusion is provided, wherein the method comprises culturing a host cell comprising a nucleic acid encoding the antibody, modified Fc, or Fc fusion, as provided above, under conditions suitable for expression of the antibody, modified Fc, or Fc fusion, and optionally recovering the antibody, modified Fc, or Fc fusion from the host cell (or host cell culture medium).

For recombinant production of an antibody, modified Fc, or Fc fusion, nucleic acid encoding an antibody, modified Fc, or Fc fusion, e.g., as described above, is isolated and inserted into one or more vectors for further cloning and/or expression in a host cell. For antibodies, such nucleic acid may be readily isolated and sequenced using conventional procedures (e.g., by using oligonucleotide probes that are capable of binding specifically to genes encoding the heavy and light chains of the antibody).

Suitable host cells for cloning or expression of protein-encoding vectors include prokaryotic or eukaryotic cells described herein. For example, Fc-containing proteins may be produced in bacteria, in particular when glycosylation and Fc effector function are not needed. For expression of polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the protein may be isolated from the bacterial cell paste in a soluble fraction and can be further purified.

In addition to prokaryotes, eukaryotic microbes such as filamentous fungi or yeast are suitable cloning or expression hosts for protein-encoding vectors, including fungi and yeast strains whose glycosylation pathways have been “humanized,” resulting in the production of a protein with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003).

D. Assays

Modified Fcs provided herein may be identified, screened for, or characterized for their physical/chemical properties and/or biological activities by various assays known in the art.

1. Binding Assays and Other Assays

Various techniques are available for determining binding of an agent comprising a modified Fc provided herein (such as an antibody or Fc conjugate, including an Fc fusion) to FcRn. One such assay is an enzyme linked immunosorbent assay (ELISA) for confirming an ability to bind to human FcRn and various pHs, such as pH7.4 and pH6. Various techniques are also available for determining binding of an antibody to its antigen, also including enzyme linked immunosorbent assay (ELISA). According to this assay, plates coated with FcRn or antigen are incubated with a sample comprising the antibody or other modified Fc-containing agent and binding of the antibody or modified Fc-containing agent to the antigen of interest or FcRn is determined.

In one aspect, an antibody of the invention is tested for its antigen binding activity, e.g., by known methods such as ELISA, Western blot, etc. In another aspect, a modified Fc-containing agent is tested for binding activity to FcRn, e.g., by known methods such as ELISA, Western blot, etc.

In another aspect, competition assays may be used to identify an antibody that competes with any of the antibodies of the invention for binding to antigen. In certain embodiments, such a competing antibody binds to the same epitope (e.g., a linear or a conformational epitope) that is bound by any of the antibodies of the invention, more specifically, any of the epitopes specifically bound by antibodies in class I, class II, class III or class IV as described herein (see, e.g., Example 1 and Table 4. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized antigen is incubated in a solution comprising a first labeled antibody that binds to antigen (e.g., one or more of the antibodies disclosed herein) and a second unlabeled antibody that is being tested for its ability to compete with the first antibody for binding to antigen. The second antibody may be present in a hybridoma supernatant. As a control, immobilized antigen is incubated in a solution comprising the first labeled antibody but not the second unlabeled antibody. After incubation under conditions permissive for binding of the first antibody to antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to antigen. See Harlow and Lane (1988) Antibodies: A Laboratory Manual ch. 14 (Cold Spring Harbor Laboratory, Cold Spring Harbor, N.Y.).

2. Activity Assays

In one aspect, assays are provided for identifying antibodies and Fc conjugates having biological activity. Biological activity may include, e.g., the ability to cross the blood-brain barrier into the brain and/or CNS and the ability to transport a compound associated with a modified Fc across the BBB into the brain and/or CNS. Antibodies and Fc conjugates having such biological activity in vivo and/or in vitro are provided.

In certain embodiments, an antibody or Fc conjugate of the invention is tested for such biological activity. In some such embodiments, the antibody or Fc conjugate is tested for such biological activity in an in vitro transcytosis assay. An exemplary transcytosis assay is as follows. MDCK II cells (American Type Culture Collection, Manassas, Va.) transfected to express human FcRn (e.g., FCGRT (UniProtKB-P55899, FCGRTN_HUMAN) and β₂m (UniProtKB—P61769, B2MG_HUMAN) separated by a P2A sequence (Kim et al PLoS one 2011; 6(4):e18556)) are seeded for 3 days in a Transwell® permeable support plate, 0.4-μm pore size (Corning Inc., Corning, N.Y.). On day 3, fresh media with test antibody and a fluorescent marker dye, such as Lucifer Yellow (Molecular Probes, Eugene, Oreg.), are added to the apical compartment. Fresh media free from test antibody and Lucifer Yellow is added to the basolateral compartment. The pH of both chambers was 7.4. Plates are incubated overnight in a 37° C., 5% CO₂ humidified incubator. On day 4, media is collected from the apical and basolateral compartments, and antibody concentration in the two compartments is assayed, e.g., by ELISA. Integrity of junction formation in the cell monolayer is monitored by measuring relative fluorescence units of Lucifer Yellow in the basolateral compartment. Data may be normalized by dividing the transcytosed concentration of each antibody or Fc conjugate, by the transcytosed concentration of a reference antibody, typically comprising a wild-type Fc.

In some embodiments, an antibody or Fc conjugate provided herein exhibits a transcytosis activity in the in vitro transcytosis assay of at least 60, at least 70, at least 80, at least 90, or at least 100. In some embodiments, an antibody or Fc conjugate provided herein exhibits a transcytosis activity in the in vitro transcytosis assay of at least 60, at least 70, at least 80, at least 90, or at least 100, when normalized to the same antibody or Fc conjugate comprising a wild-type Fc.

E. Immunoconjugates and Fc Conjugates

The invention also provides conjugates comprising an antibody or modified Fc herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes. When such conjugates comprise an antibody, in some embodiments, they may be referred to as immunoconjugates.

In one embodiment, the antibody or modified Fc herein is coupled with a neurological disorder drug, a chemotherapeutic agent and/or an imaging agent in order to more efficiently transport the drug, chemotherapeutic agent and/or the imaging agent across the BBB.

Covalent conjugation can either be direct or via a linker. In certain embodiments, direct conjugation is by construction of a protein fusion (i.e., by genetic fusion of the two genes encoding the modified Fc and e.g., the neurological disorder drug and expression as a single protein). In certain embodiments, direct conjugation is by formation of a covalent bond between a reactive group on a modified Fc or antibody and a corresponding group or acceptor on the, e.g., neurological drug. In certain embodiments, direct conjugation is by modification (i.e., genetic modification) of one of the two molecules to be conjugated to include a reactive group (as nonlimiting examples, a sulfhydryl group or a carboxyl group) that forms a covalent attachment to the other molecule to be conjugated under appropriate conditions. As one nonlimiting example, a molecule (i.e., an amino acid) with a desired reactive group (i.e., a cysteine residue) may be introduced into the antibody or modified Fc and a disulfide bond formed with the e.g., neurological drug. Methods for covalent conjugation of nucleic acids to proteins are also known in the art (i.e., photocrosslinking, see, e.g., Zatsepin et al. Russ. Chem. Rev. 74: 77-95 (2005))

Non-covalent conjugation can be by any noncovalent attachment means, including hydrophobic bonds, ionic bonds, electrostatic interactions, and the like, as will be readily understood by one of ordinary skill in the art.

Conjugation may also be performed using a variety of linkers. For example, an antibody and a neurological drug or a modified Fc and a neurological drug may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody, modified Fc, or Fc conjugate. See WO94/11026. Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine. The linker may be a “cleavable linker” facilitating release of the neurological drug upon delivery to the brain. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The invention herein expressly contemplates, but is not limited to, conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065. In some embodiments, an Fc conjugate is provided, which comprises a modified Fc herein conjugated to one or more of the forgoing drugs.

In another embodiment, a conjugate comprises an antibody or Fc described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, a conjugate comprises an antibody or Fc described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

In some embodiments, an Fc conjugate comprises a modified Fc provided herein fused to a protein, such as a therapeutic protein and/or detectable protein. In such embodiments, such Fc conjugates may be referred to as Fc fusions. Nonlimiting exemplary therapeutic proteins that may be conjugated to a modified Fc provided herein include TNF-R1, CTLA-4, IL-1R1, alpha-L-iduronidase, iduronate-2-sulphatase, N-sulfatase, alpha-N-acetylglucosaminidase, N-acetyl-galactosamine-6-sulfatase, beta-galactosidase, arylsulphatase B, beta-glucuronidase, acid alpha-glucosidase, glucocerebrosidase, alpha-galactosidase A, hexosaminidase A, acid sphingomyelinase, beta-galactocerebrosidase, beta-galactosidase, arylsulfatase A, acid ceramidase, aspartoacylase, palmitoyl-protein thioesterase 1, and tripeptidyl amino peptidase 1. In some embodiments, an extracellular domain of the therapeutic protein is conjugated to a modified Fc provided herein, such as an extracellular domain of TNF-R1, CTLA-4, or IL-1R1.

F. Methods and Compositions for Diagnostics and Detection

In some embodiments, an antibody or Fc conjugate for use in a method of diagnosis or detection is provided.

Exemplary disorders that may be diagnosed using an antibody or Fc conjugate of the invention include disorders of the central nervous system (CNS), including brain. In some embodiments, the antibodies and Fc conjugates of the invention may be used, for example, to detect antigen in the CNS (such as in the brain) in order to diagnose a disease or disorder associated with the presence of, or elevated levels of, the antigen.

In certain embodiments, labeled antibodies and Fc conjugates are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

In some embodiments, the antibody or Fc conjugate lacks effector function. In some embodiments, the antibody or Fc conjugate has reduced effector function. In another embodiment, the antibody or Fc conjugate is engineered to have reduced effector function. In some aspects, the antibody or Fc conjugate has one or more Fc mutations reducing or eliminating effector function. In another aspect, the antibody or Fc conjugate has modified glycosylation due, e.g., to producing the antibody, Fc conjugate, or modified Fc in a system lacking normal human glycosylation enzymes. In another aspect, the Ig backbone is modified to one which naturally possesses limited or no effector function.

Various techniques are available for determining binding of the antibody or Fc conjugate to the target protein. One such assay is an enzyme linked immunosorbent assay (ELISA) for confirming an ability to bind to a target protein. According to this assay, plates coated with antigen are incubated with a sample comprising the antibody or Fc conjugate and binding of the antibody or Fc conjugate to the target protein of interest is determined.

Assays for evaluating uptake of systemically administered antibody or Fc conjugate and other biological activity of the antibody or Fc conjugate can be performed as disclosed in the examples or as known in the art for the CNS target protein of interest.

In one aspect, assays are provided for identifying anti-BACE1 antibodies having biological activity. Biological activity may include, e.g., inhibition of BACE1 aspartyl protease activity. Antibodies having such biological activity in vivo and/or in vitro are also provided, e.g. as evaluated by homogeneous time-resolved fluorescence HTRF assay or a microfluidic capillary electrophoretic (MCE) assay using synthetic substrate peptides, or in vivo in cell lines which express BACE1 substrates such as APP.

F. Pharmaceutical Formulations

Pharmaceutical formulations of an antibody or Fc conjugate as described herein are prepared by mixing such antibody or Fc conjugate having the desired degree of purity with one or more optional pharmaceutically acceptable carriers, excipients or stabilizers (Remington's Pharmaceutical Sciences 16th edition, Osol, A. Ed. (1980)), in the form of lyophilized formulations or aqueous solutions. Pharmaceutically acceptable carriers, excipients, or stabilizers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or Fc conjugate formulations are described in U.S. Pat. No. 6,267,958. Aqueous antibody or Fc conjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide one or more active ingredients for treating a neuropathy disorder, a neurodegenerative disease, cancer, an ocular disease disorder, a seizure disorder, a lysosomal storage disease, an amyloidosis, a viral or microbial disease, ischemia, a behavioral disorder or CNS inflammation. Examples of such medicaments are discussed herein. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in, for example, Remington's Pharmaceutical Sciences 16th edition, Osol, A. Ed. (1980). One or more active ingredients may be encapsulated in liposomes that are coupled to antibodies or Fc conjugates described herein (see e.g., U.S. Patent Application Publication No. 20020025313).

Sustained-release preparations may be prepared. Suitable examples of sustained-release preparations include semi-permeable matrices of solid hydrophobic polymers containing the antibody or Fc conjugate, which matrices are in the form of shaped articles, e.g. films, or microcapsules. Nonlimiting examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration are generally sterile. Sterility may be readily accomplished, e.g., by filtration through sterile filtration membranes.

G. Therapeutic Methods and Compositions

Any of the antibodies or Fc conjugates provided herein may be used in therapeutic methods. In one aspect, an antibody or Fc conjugate for use as a medicament is provided. For example, the invention provides a method of transporting a therapeutic compound across the blood-brain barrier comprising exposing an antibody or Fc conjugate of the invention to the BBB such that the modified Fc allows transport of the antibody or Fc conjugate across the BBB, wherein the antibody or Fc conjugate comprises the therapeutic compound. In another example, the invention provides a method of transporting a neurological disorder drug across the blood-brain barrier comprising exposing an antibody or Fc conjugate of the invention to the BBB such that the modified Fc allows transport of the antibody or Fc conjugate across the BBB, wherein the antibody or Fc conjugate comprises the neurological disorder drug. In one embodiment, the BBB is in a mammal (e.g. a human), e.g. one which has a neurological disorder, including, without limitation: Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, traumatic brain injury, etc.

In one embodiment, the neurological disorder is selected from: a neuropathy, an amyloidosis, cancer (e.g. involving the CNS or brain), an ocular disease or disorder, a viral or microbial infection, inflammation (e.g. of the CNS or brain), ischemia, neurodegenerative disease, seizure, behavioral disorder, lysosomal storage disease, etc. The antibodies and Fc conjugates of the invention are particularly suited to treatment of such neurological disorders due to their ability to transport one or more associated active ingredients/coupled therapeutic compounds across the BBB and into the CNS/brain where such disorders find their molecular, cellular, or viral/microbial basis.

Neuropathy disorders are diseases or abnormalities of the nervous system characterized by inappropriate or uncontrolled nerve signaling or lack thereof, and include, but are not limited to, chronic pain (including nociceptive pain), pain caused by an injury to body tissues, including cancer-related pain, neuropathic pain (pain caused by abnormalities in the nerves, spinal cord, or brain), and psychogenic pain (entirely or mostly related to a psychological disorder), headache, migraine, neuropathy, and symptoms and syndromes often accompanying such neuropathy disorders such as vertigo or nausea.

For a neuropathy disorder, a neurological drug may be selected that is an analgesic including, but not limited to, a narcotic/opioid analgesic (i.e., morphine, fentanyl, hydrocodone, meperidine, methadone, oxymorphone, pentazocine, propoxyphene, tramadol, codeine and oxycodone), a nonsteroidal anti-inflammatory drug (NSAID) (i.e., ibuprofen, naproxen, diclofenac, diflunisal, etodolac, fenoprofen, flurbiprofen, indomethacin, ketorolac, mefenamic acid, meloxicam, nabumetone, oxaprozin, piroxicam, sulindac, and tolmetin), a corticosteroid (i.e., cortisone, prednisone, prednisolone, dexamethasone, methylprednisolone and triamcinolone), an anti-migraine agent (i.e., sumatriptin, almotriptan, frovatriptan, sumatriptan, rizatriptan, eletriptan, zolmitriptan, dihydroergotamine, eletriptan and ergotamine), acetaminophen, a salicylate (i.e., aspirin, choline salicylate, magnesium salicylate, diflunisal, and salsalate), a anti-convulsant (i.e., carbamazepine, clonazepam, gabapentin, lamotrigine, pregabalin, tiagabine, and topiramate), an anaesthetic (i.e., isoflurane, trichloroethylene, halothane, sevoflurane, benzocaine, chloroprocaine, cocaine, cyclomethycaine, dimethocaine, propoxycaine, procaine, novocaine, proparacaine, tetracaine, articaine, bupivacaine, carticaine, cinchocaine, etidocaine, levobupivacaine, lidocaine, mepivacaine, piperocaine, prilocaine, ropivacaine, trimecaine, saxitoxin and tetrodotoxin), and a cox-2-inhibitor (i.e., celecoxib, rofecoxib, and valdecoxib). For a neuropathy disorder with vertigo involvement, a neurological drug may be selected that is an anti-vertigo agent including, but not limited to, meclizine, diphenhydramine, promethazine and diazepam. For a neuropathy disorder with nausea involvement, a neurological drug may be selected that is an anti-nausea agent including, but not limited to, promethazine, chlorpromazine, prochlorperazine, trimethobenzamide, and metoclopramide.

Amyloidoses are a group of diseases and disorders associated with extracellular proteinaceous deposits in the CNS, including, but not limited to, secondary amyloidosis, age-related amyloidosis, Alzheimer's Disease (AD), mild cognitive impairment (MCI), Lewy body dementia, Down's syndrome, hereditary cerebral hemorrhage with amyloidosis (Dutch type); the Guam Parkinson-Dementia complex, cerebral amyloid angiopathy, Huntington's disease, progressive supranuclear palsy, multiple sclerosis; Creutzfeld Jacob disease, Parkinson's disease, transmissible spongiform encephalopathy, HIV-related dementia, amyotropic lateral sclerosis (ALS), inclusion-body myositis (IBM), and ocular diseases relating to beta-amyloid deposition (i.e., macular degeneration, drusen-related optic neuropathy, and cataract).

For amyloidosis, a neurological drug may be selected that includes, but is not limited to, an antibody or other binding molecule (including, but not limited to a small molecule, a peptide, an aptamer, or other protein binder) that specifically binds to a target selected from: beta secretase, tau, presenilin, amyloid precursor protein or portions thereof, amyloid beta peptide or oligomers or fibrils thereof, death receptor 6 (DR6), receptor for advanced glycation endproducts (RAGE), parkin, and huntingtin; a cholinesterase inhibitor (i.e., galantamine, donepezil, rivastigmine and tacrine); an NMDA receptor antagonist (i.e., memantine), a monoamine depletor (i.e., tetrabenazine); an ergoloid mesylate; an anticholinergic antiparkinsonism agent (i.e., procyclidine, diphenhydramine, trihexylphenidyl, benztropine, biperiden and trihexyphenidyl); a dopaminergic antiparkinsonism agent (i.e., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory (including, but not limited to, a nonsteroidal anti-inflammatory drug (i.e., indomethicin and other compounds listed above); a hormone (i.e., estrogen, progesterone and leuprolide); a vitamin (i.e., folate and nicotinamide); a dimebolin; a homotaurine (i.e., 3-aminopropanesulfonic acid; 3APS); a serotonin receptor activity modulator (i.e., xaliproden); an, an interferon, and a glucocorticoid.

Cancers of the CNS are characterized by aberrant proliferation of one or more CNS cell (i.e., a neural cell) and include, but are not limited to, glioma, glioblastoma multiforme, meningioma, astrocytoma, acoustic neuroma, chondroma, oligodendroglioma, medulloblastomas, ganglioglioma, Schwannoma, neurofibroma, neuroblastoma, and extradural, intramedullary or intradural tumors.

For cancer, a neurological drug may be selected that is a chemotherapeutic agent. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphor-amide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition of chemotherapeutic agents are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

Another group of compounds that may be selected as neurological drugs for cancer treatment or prevention are anti-cancer immunoglobulins (including, but not limited to, trastuzumab, pertuzumab, bevacizumab, alemtuxumab, cetuximab, gemtuzumab ozogamicin, ibritumomab tiuxetan, panitumumab and rituximab). In some instances, antibodies in conjunction with a toxic label or conjugate may be used to target and kill desired cells (i.e., cancer cells), including, but not limited to, tositumomab with a ¹³¹I radiolabel, or trastuzumab emtansine.

Ocular diseases or disorders are diseases or disorders of the eye, which for the purposes herein is considered a CNS organ segregated by the BBB. Ocular diseases or disorders include, but are not limited to, disorders of sclera, cornea, iris and ciliary body (i.e., scleritis, keratitis, corneal ulcer, corneal abrasion, snow blindness, arc eye, Thygeson's superficial punctate keratopathy, corneal neovascularisation, Fuchs' dystrophy, keratoconus, keratoconjunctivitis sicca, iritis and uveitis), disorders of the lens (i.e., cataract), disorders of choroid and retina (i.e., retinal detachment, retinoschisis, hypertensive retinopathy, diabetic retinopathy, retinopathy, retinopathy of prematurity, age-related macular degeneration, macular degeneration (wet or dry), epiretinal membrane, retinitis pigmentosa and macular edema), glaucoma, floaters, disorders of optic nerve and visual pathways (i.e., Leber's hereditary optic neuropathy and optic disc drusen), disorders of ocular muscles/binocular movement accommodation/refraction (i.e., strabismus, ophthalmoparesis, progressive external opthalmoplegia, esotropia, exotropia, hypermetropia, myopia, astigmatism, anisometropia, presbyopia and ophthalmoplegia), visual disturbances and blindness (i.e., amblyopia, Lever's congenital amaurosis, scotoma, color blindness, achromatopsia, nyctalopia, blindness, river blindness and micro-opthalmia/coloboma), red eye, Argyll Robertson pupil, keratomycosis, xerophthalmia and andaniridia.

For an ocular disease or disorder, a neurological drug may be selected that is an anti-angiogenic ophthalmic agent (i.e., bevacizumab, ranibizumab and pegaptanib), an ophthalmic glaucoma agent (i.e., carbachol, epinephrine, demecarium bromide, apraclonidine, brimonidine, brinzolamide, levobunolol, timolol, betaxolol, dorzolamide, bimatoprost, carteolol, metipranolol, dipivefrin, travoprost and latanoprost), a carbonic anhydrase inhibitor (i.e., methazolamide and acetazolamide), an ophthalmic antihistamine (i.e., naphazoline, phenylephrine and tetrahydrozoline), an ocular lubricant, an ophthalmic steroid (i.e., fluorometholone, prednisolone, loteprednol, dexamethasone, difluprednate, rimexolone, fluocinolone, medrysone and triamcinolone), an ophthalmic anesthetic (i.e., lidocaine, proparacaine and tetracaine), an ophthalmic anti-infective (i.e., levofloxacin, gatifloxacin, ciprofloxacin, moxifloxacin, chloramphenicol, bacitracin/polymyxin b, sulfacetamide, tobramycin, azithromycin, besifloxacin, norfloxacin, sulfisoxazole, gentamicin, idoxuridine, erythromycin, natamycin, gramicidin, neomycin, ofloxacin, trifluridine, ganciclovir, vidarabine), an ophthalmic anti-inflammatory agent (i.e., nepafenac, ketorolac, flurbiprofen, suprofen, cyclosporine, triamcinolone, diclofenac and bromfenac), and an ophthalmic antihistamine or decongestant (i.e., ketotifen, olopatadine, epinastine, naphazoline, cromolyn, tetrahydrozoline, pemirolast, bepotastine, naphazoline, phenylephrine, nedocromil, lodoxamide, phenylephrine, emedastine and azelastine).

Viral or microbial infections of the CNS include, but are not limited to, infections by viruses (i.e., influenza, HIV, poliovirus, rubella,), bacteria (i.e., Neisseria sp., Streptococcus sp., Pseudomonas sp., Proteus sp., E. coli, S. aureus, Pneumococcus sp., Meningococcus sp., Haemophilus sp., and Mycobacterium tuberculosis) and other microorganisms such as fungi (i.e., yeast, Cryptococcus neoformans), parasites (i.e., Toxoplasma gondii) or amoebas resulting in CNS pathophysiologies including, but not limited to, meningitis, encephalitis, myelitis, vasculitis and abscess, which can be acute or chronic.

For a viral or microbial disease, a neurological drug may be selected that includes, but is not limited to, an antiviral compound (including, but not limited to, an adamantane antiviral (i.e., rimantadine and amantadine), an antiviral interferon (i.e., peginterferon alfa-2b), a chemokine receptor antagonist (i.e., maraviroc), an integrase strand transfer inhibitor (i.e., raltegravir), a neuraminidase inhibitor (i.e., oseltamivir and zanamivir), a non-nucleoside reverse transcriptase inhibitor (i.e., efavirenz, etravirine, delavirdine and nevirapine), a nucleoside reverse transcriptase inhibitors (tenofovir, abacavir, lamivudine, zidovudine, stavudine, entecavir, emtricitabine, adefovir, zalcitabine, telbivudine and didanosine), a protease inhibitor (i.e., darunavir, atazanavir, fosamprenavir, tipranavir, ritonavir, nelfinavir, amprenavir, indinavir and saquinavir), a purine nucleoside (i.e., valacyclovir, famciclovir, acyclovir, ribavirin, ganciclovir, valganciclovir and cidofovir), and a miscellaneous antiviral (i.e., enfuvirtide, foscarnet, palivizumab and fomivirsen)), an antibiotic (including, but not limited to, an aminopenicillin (i.e., amoxicillin, ampicillin, oxacillin, nafcillin, cloxacillin, dicloxacillin, flucoxacillin, temocillin, azlocillin, carbenicillin, ticarcillin, mezlocillin, piperacillin and bacampicillin), a cephalosporin (i.e., cefazolin, cephalexin, cephalothin, cefamandole, ceftriaxone, cefotaxime, cefpodoxime, ceftazidime, cefadroxil, cephradine, loracarbef, cefotetan, cefuroxime, cefprozil, cefaclor, and cefoxitin), a carbapenem/penem (i.e., imipenem, meropenem, ertapenem, faropenem and doripenem), a monobactam (i.e., aztreonam, tigemonam, norcardicin A and tabtoxinine-beta-lactam, a beta-lactamase inhibitor (i.e., clavulanic acid, tazobactam and sulbactam) in conjunction with another beta-lactam antibiotic, an aminoglycoside (i.e., amikacin, gentamicin, kanamycin, neomycin, netilmicin, streptomycin, tobramycin, and paromomycin), an ansamycin (i.e., geldanamycin and herbimycin), a carbacephem (i.e., loracarbef), a glycopeptides (i.e., teicoplanin and vancomycin), a macrolide (i.e., azithromycin, clarithromycin, dirithromycin, erythromycin, roxithromycin, troleandomycin, telithromycin and spectinomycin), a monobactam (i.e., aztreonam), a quinolone (i.e., ciprofloxacin, enoxacin, gatifloxacin, levofloxacin, lomefloxacin, moxifloxacin, norfloxacin, ofloxacin, trovafloxacin, grepafloxacin, sparfloxacin and temafloxacin), a sulfonamide (i.e., mafenide, sulfonamidochrysoidine, sulfacetamide, sulfadiazine, sulfamethizole, sulfanilamide, sulfasalazine, sulfisoxazole, trimethoprim, trimethoprim and sulfamethoxazole), a tetracycline (i.e., tetracycline, demeclocycline, doxycycline, minocycline and oxytetracycline), an antineoplastic or cytotoxic antibiotic (i.e., doxorubicin, mitoxantrone, bleomycin, daunorubicin, dactinomycin, epirubicin, idarubicin, plicamycin, mitomycin, pentostatin and valrubicin) and a miscellaneous antibacterial compound (i.e., bacitracin, colistin and polymyxin B)), an antifungal (i.e., metronidazole, nitazoxanide, tinidazole, chloroquine, iodoquinol and paromomycin), and an antiparasitic (including, but not limited to, quinine, chloroquine, amodiaquine, pyrimethamine, sulphadoxine, proguanil, mefloquine, atovaquone, primaquine, artemesinin, halofantrine, doxycycline, clindamycin, mebendazole, pyrantel pamoate, thiabendazole, diethylcarbamazine, ivermectin, rifampin, amphotericin B, melarsoprol, efornithine and albendazole).

Inflammation of the CNS includes, but is not limited to, inflammation that is caused by an injury to the CNS, which can be a physical injury (i.e., due to accident, surgery, brain trauma, spinal cord injury, concussion) and an injury due to or related to one or more other diseases or disorders of the CNS (i.e., abscess, cancer, viral or microbial infection).

For CNS inflammation, a neurological drug may be selected that addresses the inflammation itself (i.e., a nonsteroidal anti-inflammatory agent such as ibuprofen or naproxen), or one which treats the underlying cause of the inflammation (i.e., an anti-viral or anti-cancer agent).

Ischemia of the CNS, as used herein, refers to a group of disorders relating to aberrant blood flow or vascular behavior in the brain or the causes therefor, and includes, but is not limited to: focal brain ischemia, global brain ischemia, stroke (i.e., subarachnoid hemorrhage and intracerebral hemorrhage), and aneurysm.

For ischemia, a neurological drug may be selected that includes, but is not limited to, a thrombolytic (i.e., urokinase, alteplase, reteplase and tenecteplase), a platelet aggregation inhibitor (i.e., aspirin, cilostazol, clopidogrel, prasugrel and dipyridamole), a statin (i.e., lovastatin, pravastatin, fluvastatin, rosuvastatin, atorvastatin, simvastatin, cerivastatin and pitavastatin), and a compound to improve blood flow or vascular flexibility, including, e.g., blood pressure medications.

Neurodegenerative diseases are a group of diseases and disorders associated with neural cell loss of function or death in the CNS, and include, but are not limited to: adrenoleukodystrophy, Alexander's disease, Alper's disease, amyotrophic lateral sclerosis, ataxia telangiectasia, Batten disease, cockayne syndrome, corticobasal degeneration, degeneration caused by or associated with an amyloidosis, Friedreich's ataxia, frontotemporal lobar degeneration, Kennedy's disease, multiple system atrophy, multiple sclerosis, primary lateral sclerosis, progressive supranuclear palsy, spinal muscular atrophy, transverse myelitis, Refsum's disease, and spinocerebellar ataxia.

For a neurodegenerative disease, a neurological drug may be selected that is a growth hormone or neurotrophic factor; examples include but are not limited to brain-derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-alpha, TGF-beta, vascular endothelial growth factor (VEGF), interleukin-1 receptor antagonist (IL-lra), ciliary neurotrophic factor (CNTF), glial-derived neurotrophic factor (GDNF), neurturin, platelet-derived growth factor (PDGF), heregulin, neuregulin, artemin, persephin, interleukins, glial cell line derived neurotrophic factor (GFR), granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (B MPs), netrins, saposins, semaphorins, and stem cell factor (SCF).

Seizure diseases and disorders of the CNS involve inappropriate and/or abnormal electrical conduction in the CNS, and include, but are not limited to epilepsy (i.e., absence seizures, atonic seizures, benign Rolandic epilepsy, childhood absence, clonic seizures, complex partial seizures, frontal lobe epilepsy, febrile seizures, infantile spasms, juvenile myoclonic epilepsy, juvenile absence epilepsy, Lennox-Gastaut syndrome, Landau-Kleffner Syndrome, Dravet's syndrome, Otahara syndrome, West syndrome, myoclonic seizures, mitochondrial disorders, progressive myoclonic epilepsies, psychogenic seizures, reflex epilepsy, Rasmussen's Syndrome, simple partial seizures, secondarily generalized seizures, temporal lobe epilepsy, toniclonic seizures, tonic seizures, psychomotor seizures, limbic epilepsy, partial-onset seizures, generalized-onset seizures, status epilepticus, abdominal epilepsy, akinetic seizures, autonomic seizures, massive bilateral myoclonus, catamenial epilepsy, drop seizures, emotional seizures, focal seizures, gelastic seizures, Jacksonian March, Lafora Disease, motor seizures, multifocal seizures, nocturnal seizures, photosensitive seizure, pseudo seizures, sensory seizures, subtle seizures, sylvan seizures, withdrawal seizures, and visual reflex seizures).

For a seizure disorder, a neurological drug may be selected that is an anticonvulsant or antiepileptic including, but not limited to, barbiturate anticonvulsants (i.e., primidone, metharbital, mephobarbital, allobarbital, amobarbital, aprobarbital, alphenal, barbital, brallobarbital and phenobarbital), benzodiazepine anticonvulsants (i.e., diazepam, clonazepam, and lorazepam), carbamate anticonvulsants (i.e. felbamate), carbonic anhydrase inhibitor anticonvulsants (i.e., acetazolamide, topiramate and zonisamide), dibenzazepine anticonvulsants (i.e., rufinamide, carbamazepine, and oxcarbazepine), fatty acid derivative anticonvulsants (i.e., divalproex and valproic acid), gamma-aminobutyric acid analogs (i.e., pregabalin, gabapentin and vigabatrin), gamma-aminobutyric acid reuptake inhibitors (i.e., tiagabine), gamma-aminobutyric acid transaminase inhibitors (i.e., vigabatrin), hydantoin anticonvulsants (i.e. phenytoin, ethotoin, fosphenytoin and mephenytoin), miscellaneous anticonvulsants (i.e., lacosamide and magnesium sulfate), progestins (i.e., progesterone), oxazolidinedione anticonvulsants (i.e., paramethadione and trimethadione), pyrrolidine anticonvulsants (i.e., levetiracetam), succinimide anticonvulsants (i.e., ethosuximide and methsuximide), triazine anticonvulsants (i.e., lamotrigine), and urea anticonvulsants (i.e., phenacemide and pheneturide).

Behavioral disorders are disorders of the CNS characterized by aberrant behavior on the part of the afflicted subject and include, but are not limited to: sleep disorders (i.e., insomnia, parasomnias, night terrors, circadian rhythm sleep disorders, and narcolepsy), mood disorders (i.e., depression, suicidal depression, anxiety, chronic affective disorders, phobias, panic attacks, obsessive-compulsive disorder, attention deficit hyperactivity disorder (ADHD), attention deficit disorder (ADD), chronic fatigue syndrome, agoraphobia, post-traumatic stress disorder, bipolar disorder), eating disorders (i.e., anorexia or bulimia), psychoses, developmental behavioral disorders (i.e., autism, Rett's syndrome, Aspberger's syndrome), personality disorders and psychotic disorders (i.e., schizophrenia, delusional disorder, and the like).

For a behavioral disorder, a neurological drug may be selected from a behavior-modifying compound including, but not limited to, an atypical antipsychotic (i.e., risperidone, olanzapine, apripiprazole, quetiapine, paliperidone, asenapine, clozapine, iloperidone and ziprasidone), a phenothiazine antipsychotic (i.e., prochlorperazine, chlorpromazine, fluphenazine, perphenazine, trifluoperazine, thioridazine and mesoridazine), a thioxanthene (i.e., thiothixene), a miscellaneous antipsychotic (i.e., pimozide, lithium, molindone, haloperidol and loxapine), a selective serotonin reuptake inhibitor (i.e., citalopram, escitalopram, paroxetine, fluoxetine and sertraline), a serotonin-norepinephrine reuptake inhibitor (i.e., duloxetine, venlafaxine, desvenlafaxine, a tricyclic antidepressant (i.e., doxepin, clomipramine, amoxapine, nortriptyline, amitriptyline, trimipramine, imipramine, protriptyline and desipramine), a tetracyclic antidepressant (i.e., mirtazapine and maprotiline), a phenylpiperazine antidepressant (i.e., trazodone and nefazodone), a monoamine oxidase inhibitor (i.e., isocarboxazid, phenelzine, selegiline and tranylcypromine), a benzodiazepine (i.e., alprazolam, estazolam, flurazeptam, clonazepam, lorazepam and diazepam), a norepinephrine-dopamine reuptake inhibitor (i.e., bupropion), a CNS stimulant (i.e., phentermine, diethylpropion, methamphetamine, dextroamphetamine, amphetamine, methylphenidate, dexmethylphenidate, lisdexamfetamine, modafinil, pemoline, phendimetrazine, benzphetamine, phendimetrazine, armodafinil, diethylpropion, caffeine, atomoxetine, doxapram, and mazindol), an anxiolytic/sedative/hypnotic (including, but not limited to, a barbiturate (i.e., secobarbital, phenobarbital and mephobarbital), a benzodiazepine (as described above), and a miscellaneous anxiolytic/sedative/hypnotic (i.e. diphenhydramine, sodium oxybate, zaleplon, hydroxyzine, chloral hydrate, aolpidem, buspirone, doxepin, eszopiclone, ramelteon, meprobamate and ethclorvynol)), a secretin (see, e.g., Ratliff-Schaub et al. Autism 9: 256-265 (2005)), an opioid peptide (see, e.g., Cowen et al., J. Neurochem. 89:273-285 (2004)), and a neuropeptide (see, e.g., Hethwa et al. Am. J. Physiol. 289: E301-305 (2005)).

Lysosomal storage disorders are metabolic disorders which are in some cases associated with the CNS or have CNS-specific symptoms; such disorders include, but are not limited to: Tay-Sachs disease, Gaucher's disease, Fabry disease, mucopolysaccharidosis (types I, II, III, IV, V, VI and VII), glycogen storage disease, GM1-gangliosidosis, metachromatic leukodystrophy, Farber's disease, Canavan's leukodystrophy, and neuronal ceroid lipofuscinoses types 1 and 2, Niemann-Pick disease, Pompe disease, and Krabbe's disease.

For a lysosomal storage disease, a neurological drug may be selected that is itself or otherwise mimics the activity of the enzyme that is impaired in the disease. Exemplary recombinant enzymes for the treatment of lysosomal storage disorders include, but are not limited to those set forth in e.g., U.S. Patent Application publication no. 2005/0142141 (i.e., alpha-L-iduronidase, iduronate-2-sulphatase, N-sulfatase, alpha-N-acetylglucosaminidase, N-acetyl-galactosamine-6-sulfatase, beta-galactosidase, arylsulphatase B, beta-glucuronidase, acid alpha-glucosidase, glucocerebrosidase, alpha-galactosidase A, hexosaminidase A, acid sphingomyelinase, beta-galactocerebrosidase, beta-galactosidase, arylsulfatase A, acid ceramidase, aspartoacylase, palmitoyl-protein thioesterase 1, and tripeptidyl amino peptidase 1).

In one aspect, an antibody or Fc conjugate of the invention is used to detect a neurological disorder before the onset of symptoms and/or to assess the severity or duration of the disease or disorder. In one aspect, the antibody or Fc conjugate permits detection and/or imaging of the neurological disorder, including imaging by radiography, tomography, or magnetic resonance imaging (MRI).

In one aspect, an antibody or Fc conjugate of the invention for use as a medicament is provided. In further aspects, an antibody or Fc conjugate for use in treating a neurological disease or disorder (e.g., Alzheimer's disease) is provided. In certain embodiments, an antibody or Fc conjugate for use in a method of treatment as described herein is provided. In certain embodiments, the invention provides an antibody or Fc conjugate for use in a method of treating an individual having a neurological disease or disorder comprising administering to the individual an effective amount of an antibody or Fc conjugate. In one such embodiment, the method further comprises administering to the individual an effective amount of at least one additional therapeutic agent. In further embodiments, the invention provides an antibody or Fc conjugate for use in reducing or inhibiting amyloid plaque formation in a patient at risk or suffering from a neurological disease or disorder (e.g., Alzheimer's disease). An “individual” according to any of the above embodiments is optionally a human. In certain aspects, the antibody or Fc conjugate comprising a modified Fc of the invention for use in the methods of the invention improves uptake of the neurological disorder drug as compared to an antibody or Fc conjugate that comprises a wild-type Fc.

In a further aspect, the invention provides for the use of an antibody or Fc conjugate of the invention in the manufacture or preparation of a medicament. In one embodiment, the medicament is for treatment of neurological disease or disorder. In a further embodiment, the medicament is for use in a method of treating neurological disease or disorder comprising administering to an individual having neurological disease or disorder an effective amount of the medicament. In one such embodiment, the method further comprises administering to the individual an effective amount of at least one additional therapeutic agent.

In a further aspect, the invention provides a method for treating Alzheimer's disease. In one embodiment, the method comprises administering to an individual having Alzheimer's disease an effective amount of an antibody of the invention that binds BACE1 or Abeta. In one such embodiment, the method further comprises administering to the individual an effective amount of at least one additional therapeutic agent. An “individual” according to any of the above embodiments may be a human.

The antibodies and Fc conjugates of the invention can be used either alone or in combination with other agents in a therapy. For instance, an antibody or Fc conjugate of the invention may be co-administered with at least one additional therapeutic agent. In certain embodiments, an additional therapeutic agent is a therapeutic agent effective to treat the same or a different neurological disorder as the antibody or Fc conjugate is being employed to treat. Exemplary additional therapeutic agents include, but are not limited to: the various neurological drugs described above, cholinesterase inhibitors (such as donepezil, galantamine, rovastigmine, and tacrine), NMDA receptor antagonists (such as memantine), amyloid beta peptide aggregation inhibitors, antioxidants, γ-secretase modulators, nerve growth factor (NGF) mimics or NGF gene therapy, PPARγ agonists, HMS-CoA reductase inhibitors (statins), ampakines, calcium channel blockers, GABA receptor antagonists, glycogen synthase kinase inhibitors, intravenous immunoglobulin, muscarinic receptor agonists, nicrotinic receptor modulators, active or passive amyloid beta peptide immunization, phosphodiesterase inhibitors, serotonin receptor antagonists and anti-amyloid beta peptide antibodies. In certain embodiments, the at least one additional therapeutic agent is selected for its ability to mitigate one or more side effects of the neurological drug.

Various combination therapies noted above and herein encompass combined administration (where two or more therapeutic agents are included in the same or separate formulations), and separate administration, in which case, administration of the antibody or Fc conjugate of the invention can occur prior to, simultaneously, and/or following, administration of the additional therapeutic agent and/or adjuvant. In one embodiment, administration of the antibody or Fc conjugate and administration of an additional therapeutic agent occur within about one month, or within about one, two or three weeks, or within about one, two, three, four, five or six days, of each other. Antibodies and Fc conjugates of the invention can also be used in combination with other interventional therapies such as, but not limited to, radiation therapy, behavioral therapy, or other therapies known in the art and appropriate for the neurological disorder to be treated or prevented.

An antibody or Fc conjugate of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies and Fc conjugates of the invention are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody or Fc conjugate need not be, but is optionally formulated with one or more agents currently used to prevent or treat the disorder in question or to prevent, mitigate or ameliorate one or more side effects of antibody or Fc conjugate administration. The effective amount of such other agents depends on the amount of antibody or Fc conjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or Fc conjugate of the invention (when used alone or in combination with one or more other additional therapeutic agents) will depend on the type of disease to be treated, the type of antibody or Fc conjugate, the severity and course of the disease, whether the antibody or Fc conjugate is administered for preventive or therapeutic purposes, previous therapy, the patient's clinical history and response to the antibody or Fc conjugate, and the discretion of the attending physician. The antibody or Fc conjugate is suitably administered to the patient at one time or over a series of treatments. Depending on the type and severity of the disease, about 1 mg/kg to 15 mg/kg (e.g. 0.1 mg/kg-10 mg/kg) of antibody or Fc conjugate can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody or Fc conjugate would be in the range from about 0.05 mg/kg to about 40 mg/kg. Thus, one or more doses of about 0.5 mg/kg, 2.0 mg/kg, 4.0 mg/kg, 5.0 mg/kg, 7.5 mg/kg, 10 mg/kg, 15 mg/kg, 20 mg/kg, 25 mg/kg, 30 mg/kg, 35 mg/kg or 40 mg/kg (or any combination thereof) may be administered to the patient. Such doses may be administered intermittently, e.g. every week or every three weeks (e.g. such that the patient receives from about two to about twenty, or e.g. about six doses of the antibody or Fc conjugate). An initial higher loading dose, followed by one or more lower doses may be administered. However, other dosage regimens may be useful. The progress of this therapy is easily monitored by conventional techniques and assays as described herein and as known in the art.

H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody comprising a modified Fc of the invention or an Fc conjugate of the invention. The label or package insert indicates that the composition is used for treating the condition of choice. Moreover, the article of manufacture may comprise (a) a first container with a composition contained therein, wherein the composition comprises an antibody comprising a modified Fc of the invention or an Fc conjugate of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following examples are provided to illustrate certain disclosed embodiments and are not to be construed as limiting the scope of the invention in any way.

### Example 1—Methods

Plasmid construction and antibody and FcRn production—Antibodies, antibody Fcs, and the human and murine FcRn complexes were expressed using standard techniques including cloning sequences encoding antibody heavy and light chains or Fcs or, in the case of FcRn, FCGRT or Fcgrt (which encode the human and mouse FcRn alpha-chain, respectively) and beta-2 microglobulin (β2M), into mammalian expression vectors by standard molecular biology techniques as previously described (Eaton, Wood et al. 1986). FcRn is purified by expressing with His tag and purifying by immobilized metal ion chromatography (IMAC) or by purifying over an immobilized IgG column (e.g., Sigma A0919, Mouse IgG agarose or A2909 for Rabbit IgG Agarose). Antibodies were expressed as transient transfection cultures in CHO cells (Wong, Baginski et al. 2010, Biotechol. Bioeng., 106(5): 751-63) and affinity purified over a GE MabSelect SuRe column (GE Healthcare, Pittsburgh, Pa.) followed by Superdex-200 size exclusion chromatography (GE Healthcare, Pittsburgh, Pa.).

Antibody-FcRn affinity measurements—Affinity of antibodies comprising modified Fcs to human or murine FcRn was determined by surface plasmon resonance using a Biacore T200 (GE Healthcare) instrument. All experiments were conducted at 25° C. The modified Fc antibodies were immobilized by protein-L binding at a surface density of 1000 RU. Neutral pH binding was determined in HBS-P (0.01 M HEPES pH 7.4, 0.15 M NaCl, 0.005% v/v Surfactant P20). Acidic pH binding was determined in MBS-P (0.01 M MES pH 6.0, 0.15 M NaCl, 0.005% v/v Surfactant P20). Association rates (ka) and dissociation rates (kd) were calculated using a one-to-one Langmuir binding model by simultaneous fitting of the association and dissociation sensograms (BIAevaluations version 4.1). All fits have chi-square/Rmax value of less than 10%, satisfying accepted goodness-of-fit criteria. The equilibrium dissociation constant (K_{D}) was calculated as the ratio of kd/ka. Alternatively, equilibrium binding K_{D} values were determined from the dependence of steady-state binding levels on analyte concentrations. So-called steady-state analysis is suitable for measurement of weak to moderate interactions. Undetectable binding or binding that was too weak to enable accurate affinity analysis is set to >10 uM in the tables herein.

Transcytosis Assays—An in vitro assay was used to measure transcytosis activity of the modified Fc antibodies. In this two-chamber trans-well assay, a layer of epithelial cells expressing hFcRn or mFcRn are established on a membrane separating the two chambers. The tight junctions formed between cells exclude antibody diffusion, and thus antibody passage from one chamber to the other is only possible through intracellular transport. Accordingly, transport of the antibody across this layer of cells, from one chamber to the other, is used as a model of transcytosis. A similar assay is described, e.g., in Claypool et al. Journal of Biological Chemistry 2002, Aug. 2; 277(31):28038-50. MDCK II cells (American Type Culture Collection, Manassas, Va.) were grown in Dulbecco's modified minimal essential media (Invitrogen, Gaithersburg, Md.) containing 10% fetal bovine serum (Clontech, Mountain View, Calif.), 100 units/mL of penicillin, 100 μg/mL streptomycin and 0.292 mg/mL L-Glutamine (Thermo Fisher Scientific, Waltham, Mass.) in a 37° C., 5% CO₂ humidified incubator. MDCK II cells were first transfected with genes encoding hFcRn (FCGRT (UniProtKB-P55899, FCGRTN_HUMAN) and β₂m (UniProtKB—P61769, B2MG_HUMAN) separated by a P2A sequence (Kim et al PLoS one 2011; 6(4):e18556)) and transfected cell lines were then expanded from isolated single colonies selected by FACS using an anti-FCGRT antibody (ADM31, Aldevron, Fargo, N. Dak.) and a secondary anti-mouse PE-conjugated antibody (Thermo Fisher Scientific, Waltham, Mass.). All clones were maintained with constant antibiotic selection (5 μg/mL puromycin). The final clone was chosen based on its FCGRT and β₂M cell surface expression assessed by flow cytometry using FITC anti-human β₂M (BioLegend).

The transcytosis assay was implemented as follows. FcRn-expressing cells were seeded for 3 days in a Transwell® permeable support plate, 0.4-μm pore size (Corning Inc., Corning, N.Y.). On day 3, fresh media with test antibody and a fluorescent marker dye, Lucifer Yellow (Molecular Probes, Eugene, Oreg.) were added to the apical compartment. Fresh media free from test antibody and Lucifer Yellow was added to the basolateral compartment. The pH of both chambers was 7.4. Plates were incubated overnight in a 37° C., 5% CO₂ humidified incubator. On day 4, media was collected from the apical and basolateral compartments, and antibody concentration in the two compartments was assayed by ELISA, as described below. Data was normalized by dividing the concentration of test antibody in the basolateral compartment, by the concentration of the reference antibody, typically the wild type antibody, in the same compartment. Integrity of junction formation in the cell monolayer was monitored by measuring relative fluorescence units of Lucifer Yellow in the basolateral compartment. Data from wells with elevated levels of Lucifer Yellow above a control level were discarded, as this would indicate that the epithelial barrier was not intact.

Wild-type and transgenic mouse PK and PD studies—Wild-type C57B/6 mice ages 6 to 8 weeks were used for mFcRn studies. For mouse studies to model hFcRn, transgenic Tg32 mice expressing the human FcRn alpha-chain (FCGRT) transgene under control of a human promoter and harboring a knockout allele of the mouse FcRn alpha-chain (Fcgrt^{tm1Dcr})-B6.Cg-Fcgrt^{tm1Dcr} Tg(FCGRT)32Dcr/DcrJ, (JAX stock #014565) were used (Petkova S. B. 2006 Int. Immunol 18(2): 1759-69, Roopenian, D. C., 2010, Methods Mol. Biol. 602:93-104).

Dosing, sample collection for mouse studies—Mice were intravenously injected with the indicated dose of antibody as outlined below. After various time following dosing, blood was collected and either plasma or serum isolated for measurement of antibody concentration. For plasma collection, whole blood was collected in EDTA microtainer tubes (BD Diagnostics) prior to perfusion, centrifuged at 5,000×g for 15 minutes and the supernatant was isolated for measuring plasma antibody concentrations. For serum collection, whole blood was collected in serum separator microcontainer tubes (BD Diagnostics), allowed to clot for at least 30 minutes, and spun down at 5,000×g for 90 seconds. The supernatant was isolated for serum antibody measurements. At the indicated times, mice were perfused with D-PBS, and brain was collected for measurement of antibody concentration and/or Abeta. For brain antibody concentration measurements, a hemi-brain from each mouse was homogenized in 1% NP-40 (Cal-Biochem) in PBS containing Complete Mini EDTA-free protease inhibitor cocktail tablets (Roche Diagnostics). Homogenized brain samples were rotated at 4° C. for 1 hour before centrifugation at 14,000 rpm for 20 minutes. The supernatant was isolated for brain antibody measurement. For Abeta₁₋₄₀ measurements, hemi-brains were homogenized in 5M guanidine hydrochloride buffer and samples rotated for 3 hours at room temperature prior to dilution (1:10) in 0.25% casein, 5 mM EDTA (pH 8.0) in PBS containing freshly added aprotinin (20 mg/mL) and leupeptin (10 mg/ml). Diluted homogenates were centrifuged at 14,000 rpm for 20 min. and supernatants were isolated for Abeta₁₋₄₀ measurement.

Measuring antibody concentrations in mouse plasma or serum (pharmacokinetics)—Total antibody concentrations in mouse plasma or serum were measured with a generic human Fc ELISA. Nunc 384-well MaxiSorp immunoplates were coated with F(ab′)₂ fragment of donkey anti-human IgG and Fc fragment-specific polyclonal antibody (Jackson ImmunoResearch) overnight at 4° C. Plates were blocked with PBS and 0.5% bovine serum albumin (BSA) for 1 hour at 25° C. Each antibody (control IgG or modified Fc) was used as a standard to quantify respective antibody concentrations. Plates were washed with PBS and 0.05% Tween 20 using a microplate washer (Bio-Tek Instruments Inc.), and standards and samples diluted in PBS containing 0.5% BSA, 0.35 M NaCl, 0.25% CHAPS, 5 mM EDTA, 0.05% Tween 20, and 15 ppm (parts per million) Proclin were added for 2 hours at 25° C. Bound antibody was detected with HRP-conjugated F(ab′)2 goat anti-human IgG and Fc-specific polyclonal antibody (Jackson ImmunoResearch) and developed with TMB (KPL Inc.), and absorbance (A) was measured at 450 nm on a Multiskan Ascent reader (Thermo Scientific). Concentrations were determined from the standard curve with a four-parameter nonlinear regression program.

Anti-BACE1 antibody pharmacokinetics assay—Antibody concentrations in mouse serum and brain samples were measured using an ELISA. NUNC 384 well Maxisorp immunoplates (Neptune, N.J.) were coated with recombinant human BACE1 ECD as coat overnight at 4° C. Plates were then blocked with PBS containing 0.5% BSA for 1 hour at room temperature. Each antibody was used as a standard to quantify the respective antibody concentrations in serum and brain samples. After washing plates with PBS containing 0.05% Tween 20 using a microplate washer (Bio-Tek Instruments, Inc., Winooski, Vt.), standards and samples diluted in PBS containing 0.5% BSA, 0.35 M NaCl, 0.25% CHAPS, 5 mM EDTA, 0.05% Tween 20, and 15 ppm Proclin were incubated on plates for 2 hours at room temperature with mild agitation. Bound antibody was detected with HRP-conjugated F(ab′)₂ goat anti-human IgG Fc-specific polyclonal antibody (Jackson ImmunoResearch). Finally, plates were developed using the substrate 3,3′,5,5′-tetramethyl benzidine (TMB) (KPL, Inc., Gaithersburg, Md.). Absorbance was measured at a wavelength of 450 nm with a reference of 630 nm on a Multiskan Ascent reader (Thermo Scientific, Hudson, N.H.). Concentrations were determined from the standard curve using a four-parameter nonlinear regression program. The free anti-BACE1 mouse ELISA had a lower limit of detection (LLOD) of 0.06 ng/ml.

PD Assays—Abeta₁₋₄₀ concentrations in mouse brain samples were measured using an ELISA similar to methods described above for PK analysis. Briefly, rabbit polyclonal antibody specific for the C terminus of Abeta₁₋₄₀ (Millipore, Bedford, Mass.) was coated onto plates, and biotinylated anti-mouse Abeta monoclonal antibody M3.2 (Covance, Dedham, Mass.) was used for detection. The assay had lower limit of quantification values of 1.96 pg/ml in plasma and 39.1 pg/g in brain.

Radiolabel studies—A modified Chizzonite radioiodination protocol was used to label antibodies with ¹²⁵I (Chizzonite, Truitt et al. 1991). Wild-type or human FCGRT homozygous transgenic mice (Tg32) were administered [¹²⁵I]-labeled antibody (5 mCi iv). After injection, blood, brain, and other tissues were collected at designated time points. The samples were then analyzed for total radioactivity per gram of tissue. Tissue radioactivity was corrected for contribution from vascular blood concentration (n=2 per group).

### Example 2—Pharmacokinetics and Pharmacodynamics of hIgG1 Wildtype and Fc Modified Antibodies in Wild-Type and Human FCGRT Transgenic Mice

Two antibodies comprising modified Fcs previously identified as having improved binding to hFcRn at pH6, M428L/N434A (LA) and M252Y/S254T/T256E (YTE), and two antibodies comprising wild-type hIgG1 and hIgG4 were expressed and purified and the affinities for hFcRn at pH7.4 and pH6 were measured. Additionally, transcytosis of each antibody, comprising wild-type IgG or modified Fc, was determined in an in vitro transcytosis assay as described in Example 1. Table 2 shows the affinities and transcytosis results for these antibodies.

TABLE 2
hFcRn affinity and transcytosis activity of wild-type antibodies
and antibodies with improved hFcRn binding at pH 6

| | | FcRn KD pH 7.4 | FcRn KD pH 6 | | | Transcytosis |
|---|---|---|---|---|---|---|
| Fc modifications | | Steady State | Kinetic Analysis | | | Normalized |
| Name | Mutations | Analysis (nM) | ka (1/Ms) | kd (1/s) | KD (nM) | to WT |
| WT-hIgG1 | | >10 uM | 2.23E+04 | 1.64E−02 | 732 | 1.0 |
| WT-hIgG4 | | >10 uM | 2.11E+04 | 1.19E−02 | 563 | 1.0 |
| LA | M428L/N434A | >10 uM | 3.74E+04 | 3.71E−03 | 99.2 | 5.1 |
| YTE | M252Y/S254T/ | >10 uM | 4.14E+05 | 6.35E−02 | 154 | 1.9 |
| | T256E | | | | | |

As shown in Table 2, antibodies comprising the LA or YTE Fc modifications have improved FcRn binding at pH6 and no detectable difference in binding at pH7.4, and as compared to the wild type controls, showed little improvement in transcytosis. (>10 uM indicates that the affinity at pH7.4 was undetectable or too weak to be measured.) LA and YTE variants increased transcytosis by 5.1- and 1.9-fold respectively.

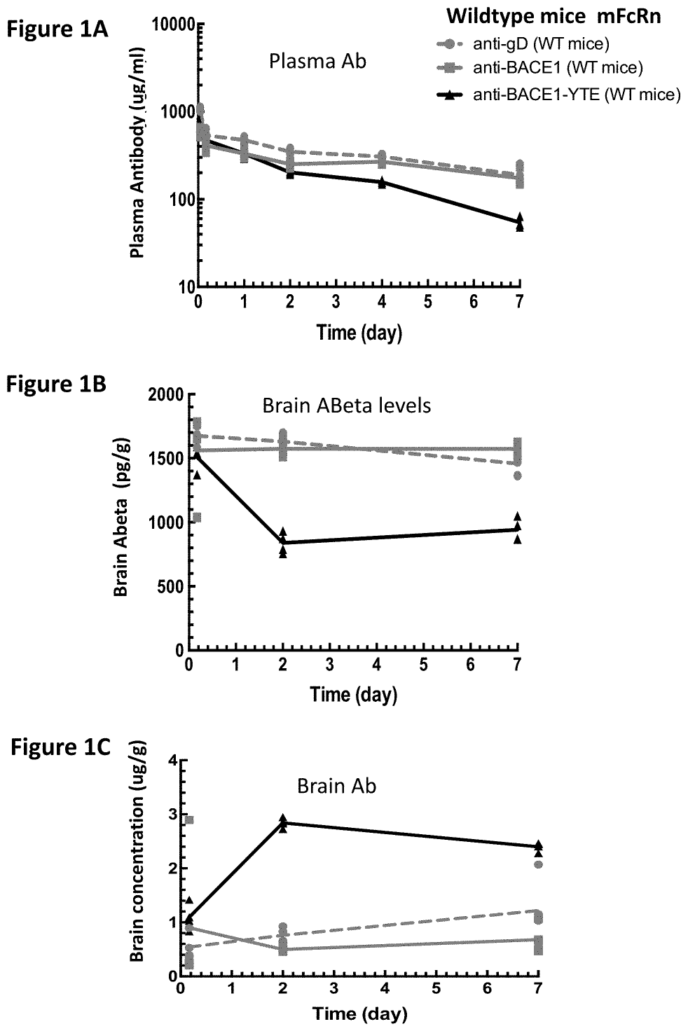

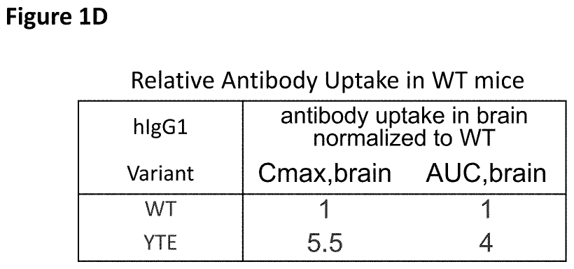

To assess whether brain uptake can be improved with antibodies enhanced for FcRn affinity at pH6, antibody pharmacokinetics and Abeta pharmacodynamics were determined in wild-type (Fcgrt^{+/+}) mice following administration of a control anti-gD hIgG1 antibody (anti-gD-hIgG1), an anti-BACE1 hIgG1 antibody (anti-BACE1-hIgG1), or an anti-BACE1 hIgG1 antibody comprising the YTE Fc modification (anti-BACE1-hIgG1-YTE). As shown in FIG. 1, anti-BACE1-hIgG1-YTE showed faster clearance from plasma (FIG. 1A), and improved brain antibody concentration (FIG. 1C), relative to an anti-BACE1 hIgG1 with wild-type Fc. Consistent with the improved brain concentration of the antibody, anti-BACE1-hIgG1-YTE administration reduced brain Abeta₁₋₄₀ levels (FIG. 1B). In contrast, anti-gD-hIgG1 (control) and anti-BACE1-hIgG1 antibodies had little effect on brain Abeta₁₋₄₀ levels, consistent with the low levels of those antibodies detected in the brain (FIGS. 1B, 1C). Relative to anti-BACE1-hIgG1, the anti-BACE1-hIgG1-YTE increased brain exposure in terms of both the maximum concentration (Cmax) and area under the curve (AUC) (FIG. 1D) in wild-type (Fcgrt^{+/+}) mice.

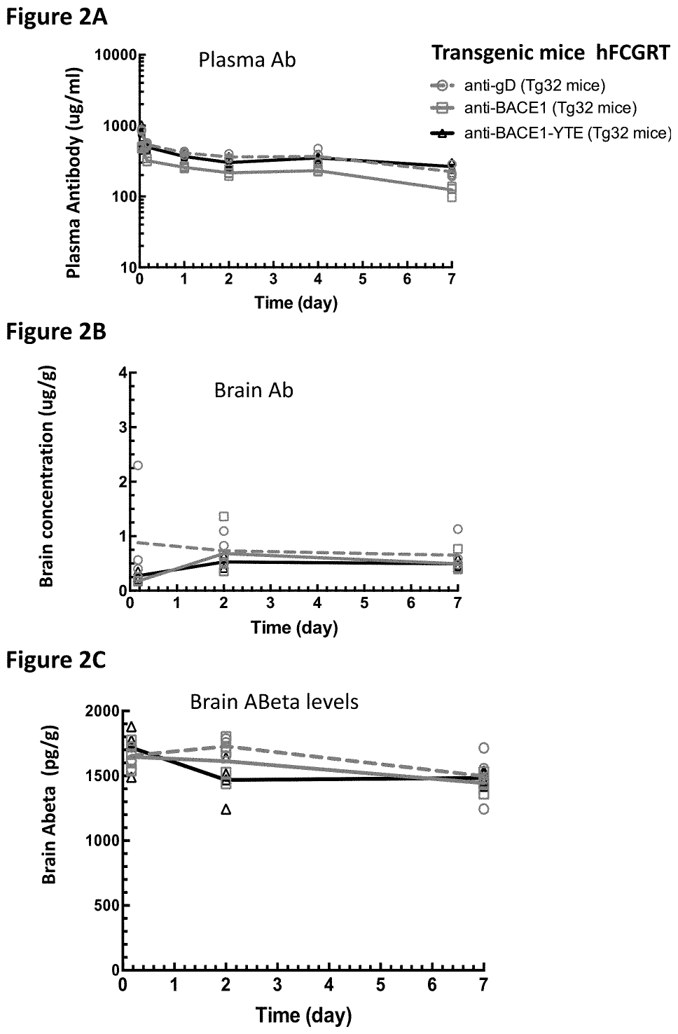

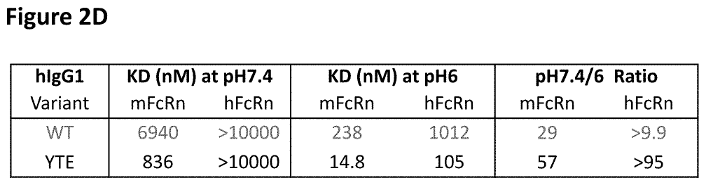

Surprisingly, when anti-BACE1-hIgG1-YTE was tested in transgenic Tg32 (FCGRT^{+/+} Fcgrt^{-/-}) mice, which express hFCGRT and lack mFCGRT, there was no improvement in brain uptake or reduction in brain Abeta₁₋₄₀ levels compared to anti-BACE1-hIgG1 (FIGS. 2A, 2B, 2C). To explore the reason for this, the binding of a wild-type hIgG1 antibody and an hIgG1 antibody comprising the YTE modification to mFcRn and hFcRn at pH7.4 and pH6 was tested. The data showed that the YTE modification improved binding to mFcRn by about 10 fold at both pH 7.4 and pH 6.0, while binding to hFcRn was improved only at pH6.0 (FIG. 2D; the K_{D} data in FIG. 2D differs slightly from the data in Table 2 because it was measured in a different experiment). These results suggested that stronger affinity for FcRn at neutral pH, but not pH6, enables increased transport into the brain.

### Example 3—Evaluation of hIgG1 and hIgG4 Fc Modifications

To identify antibodies with improved brain uptake, an anti-BACE1 antibody with a series of single Fc modifications were made. The locations of the modifications are according to EU numbering. See FIG. 12.

Antibodies comprising the Fc modifications were expressed and purified and the affinities for hFcRn at pH7.4 and pH6 were measured using Biacore, and transcytosis activities were determined as described in Example 1.

Table 3 shows Fc modified antibodies with improved binding to human FcRn at pH6.0, which did not substantially improve in vitro transcytosis.

TABLE 3
hFcRn binding affinities and transcytosis activity
of antibodies comprising single Fc modifications

| | | FcRn KD pH 7.4 | FcRn KD pH 6 | | | Transcytosis |
|---|---|---|---|---|---|---|
| Fc modifications | | Steady State | Kinetic Value | | | Normalized |
| Name | Mutations | Value (nM) | ka (1/Ms) | kd (1/s) | KD (nM) | to WT |
| S1 | M252Y | >10 uM | 1.47E+04 | 1.72E−03 | 117 | 1.7 |
| S2 | N286E | >10 uM | 1.04E+04 | 3.16E−03 | 303 | 1.7 |
| S3 | T307Q | | | | | |
| S4 | H310A | >10 uM | | | | 1.1 |
| S5 | Q311A | | | | | |
| S6 | Q311I | >10 uM | 9.19E+03 | 1.57E−03 | 171 | 1.7 |
| S7 | M428L | >10 uM | 1.13E+04 | 2.02E−03 | 178 | 2.0 |
| S8 | H433K | >10 uM | 1.32E+04 | 4.80E−03 | 363 | 2.9 |
| S9 | N434F | >10 uM | 4.18E+05 | 4.19E−02 | 100 | 6.3 |
| S10 | N434W | 5530 | 9.29E+05 | 5.10E−02 | 54.9 | 26 |
| S11 | N434Y | >10 uM | 1.91E+05 | 1.41E−02 | 73.6 | 7.8 |
| S12 | Y436I | >10 uM | 9.59E+03 | 1.93E−03 | 201 | 1.8 |

As shown in Table 3, no or modest improvements in transcytosis (1.7-7.8) was observed for modified Fc antibodies with enhanced affinity for hFcRn at pH6 but with unmeasurable affinity at pH7.4, consistent with the YTE results described in Example 2. In contrast, the weak albeit measurable pH7.4 affinity (5.5 μM) of the antibody comprising a N434W Fc modification showed an increase in transcytosis (26-fold) relative to wild-type antibody.

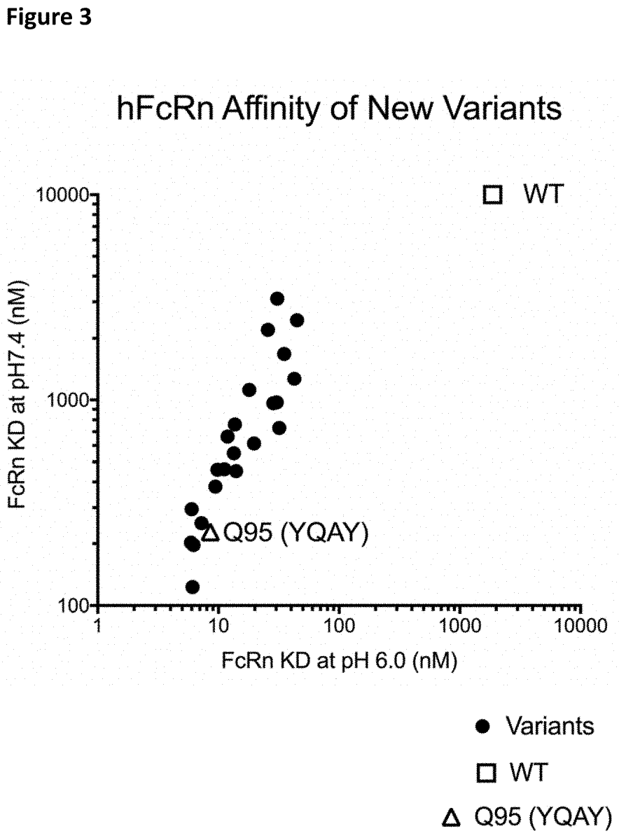

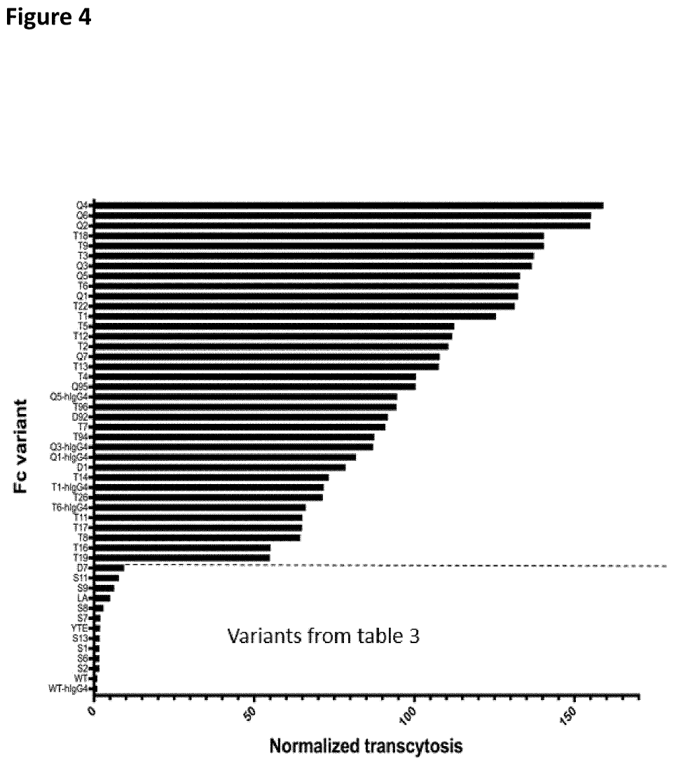

Single Fc modifications were combined into double, triple, and quadruple Fc modifications, and anti-BACE1 antibodies comprising the modified Fcs were constructed, expressed, and purified as described in Example 1, and tested for binding to human FcRn at pH7.4 and pH6, and for efficacy in the transcytosis assay described in Example 1. Tables 4, 5, and 6 show antibodies comprising double, triple, and quadruple Fc modifications, respectively, with improved binding to human FcRn at pH7.4 and improved transcytosis activity. FIG. 3 shows the correlation between pH7.4 and pH6 FcRn affinities of the modified Fc antibodies. Each dot represents a single antibody. An open triangle denotes an exemplary antibody of the present disclosure, comprising a quadruple Fc modification M252Y/T307Q/Q311A/N434Y and denoted as Q95 (YQAY) that was further tested in vivo. FIG. 4 shows the normalized transcytosis activity of the Fc modified antibodies shown in Tables 3, 4, 5, and 6.

TABLE 4
hFcRn binding affinities and transcytosis activity
of antibodies comprising double Fc modifications

| | | FcRn KD pH 7.4 | FcRn KD pH 6 | | | Transcytosis |
|---|---|---|---|---|---|---|
| Fc modifications | | Steady State | Kinetic Value | | | Normalized |
| Name | Mutations | Value (nM) | ka (1/Ms) | kd (1/s) | KD (nM) | to WT |
| D92 | M252W/N434W | 760 | 5.95E+05 | 7.40E−03 | 12.4 | 92 |
| D1 | M252Y/N434Y | 963 | 9.19E+05 | 2.64E−02 | 28.7 | 79 |
| D7 | H433K/N434Y | >10 uM | 2.08E+04 | 1.92E−03 | 92.2 | 9.4 |

TABLE 5
hFcRn binding affinities and transcytosis activity
of antibodies comprising triple Fc modifications

| | | FcRn KD pH 7.4 | FcRn KD pH 6 | | | Transcytosis |
|---|---|---|---|---|---|---|
| Fc modifications | | Steady State | Kinetic Value | | | Normalized |
| Name | Mutations | Value (nM) | ka (1/Ms) | kd (1/s) | KD (nM) | to WT |
| T94 | M252Y/N286E/ | 452 | 8.15E+05 | 1.10E−02 | 13.6 | 87 |
| | N434Y | | | | | |
| T1-IgG4 | M252Y/T307Q/ | 328 | 8.56E+05 | 8.97E−03 | 10.5 | 80 |
| | N434Y | | | | | |
| T1 | M252Y/T307Q/ | 461 | 8.74E+05 | 1.01E−02 | 12 | 125 |
| | N434Y | | | | | |
| T96 | M252Y/V308P/ | 123 | 8.87E+05 | 3.14E−03 | 3.54 | 94 |
| | N434Y | | | | | |
| T2 | M252Y/Q311A/ | 614 | 9.08E+05 | 1.78E−02 | 20 | 111 |
| | N434Y | | | | | |
| T3 | M252Y/Q311I/ | 550 | 9.02E+05 | 1.26E−02 | 14 | 137 |
| | N434Y | | | | | |
| T4 | M252Y/M428L/ | 2200 | 6.60E+05 | 2.01E−02 | 30 | 100 |
| | N434Y | | | | | |
| T5 | M252Y/H433K/ | 730 | 1.05E+06 | 3.57E−02 | 34 | 112 |
| | N434Y | | | | | |
| T6-IgG4 | M252Y/N434Y/ | 412 | 1.02E+06 | 1.01E−02 | 10.0 | 66 |
| | Y436I | | | | | |
| T6 | M252Y/N434Y/ | 459 | 8.45E+05 | 8.06E−03 | 10 | 133 |
| | Y436I | | | | | |
| T13 | N286E/Q311A/ | 2450 | 8.52E+05 | 3.27E−02 | 38 | 108 |
| | N434Y | | | | | |
| T14 | N286E/Q311I/ | 3110 | 7.72E+05 | 2.78E−02 | 36 | 73 |
| | N434Y | | | | | |
| T16 | N286E/H433K/ | 4980 | 4.06E+05 | 2.71E−02 | 66.7 | 55 |
| | N434Y | | | | | |
| T17 | N286E/N434Y/ | 6080 | 6.31E+05 | 3.34E−02 | 53 | 65 |
| | Y436I | | | | | |
| T7 | T307Q/N286E/ | 1120 | 9.31E+05 | 1.77E−02 | 19 | 91 |
| | N434Y | | | | | |
| T8 | T307Q/Q311A/ | 1680 | 8.54E+05 | 3.19E−02 | 37 | 64 |
| | N434Y | | | | | |
| T9 | T307Q/Q311I/ | 1930 | 1.48E+04 | 7.67E−03 | 520 | 140 |
| | N434Y | | | | | |
| T11 | T307Q/H433K/ | 4800 | 4.45E+05 | 1.66E−02 | 37 | 65 |
| | N434Y | | | | | |
| T12 | T307Q/N434Y/ | 4080 | 4.47E+05 | 2.44E−02 | 55 | 112 |
| | Y436I | | | | | |
| T18 | Q311A/M428L/ | | | | | 140 |
| | N434Y | | | | | |
| T19 | Q311A/H433K/ | 4480 | 1.79E+05 | 9.60E−03 | 53.5 | 55 |
| | N434Y | | | | | |
| T22 | Q311I/H433K/ | 9030 | 7.44E+05 | 4.75E−02 | 64 | 131 |
| | N434Y | | | | | |
| T26 | H433K/N434Y/ | | | | | 71 |
| | Y436I | | | | | |

TABLE 6
hFcRn binding affinities and transcytosis activity
of antibodies comprising quadruple Fc modifications

| | | FcRn KD pH 7.4 | FcRn KD pH 6 | | | Transcytosis |
|---|---|---|---|---|---|---|
| Fc modifications | | Steady State | Kinetic Value | | | Normalized |
| Name | Mutations | Value (nM) | ka (1/Ms) | kd (1/s) | KD (nM) | to WT |
| Q95 | M252Y/T307Q/ | 228 | 8.28E+05 | 6.08E−03 | 7.35 | 100 |
| | Q311A/N434Y | | | | | |
| Q1 | M252Y/T307Q/ | 197 | 7.43E+05 | 3.49E−03 | 4.69 | 132 |
| | Q311I/N434Y | | | | | |
| Q1-IgG4 | M252Y/T307Q/ | 114 | 8.13E+05 | 4.24E−03 | 5.22 | 82 |
| | Q311I/N434Y | | | | | |
| Q3 | M252Y/T307Q/ | 202 | 8.24E+05 | 3.21E−03 | 3.9 | 137 |
| | N434Y/Y436I | | | | | |
| Q3-IgG4 | M252Y/T307Q/ | 110 | 9.13E+05 | 3.53E−03 | 3.87 | 87 |
| | N434Y/Y436I | | | | | |
| Q5 | M252Y/Q311I/ | 252 | 6.16E+05 | 3.14E−03 | 5.09 | 133 |
| | N434Y/Y436I | | | | | |
| Q5-IgG4 | M252Y/Q311I/ | 322 | 1.01E+06 | 1.35E−02 | 13.4 | 95 |
| | N434Y/Y436I | | | | | |
| Q7 | M252Y/Q311A/ | 295 | 9.20E+05 | 5.29E−03 | 6 | 108 |
| | N434Y/Y436I | | | | | |
| Q6 | M252Y/M428L/ | 380 | 6.39E+05 | 4.94E−03 | 7.72 | 155 |
| | N434Y/Y436I | | | | | |
| Q2 | M252Y/T307Q/ | 663 | 5.82E+05 | 6.48E−03 | 11.1 | 155 |
| | M428L/N434Y | | | | | |
| Q4 | M252Y/Q311I/ | 1270 | 1.86E+05 | 1.07E−02 | 57.3 | 159 |
| | M428L/N434Y | | | | | |

As shown in the Tables above, a series of Fc modified antibodies were generated that had substantially improved transcytosis activity, with some Fc modifications promoting transcytosis over 100 fold relative to a wild-type IgG1 antibody.

Tables 7 and 8 show steady-state affinities of certain anti-BACE1 antibodies and certain anti-Abeta antibodies comprising modified Fcs, determined by BIACORE using an anti-human Fab capture chip.

TABLE 7
Steady state affinities of selected anti-BACE1 antibodies comprising
Fc modifications with improved binding to human FcRn

| | | pH 7.4 | pH 6 | pH 7.4 | pH 6 |
|---|---|---|---|---|---|
| Fc modifications | | hIgG1 | hIgG1 | higG4 | higG4 |
| Name | Mutations | (nM) | (nM) | (nM) | (nM) |
| D1 | M252Y/ | 1010 ± 30 | 13.0 ± 0.6 | N/A | N/A |
| | N434Y | | | | |
| T3 | M252Y/ | 307 | 9.1 | 263 | 7.4 |
| | Q311I/ | | | | |
| | N434Y | | | | |
| T94 | M252Y/ | 380 | 8.3 | 305 ± 5 | 7.035 ± 0.005 |
| | N286E/ | | | | |
| | N434Y | | | | |
| Q1 | M252Y/ | 157 ± 5 | 6.6 | 120 | 5.2 |
| | T307Q/ | | | | |
| | Q311I/ | | | | |
| | N434Y | | | | |
| Q2 | M252Y/ | 211 | 8.7 | 370 | 6.6 |
| | T307Q/ | | | | |
| | M428L/ | | | | |
| | N434Y | | | | |
| Q3 | M252Y/ | 170 | 6.5 | 124 | 4.8 |
| | T307Q/ | | | | |
| | N434Y/ | | | | |
| | Y436I | | | | |
| Q4 | M252Y/ | N/A | N/A | N/A | N/A |
| | Q311I/ | | | | |
| | M428L/ | | | | |
| | N434Y | | | | |
| Q5 | M252Y/ | 220 | 8.8 | 230 | 7.4 |
| | Q311I/ | | | | |
| | N434Y/ | | | | |
| | Y436I | | | | |
| Q6 | M252Y/ | 194 | 8.9 | 215 | 6.3 |
| | M428L/ | | | | |
| | N434Y/ | | | | |
| | Y436I | | | | |
| Q7 | M252Y/ | 269 | 7.3 | 230 | N/A |
| | Q311A/ | | | | |
| | N434Y/ | | | | |
| | Y436I | | | | |
| Q95 | M252Y/ | 240 ± 30 | 6.9 ± 0.5 | 202 ± 13 | 5.9 ± 0.4 |
| | T307Q/ | | | | |
| | Q311A/ | | | | |
| | N434Y | | | | |
| WT | — | >7040 | >704 | >7040 | >704 |

TABLE 8
Steady state affinities of selected anti-Abeta antibodies comprising
Fc modifications with improved binding to human FcRn

| | | pH 7.4 | pH 6 | pH 7.4 | pH 6 |
|---|---|---|---|---|---|
| Fc modifications | | hIgG1 | hlgG1 | higG4 | higG4 |
| Name | Mutations | (nM) | (nM) | (nM) | (nM) |
| D1 | M252Y/N434Y | 1150 | 14.0 | 1020 | 13.1 |
| T3 | M252Y/Q311I/ | 554 | 11.6 | 334 | 9.2 |
| | N434Y | | | | |
| T94 | M252Y/N286E/ | 521 | 10.1 | 406 | 8.8 |
| | N434Y | | | | |
| Q1 | M252Y/T307Q/ | 206 ± 16 | 12.7 | 165 ± 14 | 6.3 |
| | Q311I/N434Y | | | | |
| Q2 | M252Y/T307Q/ | 482 | 10.2 | 339 | 7.7 |
| | M428L/N434Y | | | | |
| Q3 | M252Y/T307Q/ | 206 | 7.7 | 158 | 5.9 |
| | N434Y/Y436I | | | | |
| Q4 | M252Y/Q311I/ | 667 | 12.8 | 486 | 9.2 |
| | M428L/N434Y | | | | |
| Q5 | M252Y/Q311I/ | 265 | 10.8 | 151 | 7.7 |
| | N434Y/Y436I | | | | |
| Q6 | M252Y/M428L/ | 367 | 10.2 | 285 | 8.0 |
| | N434Y/Y436I | | | | |
| Q7 | M252Y/Q311A/ | 292 | 8.2 | 273 | 7.4 |
| | N434Y/Y436I | | | | |
| Q95 | M252Y/T307Q/ | 340 ± 20 | 8.2 ± 0.7 | 284 ± 18 | 6.5 ± 0.3 |
| | Q311A/N434Y | | | | |
| WT | — | >7040 | >704 | >7040 | >704 |

### Example 4—Pharmacokinetics of Antibodies Comprising Modified Fcs in Tg32 Mice

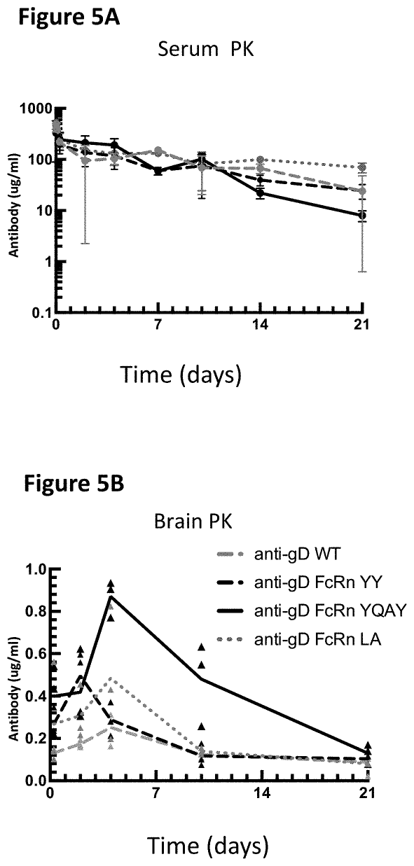

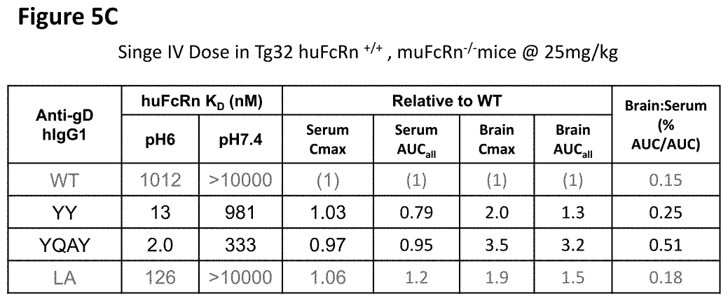

A single 25 mg/kg intravenous (IV) dose of anti-gD antibody comprising a wild-type human IgG1 (anti-gD-hIgG1), anti-gD antibody comprising a hIgG1 with a YY (D1, M252Y/N434Y) Fc modification (anti-gD-hIgG1-YY), anti-gD antibody comprising a hIgG1 with a YQAY (Q95, M252Y/T307Q/Q311A/N434Y) Fc modification (anti-gD-hIgG1-YQAY), or anti-gD antibody comprising a hIgG1 with a LA (M428L/N434A) Fc modification (anti-gD-IgG1-LA) was administered to transgenic Tg32 (FCGRT^{+/+} Fcgrt^{-/-}) mice, which express hFCGRT and lack mFCGRT. As shown in FIGS. 5A and 5B, while serum levels of anti-gD antibodies comprising the different hIgG1 Fcs were similar, brain levels of anti-gD-hIgG1-YY, anti-gD-hIgG1-YQAY, and anti-gD-hIgG1-LA were higher than anti-gD-hIgG1.

Binding affinities of the anti-gD antibodies for hFcRn were measured at pH6 and pH7.4 as described above. The YY and YQAY Fc modified antibodies provided similar hFcRn affinity enhancements in the anti-gD antibody (FIG. 5C) as was observed in the anti-BACE1 context (Tables 4 and 6). PK results and hFcRn affinities are summarized together in FIG. 5C, which shows that anti-gD-hIgG1-YY and anti-gD-hIgG1-YQAY had greater brain/serum ratios (measured as % AUC/AUC for brain/serum). Both Fc modifications resulted in significantly improved binding to hFcRn at pH7.4 and pH6 and high transcytosis scores (79 for YY and 100 for YQAY in the context of anti-BACE1—Tables 4 and 6).

### Example 5—Pharmacodynamics of Antibodies Comprising Modified Fcs in Tg32 Mice

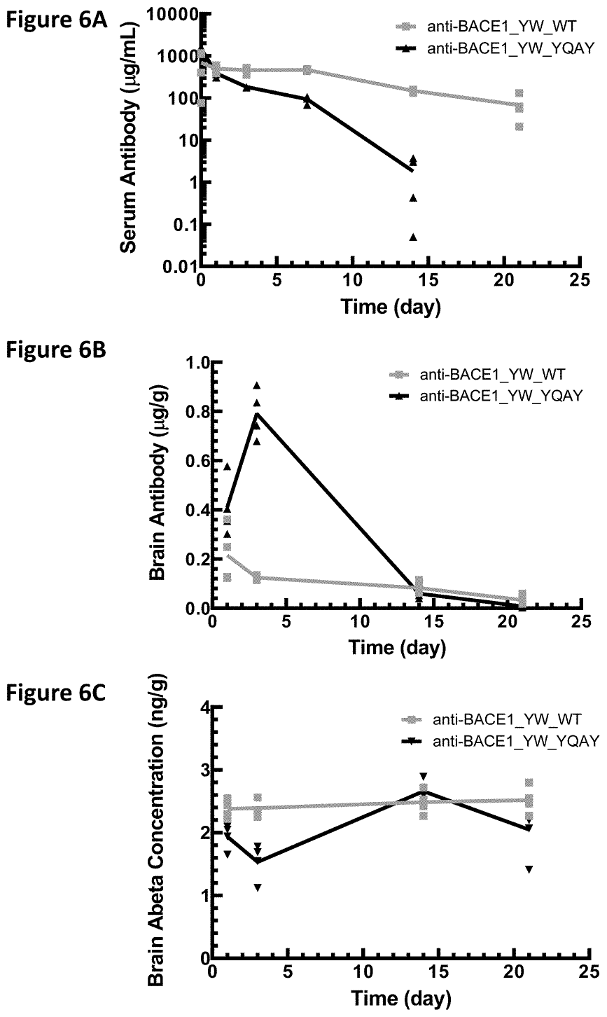

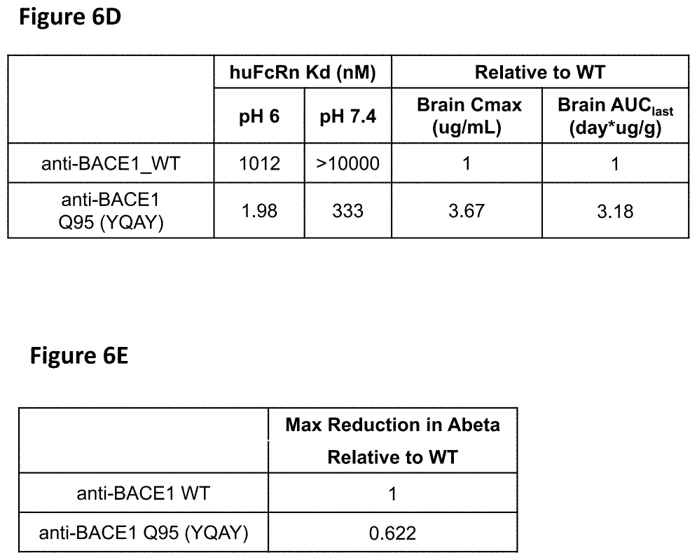

Pharmacodynamic (PD) assays were carried out to assess Abeta levels following a single 50 mg/kg IV administration of an anti-BACE1 antibody comprising wild-type hIgG1 (anti-BACE1-hIgG1) and an anti-BACE1 antibody comprising hIgG1 with a YQAY Fc modification (anti-BACE1-hIgG1-YQAY) to transgenic Tg32 (FCGRT^{+/+} Fcgrt^{-/-}) mice, which express hFCGRT and lack mFCGRT. Brain antibody levels and Abeta₁₋₄₀ levels were assayed as described in Example 1. Consistent with previous results, Fc modified anti-BACE1-hIgG1-YQAY showed greater brain uptake than anti-BACE1-hIgG1 wild-type (FIG. 6B). Further, brain Abeta₁₋₄₀ levels were reduced following administration of Fc modified anti-BACE1-hIgG1-YQAY, while anti-BACE1-hIgG1 showed little or no reduction (FIG. 6C). As shown in FIGS. 6D and 6E, the anti-BACE1-hIgG1-YQAY had a greater Cmax and AUCₗₐₛₜ than anti-BACE1-hIgG1, as well as a greater reduction in Abeta.

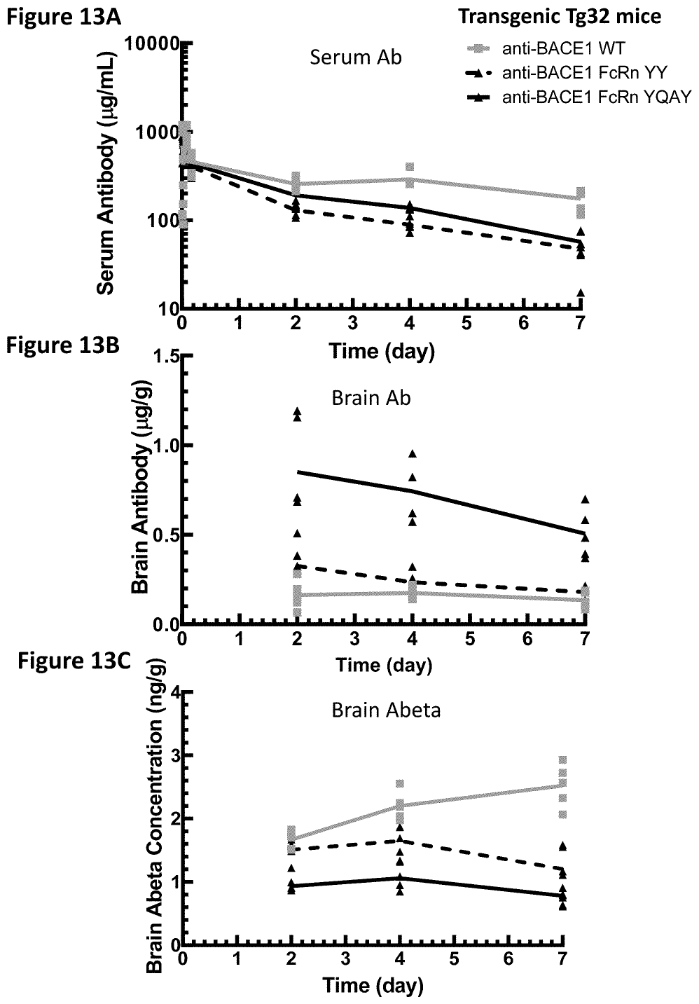

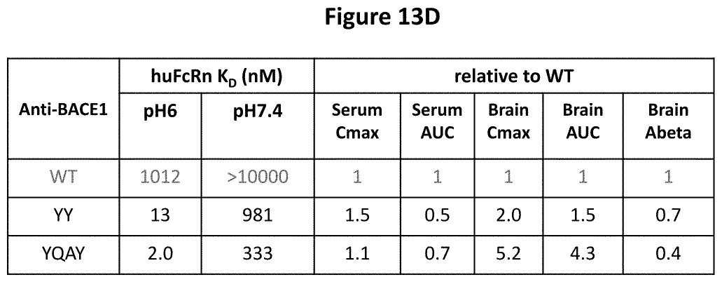

A similar experiment was conducted with a different anti-BACE1 antibody, formatted as anti-BACE1-hIgG1, anti-BACE1-hIgG1-YY, and anti-BACE1-hIgG1-YQAY. Transgenic Tg32 (FCGRT^{+/+} Fcgrt^{-/-}) mice received a single 50 mg/kg IV administration of anti-BACE1-hIgG1, anti-BACE1-hIgG1-YY, or anti-BACE1-hIgG1-YQAY. Brain antibody levels and Abeta₁₋₄₀ levels were assayed as described in Example 1. While all three antibodies had similar serum concentrations (FIG. 13A), anti-BACE1-hIgG1-YQAY showed the greatest brain uptake and anti-BACE1-hIgG1-YY trended towards enhanced brain uptake compared to anti-BACE1-hIgG1 (FIG. 13B). Consistent with the greater brain uptake, brain Abeta₁₋₄₀ levels were reduced to the greatest extent following administration of Fc modified anti-BACE1-hIgG1-YQAY, and a trend toward reduction was observed following administration of Fc modified anti-BACE1-hIgG1-YY (FIG. 13C). As shown in FIG. 13D, anti-BACE1-hIgG1-YQAY had a greater brain Cmax and AUC, and anti-BACE1-hIgG1-YY trended toward greater brain Cmax and AUC, than anti-BACE1-hIgG1, as well as a greater reduction in Abeta.

### Example 6—Pharmacokinetics of High Transcytosis IgG1 Variants in FCGRT^{+/+}, FCGRT^{+/-}, and Fcgrt^{+/+} Mice

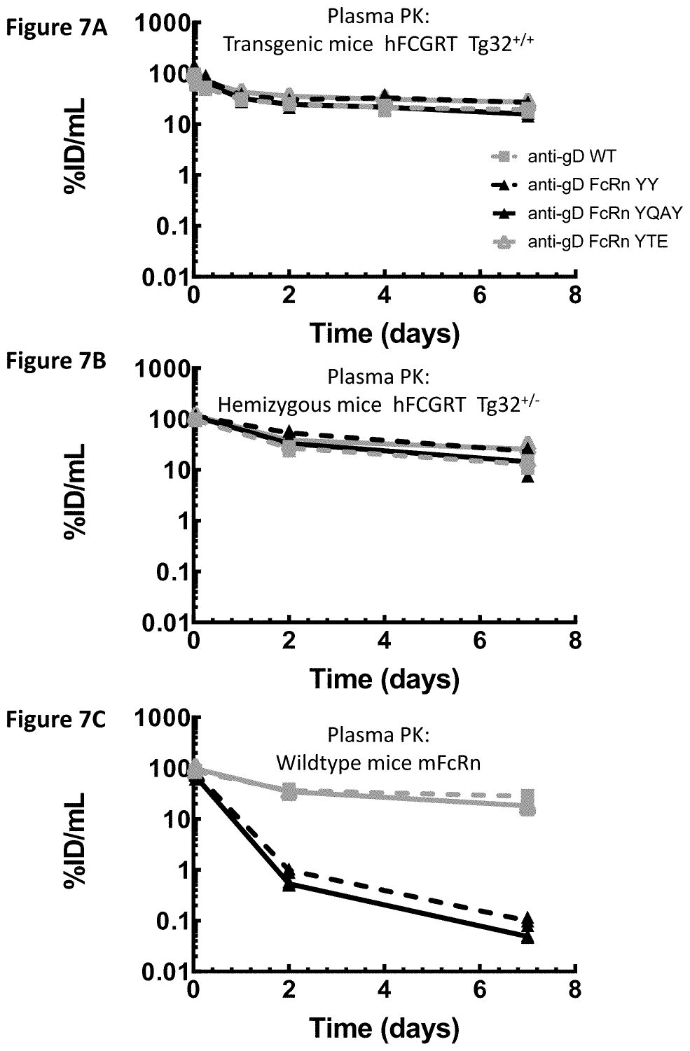

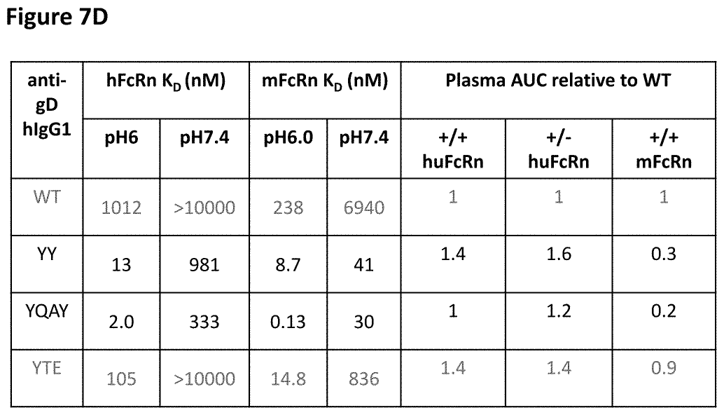

A single intravenous (IV) dose of 5 mg/kg of anti-gD-hIgG1 antibody, anti-gD-hIgG1-YY, anti-gD-hIgG1-YQAY, or anti-gD antibody comprising hIgG1 with a YTE (M252Y/S254T/T256E) Fc modification (anti-gD-hIgG1-YTE) was administered to homozygous transgenic Tg32 (FCGRT^{+/+} Fcgrt^{-/-}) mice, which express hFCGRT and lack mFCGRT; hemizygous (FCGRT^{+/-} Fcgrt^{+/-}) mice, which express both hFCGRT and mFCGRT; and wild-type (Fcgrt^{+/+}) mice, which express only mFCGRT. Each antibody was labeled with 5 μCi ¹²⁵I. All antibodies showed similar plasma PK in the homozygous Tg32 mice and hemizygous (FCGRT^{+/-} Fcgrt^{+/-}) mice. In the wild-type mice, anti-gD-hIgG1-YY and anti-gD-hIgG1-YQAY showed faster clearance from plasma than anti-gD-hIgG1 or anti-gD-hIgG1-YTE (FIGS. 7A, 7B, 7C). FIG. 7D shows affinities of anti-gD-hIgG1 and each of the Fc modified antibodies for hFcRn and mFcRn at pH7.4 and pH6, together with the plasma AUC relative to wild-type for each antibody in each strain of mice.

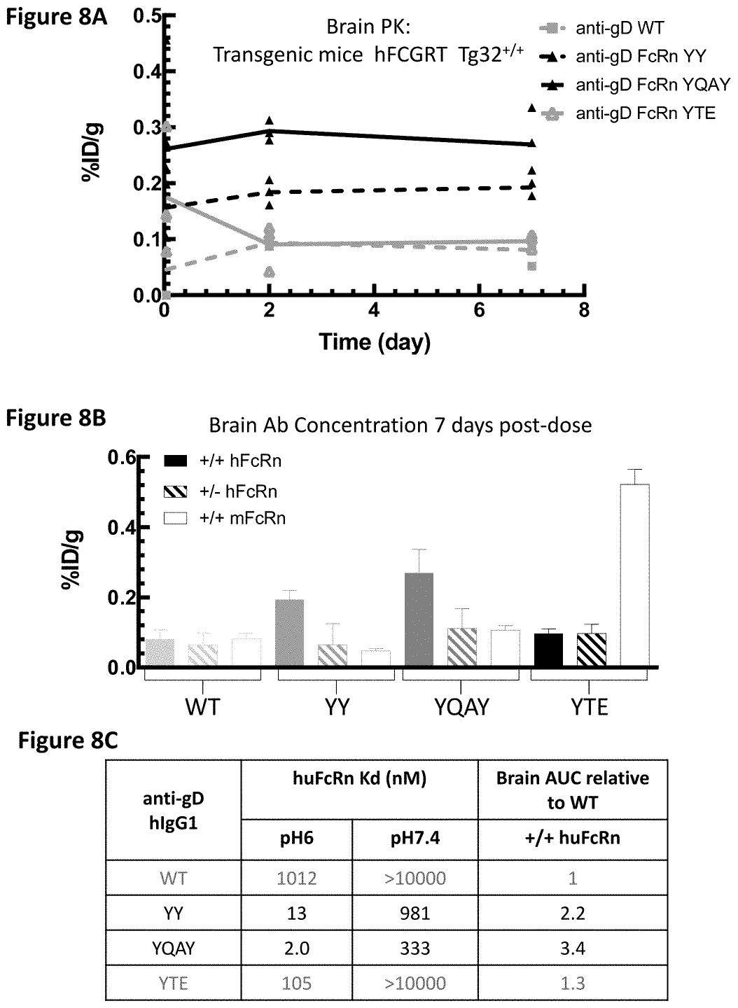

Brain PK for anti-gD-hIgG1 and the three Fc modified antibodies in homozygous hFCGRT mice is shown in FIG. 8. anti-gD-hIgG1-YY and anti-gD-hIgG1-YQAY showed greater brain uptake than anti-gD-hIgG1 or anti-gD-hIgG1-YTE (FIG. 8A). The increased brain uptake of anti-gD-hIgG1-YY and anti-gD-hIgG1-YQAY was specific to the homozygous hFCGRT Tg32 mice (FIG. 8B). As measured by AUC in mice homozygous for hFCGRT, anti-gD-hIgG1-YY provided a 2.2-fold increased brain exposure compared to anti-gD-hIgG1, and anti-gD-hIgG1-YQAY provided an even greater increase in brain exposure, a 3.4-fold increase compared to anti-gD-hIgG1 (FIG. 8C).

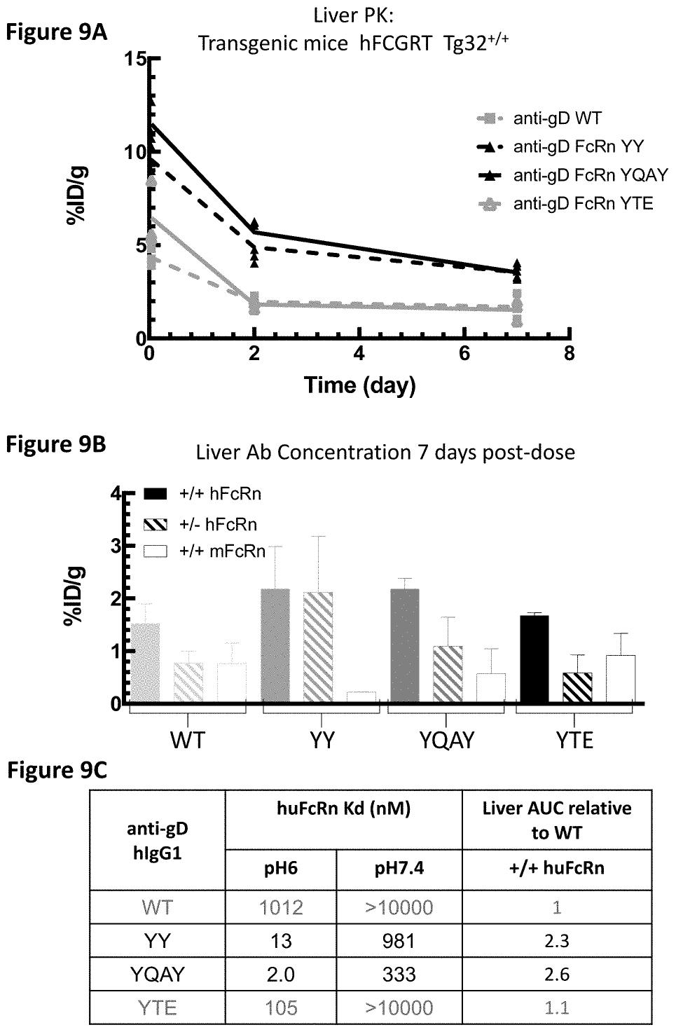

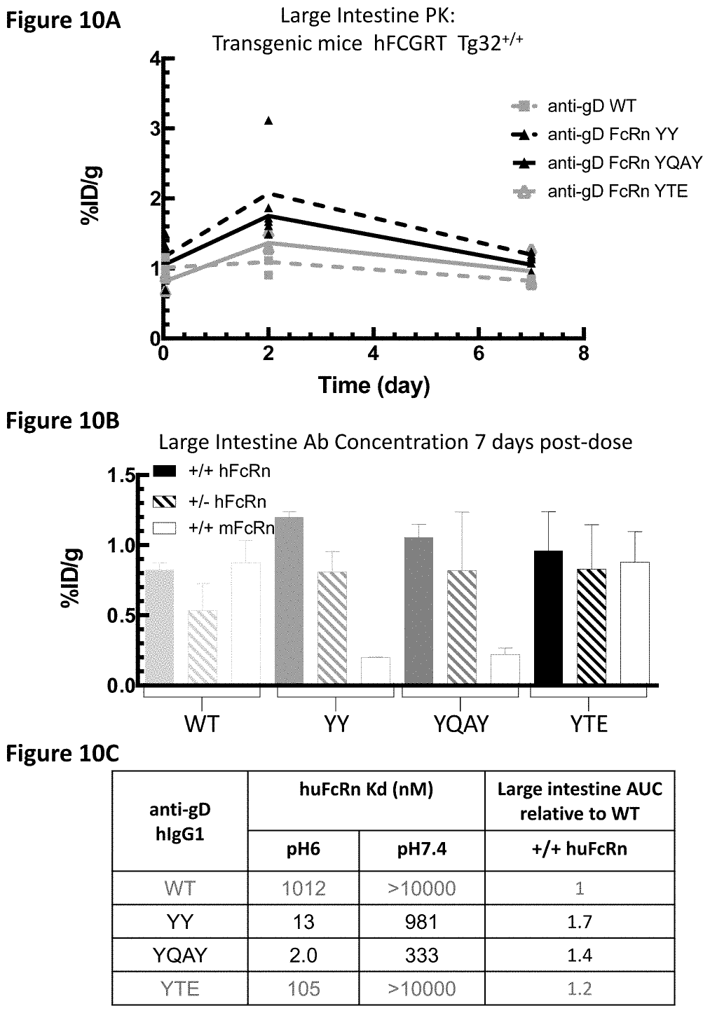

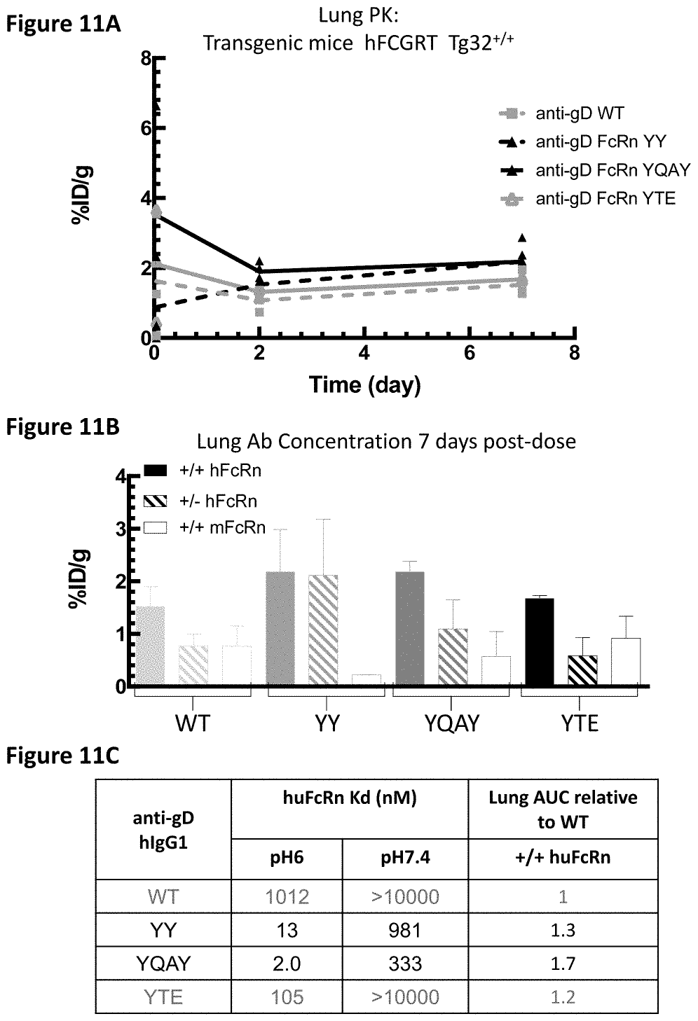

The pharmacokinetics of anti-gD-hIgG1 and the three Fc modified antibodies was also determined in other tissues, including liver, large intestine, and lung (FIGS. 9-11). These tissues also showed modest improvement in tissue penetration by the Fc modified antibodies, although the improvement is less than the improvement seen in brain. Without intending to be bound by any particular theory, it may be that these tissues have lower levels of FcRn or comprise vasculature with fenestrated capillaries, which may allow for some level of antibody penetration via diffusion.

### Example 7—Pharmacokinetics and Pharmacodynamics of High Transcytosis IgG1 Variants in Cynomolgus Monkeys

Cynomolgus monkey PK/PD studies. For both studies, four male cynomolgus monkeys aged 3 to 5 years were used per experimental group.

In the first study, anti-BACE1 hIgG1 wild-type antibody, and anti-BACE1 hIgG1 antibodies with modified Fcs (anti-BACE1-YQAY, YEY, YPY) were administered at 50 mg/kg via an intravenous bolus injection into the saphenous vein at day 0. CSF and blood samples were collected at various time points from 7 days before dosing up to 29 days post dose. Samples were collected at the same time of the day.

In the second study, anti-BACE1 hIgG1 wild-type antibody, and anti-BACE1 hIgG1 antibodies with modified Fcs (anti-BACE1-YQAY, YY, YLYI, YIY) were administered at 50 mg/kg via an intravenous bolus injection into the saphenous vein. CSF and blood samples were collected at various time points from 7 days before dosing up to 7 days after dosing. At 2 and 7 days post-dose, brains of two animals were harvested after full body perfusion. Brain regions were sub-dissected and immediately frozen. Different brain regions were homogenized in 1% NP-40 (Cal-Biochem) in PBS containing Complete Mini EDTA-free protease inhibitor cocktail tablets (Roche Diagnostics). Homogenized brain samples were rotated at 4° C. for 1 hour before spinning at 14,000 rpm for 20 minutes. The supernatant was isolated for brain pharmacokinetics and pharmacodynamics (sAPPβ/α) analysis.

Pharmacokinetics assays. Total antibody concentrations in monkey serum were measured using a LCMS method with affinity capture by Pure Proteome Protein A Magnetic Beads (Millipore) followed by denaturation, reduction, alkylation and trypsin digestion. A signature peptide from the Fc region was selected as the surrogate for the quantification of the total antibody concentration. The MQC of the assay was 60 ng/mL. Total antibody concentrations of CSF and brain samples were measured using an ELISA method with monkey-adsorbed sheep anti human IgG polyclonal antibody (Binding Site) as coat and a monkey adsorbed goat anti-human IgG antibody conjugated to HRP (Bethyl) as detection. The assay had an MQC value of 1.6 ng/ml in CSF and brain.

Pharmacodynamics assays. sAPPβ/α ratio. CSF and brain concentrations of sAPPα and sAPPβ were determined with a sAPPα/sAPPβ multiplex ECL assay. The anti-Abeta monoclonal antibody 6E10 was used to capture sAPPα, and an antibody directed against amino acids 591 to 596 of APP was used to capture APPβ. Both analytes were detected with an antibody directed against the N-terminus of APP. CSF was thawed on ice and then diluted 1:10 into 1% BSA in TBS-Tween 20. The assay had LLOQ values of 0.05 and 0.03 ng/ml for sAPPα and sAPPβ, respectively.

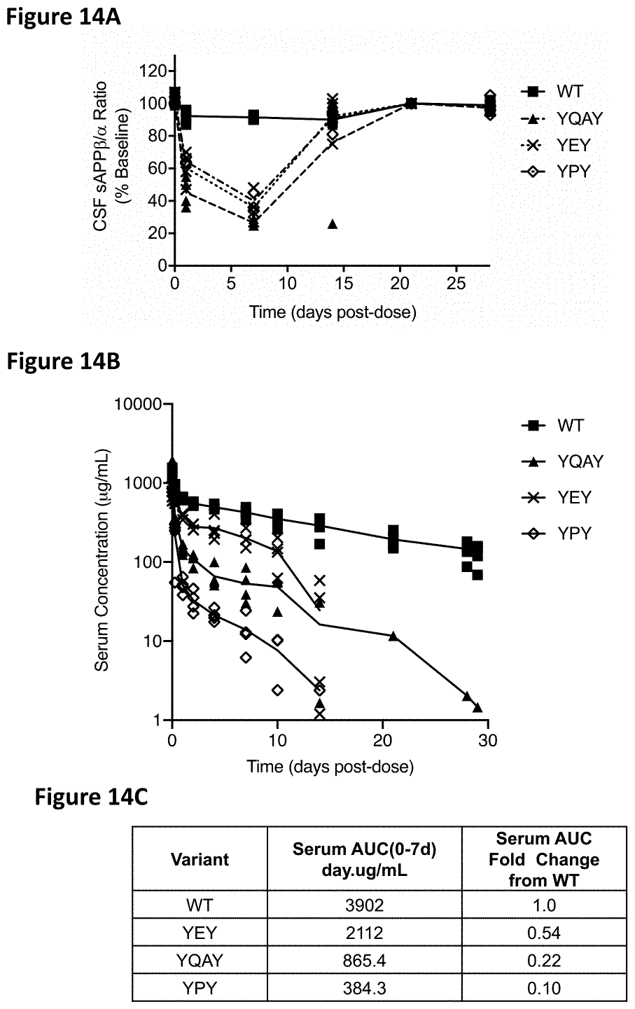

Results. As shown in FIG. 14A, in the first study, the anti-BACE1 antibodies with modified Fcs showed a significant decrease in cerebrospinal fluid (CSF) sAPPβ/α ratio compared to anti-BACE1 hIgG1 wild-type antibody. As shown in FIGS. 14B and 14C, FcRn affinity at neutral pH appeared to correlate with increased clearance of the antibody from serum, with anti-BACE1 hIgG1 wild-type antibody being cleared more slowly than any of the antibodies with modified Fcs.

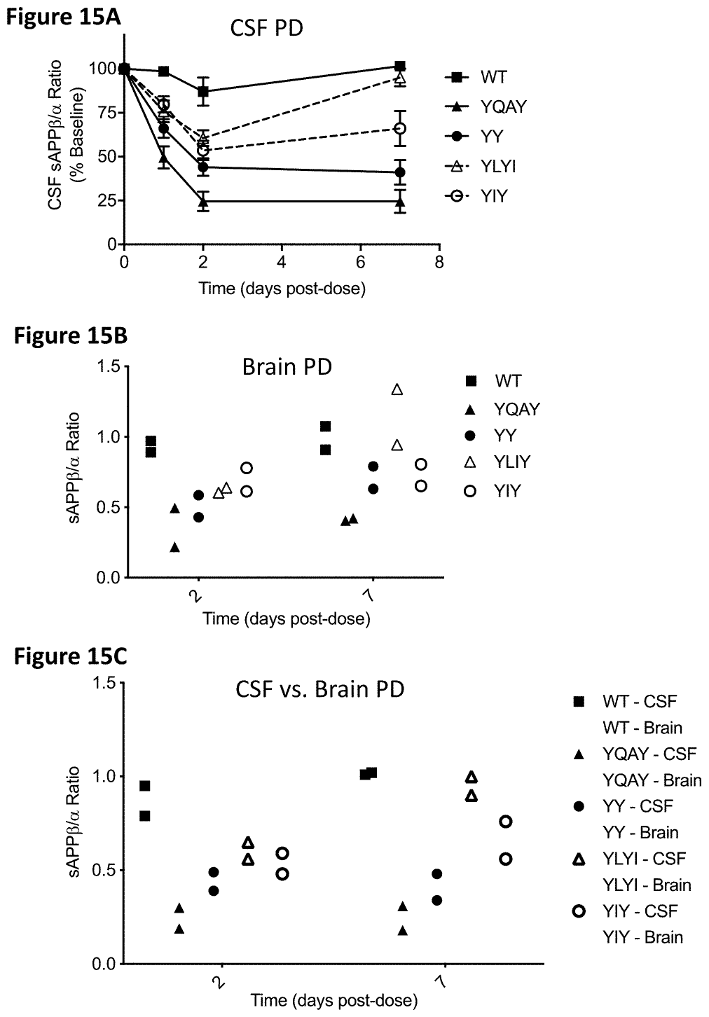

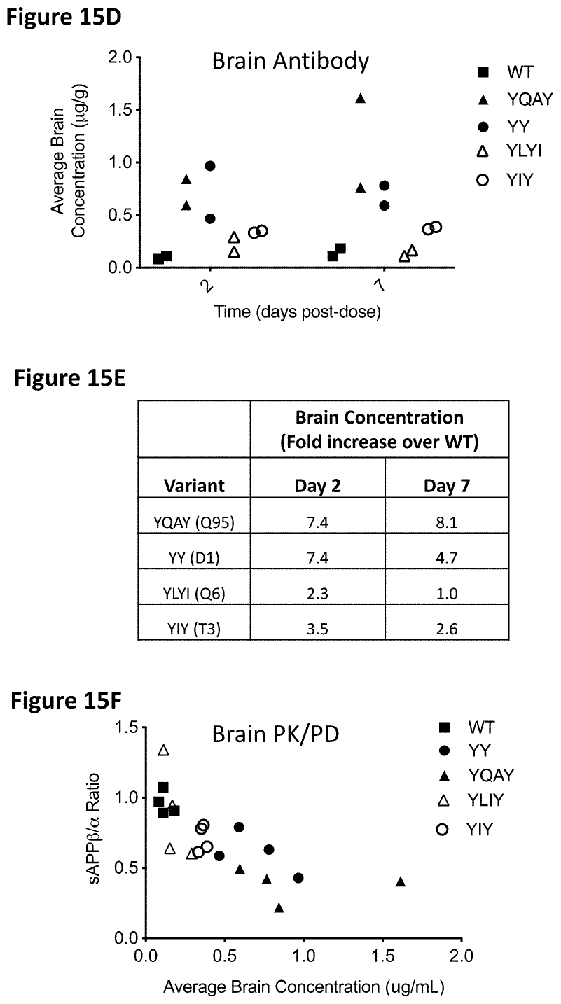

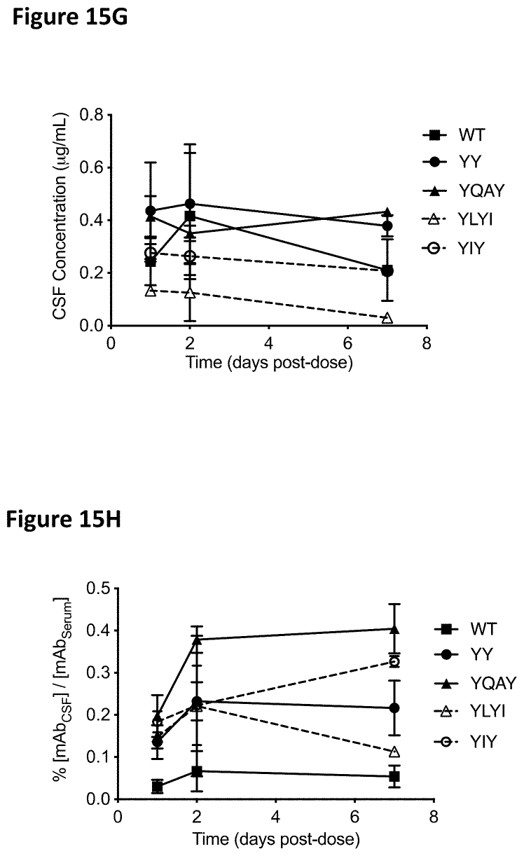

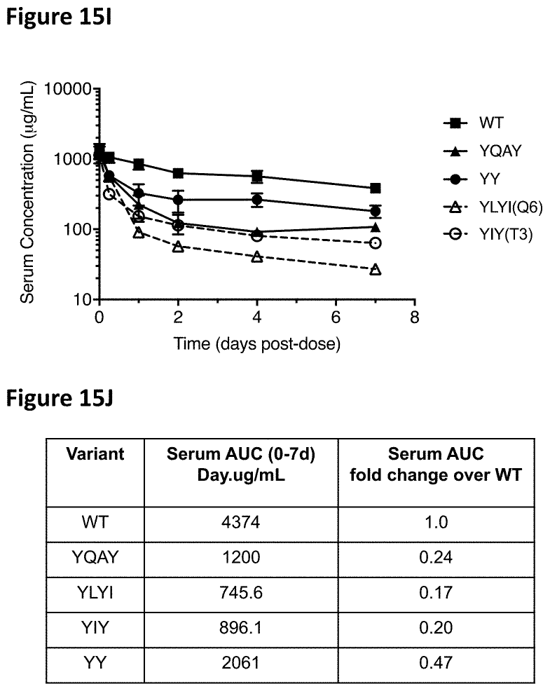

As shown in FIG. 15A, in the second study, the anti-BACE1 antibodies with modified Fcs showed a significant decrease in CSF sAPPβ/α ratio compared to anti-BACE1 hIgG1 wild-type antibody. As shown in FIG. 15B the anti-BACE1 antibodies with modified Fcs showed a substantial decrease in brain sAPPβ/α ratio compared to anti-BACE1 hIgG1 wild-type antibody. FIG. 15C shows a comparison of the CSF sAPPβ/α ratio and brain sAPPβ/α ratio for each of the antibodies. CSF and brain showed similar PD effects with the various antibodies.

As shown in 15D, all of the anti-BACE1 antibodies with modified Fcs were present at higher concentrations in brain (average of cortex and hippocampus values) at day 2 than the anti-BACE1 hIgG1 wild-type antibody, and the anti-BACE1 antibodies with YQAY, YY, and YIY Fc modifications had higher brain concentrations at day 7 than the anti-BACE1 hIgG1 wild-type antibody. The fold-improvement in antibody concentration at day 2 was 7.4, and at day 7 was 4.7 for anti-BACE1 hIgG1 YY. The fold-improvement in antibody concentration at day 2 was 7.4, and at day 7 was 8.1 for anti-BACE1 hIgG1 YQAY (FIG. 15E).

FIG. 15F shows the correlation between brain sAPPβ/α ratio and brain antibody concentration, which demonstrates that higher levels of anti-BACE1 antibody is the brain result in lower sAPPβ/α ratios and a stronger PD response.

FIG. 15G shows average CSF concentration of anti-BACE1 antibodies with wild-type or modified hIgG1 Fcs. Anti-BACE1 antibodies with hIgG1 Fcs comprising YY and YQAY modifications showed greater CSF concentrations compared to anti-BACE1 antibody with a hIgG1 wild-type Fc at different time points. FIG. 15H shows the ratio of the concentration of anti-BACE1 antibody in the CSF to the concentration of anti-BACE1 antibody in the serum. All of the modified Fcs, YY, YQAY, YLYI, and YIY, resulted in an increase in the proportion of anti-BACE1 antibody in the CSF. As shown in FIGS. 15I and 15J, the anti-BACE1 antibodies with modified Fcs were cleared more quickly from serum than anti-BACE1 hIgG1 wild-type antibody.

### Example 8—Assessing Anti-Abeta Antibody Target Engagement in PS2APP Transgenic Mice

As discussed in Example 2, modified Fc comprising M252Y/S254T/T256E (YTE) have improved binding to mFcRn at both pH 7.4 and pH 6.0, but improved hFcRn only at pH 6.0. In wild-type mice, anti-BACE1-hIgG1-YTE demonstrates improved brain uptake compared to anti-BACE1-hIgG1. To determine if the improved brain uptake is observed with antibodies that bind to other brain antigens, an anti-Abeta-hIgG4-YTE antibody was administered to PS2APP mice, which co-express human APP (hAPP) with the Swedish mutation K670N/M671L and human presenilin 2 with the N141I mutation, driven by Thy1 and PrP promoters, respectively. See, e.g., Richards et al., J. Neurosci. 23, 8989-9003 (2003). These mice accumulate oligomeric and fibrillar amyloid deposits in the brain, including amyloid plaques, and thus target engagement of anti-Abeta antibodies can be assessed. Generally, following administration of an anti-Abeta antibody, binding of the antibody is observed along the periphery of the amyloid plaques and in the mossy fiber hippocampal tract. This specific staining pattern is not observed with an isotype-matched control antibody. (Data not shown; see, e.g., Meilandt, W. J., et al. Characterization of the selective in vitro and in vivo binding properties of crenezumab to oligomeric Aβ. Alz Res Therapy 11, 97 (2019) doi:10.1186/s13195-019-0553-5.)

In vivo dosing. Transgenic PS2APP or nontransgenic (Ntg) littermates were randomized into treatment groups and received a single intravenous (i.v.) dose of either anti-Abeta hIgG4 or anti-Abeta hIgG4-YTE (20, 40, 80, or 120 mg/kg). Antibodies were diluted in platform buffer (20 mM histidine, 240 mM sucrose; pH 5.5, 0.02% Tween 20) and were injected at a volume of 5 ml/kg. Five days after dosing, the animals were sacrificed and terminal plasma was collected via cardiac puncture prior to perfusion with phosphate-buffered saline (PBS); the right hemibrain was removed and drop-fixed in 4% paraformaldehyde. From the left hemibrain, the hippocampus, cortex, and cerebellum were dissected, weighed, and stored at −80° C.

Immunohistochemistry. The right hemibrain was drop-fixed in 4% paraformaldehyde for 48 h and then transferred to 30% sucrose in PBS. Free-floating sagittal cryosections (35 μm) of mouse brain were washed in PBS and then PBS-Triton X100 (PBST, 0.1%) and then blocked in PBST (0.3%) with 5% bovine serum albumin (BSA) and incubated overnight with primary antibodies diluted in 1% BSA in PB ST (0.3%) at 4° C. Goat anti-human IgG-Alexa594 (or Alexa555, 1:100-1:500; Thermo-Fisher, Waltham, Mass.) was used to localize the administered human antibody. Plaques were detected using the AP fluorescent marker methoxy-X04.

Fluorescent microscopy. Whole slide images are captured at 20x using a Pannoramic 250 (3D Histech, Hungary) equipped with PCO.edge camera (Kelheim, Germany), Lumencor Spectra X (Beaverton, Oreg.), and Semrock filters (Rochester, N.Y.) optimized for 4′6-diamidino-2-phenylindole, dihydrochloride (DAPI), tetramethylrhodamine isothiocyanate (TRITC), and cyanine 5 (Cy5) fluorophores. Ideal exposure for each channel is determined based on samples with the brightest intensity and is set for the whole set of slides to run as a batch. Images were also captured at 20x using a Leica DM5500B light microscope using Leica Application Suite Advanced Florescence software (LAS AF4.0). Confocal images were taken using a 20x, or 40x oil objective on a Zeiss LSM800 confocal laser scanning microscope using the Zen2.3 software. Quantification of mossy fiber staining was performed by measuring integrated density from two to four sections per animal using ImageJ (NIH).

In vivo antibody pharmacokinetics (PK) measurements. Cerebellum samples were weighed and homogenized in 300 μl of 1% NP-40 (with Roche complete ETDA-free protease inhibitor cocktail) using a Qiagen TissueLyser II (2×3 min at 30 Hz). Samples were then placed on ice for 20 minutes and then centrifuged at 20,000×g for 20 minutes. Supernatant was collected and stored at −80° C. until used for PK assay. Antibody concentrations in mouse plasma and brain samples were measured using ELISA. NUNC 384-well Maxisorp immunoplates (Neptune, N.J., USA) were coated with F(ab′)2 fragment of sheep anti-human IgG, Fc fragment specific-polyclonal antibody (Jackson ImmunoResearch, West Grove, Pa., USA) overnight at 4° C. Plates were then blocked with PBS containing 0.5% BSA for 1 h at room temperature. Each antibody (anti-Abeta hIgG4 or anti-Abeta hIgG4-YTE) was used as a standard to quantify the respective antibody concentrations. After the plates were washed with PBS containing 0.05% Tween 20 using a microplate washer (Bio-Tek Instruments, Inc., Winooski, Vt.), standards and samples diluted in PBS containing 0.5% BSA, 0.35 M sodium chloride (NaCl), 0.25% 3-[(3-Cholamidopropyl)dimethylammonio]-1-propanesulfonate hydrate (CHAPS), 5 mM EDTA, 0.05% Tween 20, and 15 ppm Proclin were incubated on plates for 2 h at room temperature with mild agitation. Bound antibody was detected with horseradish peroxidase-conjugated F(ab′)2 goat anti-human IgG, Fc-fragment-specific polyclonal antibody (Jackson ImmunoResearch). Finally, plates were developed using the substrate 3,3′,5,5′-tetramethyl benzidine (KPL, Inc., Gaithersburg, Md., USA). Absorbance was measured at a wavelength of 450 nm with a reference of 630 nm on a Multiskan Ascent reader (Thermo Scientific, Hudson, N.H., USA). Concentrations were determined from the standard curve using a four-parameter nonlinear regression program. The assay had lower limit of quantitation values of 13.7 ng/ml in plasma and 1.37 ng/ml in brain.

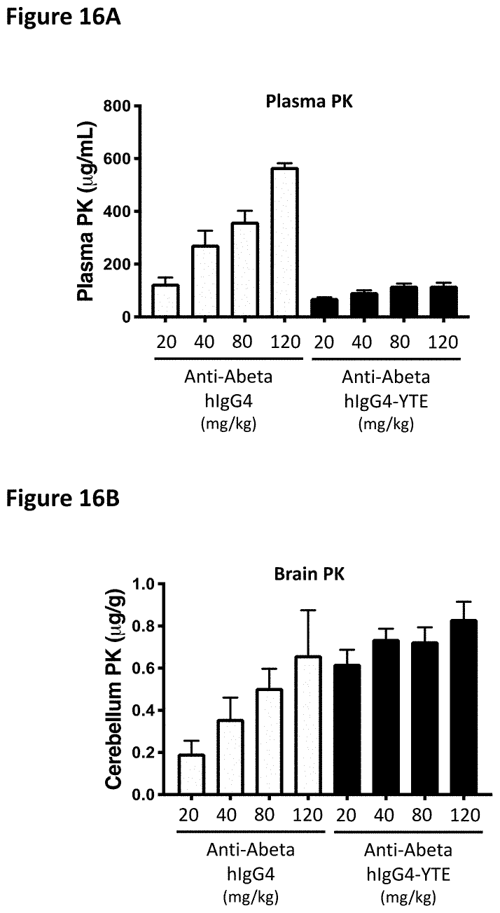

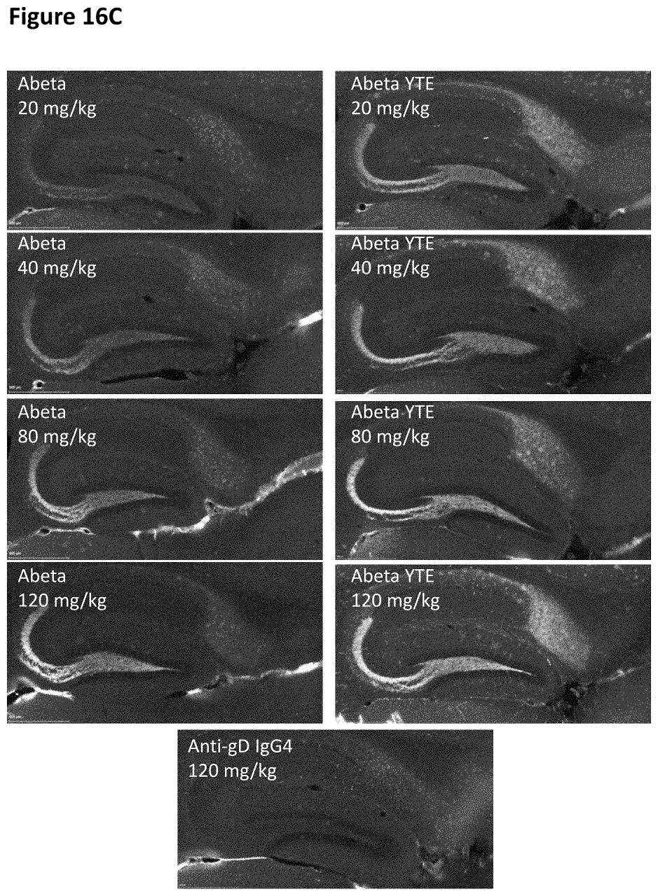

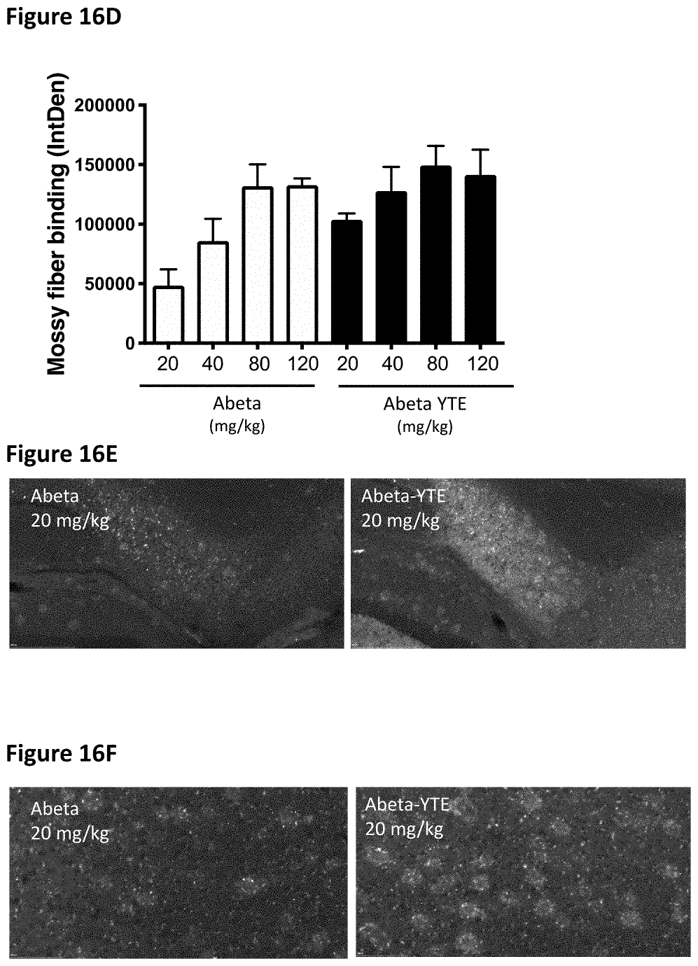

Results. As shown in FIGS. 16A-16B, a dose-dependent increase in plasma PK and brain PK was observed for anti-Abeta hIgG4, while anti-Abeta hIgG4-YTE showed lower serum exposure and enhanced brain uptake at all doses. The brain:plasma ratio for anti-Abeta hIgG4 was about 0.15%, and for anti-Abeta hIgG4 YTE was about 0.75%.

FIG. 16C shows in vivo target engagement as measured by antibody binding to the mossy fiber hippocampal tract. Anti-Abeta hIgG4 YTE shows significant binding following administration at 20 mg/kg, and increased binding as the dose is increased. Anti-Abeta hIgG4, in contrast, shows much lower staining compared to anti-Abeta hIgG4 YTE following administration at 20 mg/kg and 40 mg/kg. FIG. 16D is a bar graph showing the staining levels from FIG. 16C.

FIGS. 16E and 16F show binding of ant-Abeta hIgG4 and anti-Abeta hIgG4 YTE to the periphery of amyloid plaques in the subiculum (16E) and prefrontal cortex (16F) following administration at 20 mg/kg. The level of binding of anti-Abeta hIgG4 YTE to amyloid plaques in these brain regions is much greater than the binding observed for anti-Abeta hIgG4.

### Example 9—Pharmacokinetics of High Transcytosis IgG4 Variants in Cynomolgus Monkeys

Cynomolgus monkey PK studies. Four male cynomolgus monkeys aged 3 to 5 years were used per experimental group. Anti-Abeta hIgG4 wild-type antibody, and anti-Abeta hIgG4 antibodies with modified Fcs (anti-Abeta-YQAY, YEY, YY) were administered at 50 mg/kg via an intravenous bolus injection into the saphenous vein at day 0. The binding affinities for each of the antibodies for cyno FcRn at pH 7.4 and pH 6.0 are shown in Table 9. The affinities shown were measured at 25° C.; the affinities at 37° C. were similar (data not shown).

TABLE 9
Anti-Abeta hIgG4 antibody affinities for FcRn

| Fc modification | K_{D} pH 7.4 (nM) | K_{D} pH 6.0 (nM) |
|---|---|---|
| Wild-type | >2530 | >253 |
| YEY | 369 | 10.1 |
| YQAY | 215 | 8.2 |
| YY | 571 | 11.5 |

CSF and blood samples were collected at various time points from 7 days before dosing up to 7 days post dose. Samples were collected at the same time of the day. At 2 and 7 days post-dose, brains of two animals were harvested after full body perfusion. Brain regions were sub-dissected and immediately frozen. Different brain regions were homogenized in 1% NP-40 (Cal-Biochem) in PBS containing Complete Mini EDTA-free protease inhibitor cocktail tablets (Roche Diagnostics). Homogenized brain samples were rotated at 4° C. for 1 hour before spinning at 14,000 rpm for 20 minutes. The supernatant was isolated for brain pharmacokinetics analysis.

Pharmacokinetics assays. Total antibody concentrations in monkey serum, CSF, and brain samples were measured using an ELISA method with monkey-adsorbed sheep anti human IgG polyclonal antibody (Binding Site) as coat and a monkey adsorbed goat anti-human IgG antibody conjugated to HRP (B ethyl) as detection. The assay had an MQC value of 1.6 ng/ml in CSF and brain.

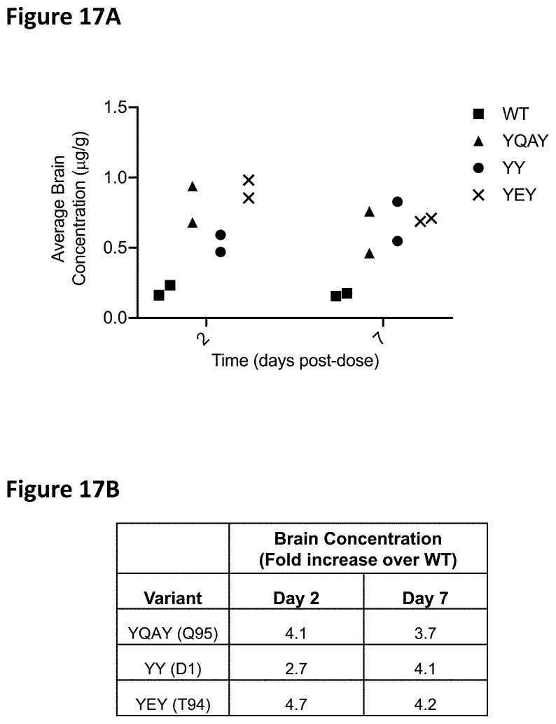

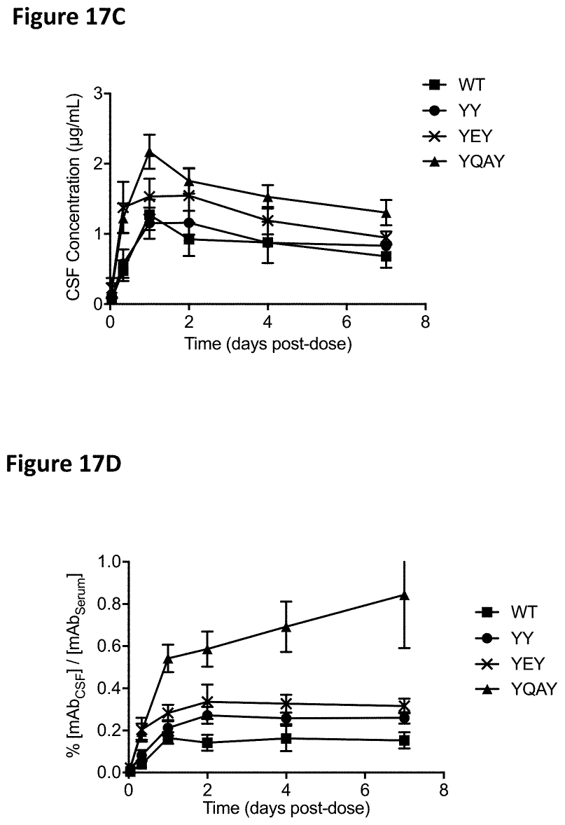

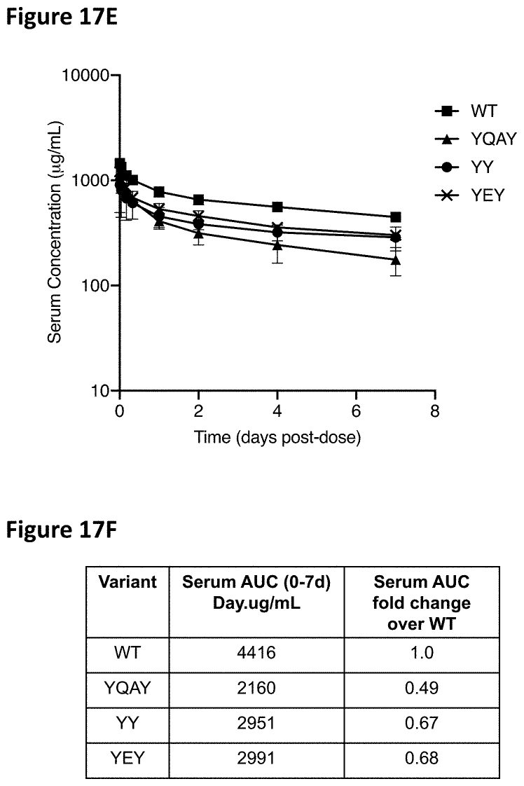

Results. FIG. 17A shows average brain concentration of the anti-Abeta antibodies at day 2 and day 7. FIG. 17B summarizes the fold improvement in brain concentration observed with each modified hIgG4 Fc. Anti-Abeta antibodies with modified Fcs showed 2.7- to 4.7-fold improvement in brain uptake compared to wild type Fc at day 2, and 3.7- to 4.2-fold improvement at day 7.

FIG. 17C shows average CSF concentration of anti-Abeta antibodies with wild-type or modified hIgG4 Fcs. Ant-Abeta antibodies with hIgG4 Fcs comprising YEY and YQAY modifications showed greater CSF concentrations compared to anti-Abeta antibody with a hIgG4 wild-type Fc. A small improvement was also observed with an Fc comprising YY modifications. FIG. 17D shows the ratio of the concentration of anti-Abeta antibody in the CSF to the concentration of anti-Abeta antibody in the serum. All of the modified Fcs, YY, YEY, and YQAY, resulted in an increase in the proportion of anti-Abeta antibody in the CSF. As shown in FIGS. 17E and 17F, the anti-Abeta antibodies with modified Fcs were cleared more quickly from serum than anti-Abeta hIgG4 wild-type antibody.

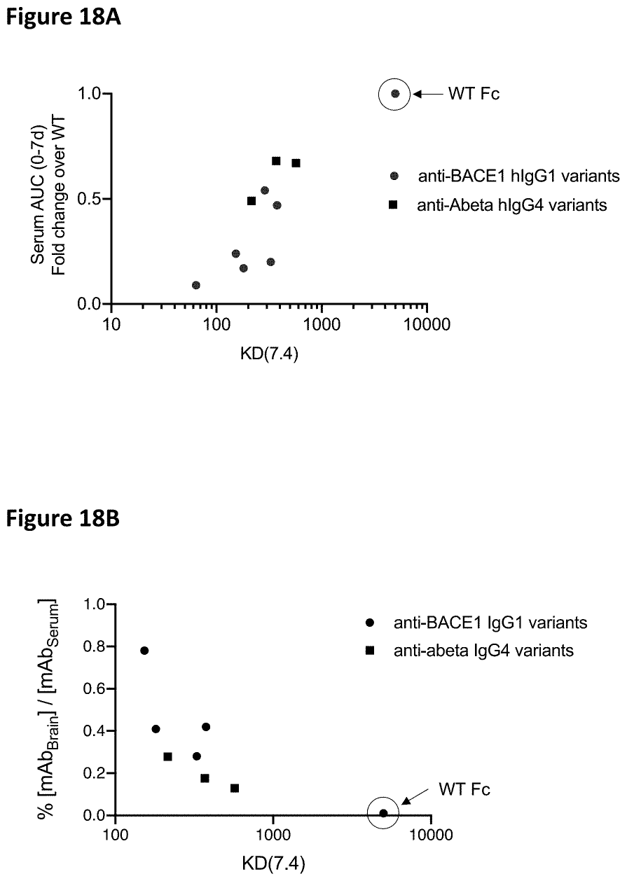

FIG. 18A shows a correlation of serum exposure in the cyno to affinity of the Fc for hFcRn at pH7.4 (K_{D} (7.4)). Both anti-BACE1 hIgG1 (circles) and anti-Abeta IgG4 (squares) Fc modification variants are shown on the plot. The WT Fc is circled and labeled.

FIG. 18B shows a correlation of antibody partitioning to brain (% [mAb_{Brain}]/[mAb_{Serum}]) in the cyno to affinity of the Fc for hFcRn at pH7.4 (K_{D} (7.4)). Both anti-BACE1 hIgG1 (circles) and anti-Abeta IgG4 (squares) Fc modification variants are shown on the plot. The WT Fc is circled and labeled.

In summary, the results demonstrate that modified Fcs with increased affinity for FcRn at pH7.4 improve brain exposure in cynomolgus monkeys in both an hIgG1 and hIgG4 context. Improvement of CSF exposure was also observed, particularly in comparison to serum exposure. Without intending to be bound by any particular theory, increased affinity for FcRn at pH7.4 may increase the fraction of IgG that is transcytosed rather than recycled or degraded, resulting in improved brain and CSF exposure. Alternatively, or in addition, increased affinity for FcRn on the cell surface at pH7.4 may increase the amount of IgG antibody that is internalized into cells and transcytosed, resulting in improved brain and CSF exposure.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### REFERENCES

Abbott, N. J., L. Ronnback and E. Hansson (2006). “Astrocyte-endothelial interactions at the blood-brain barrier.” Nat Rev Neurosci 7(1): 41-53.
Abuqayyas, L. and J. P. Balthasar (2013). “Investigation of the role of FcgammaR and FcRn in mAb distribution to the brain.” Mol Pharm 10(5): 1505-1513.
Atwal, J. K., Y. Chen, C. Chiu, D. L. Mortensen, W. J. Meilandt, Y. Liu, C. E. Heise, K. Hoyte, W. Luk, Y. Lu, K. Peng, P. Wu, L. Rouge, Y. Zhang, R. A. Lazarus, K. Scearce-Levie, W. Wang, Y. Wu, M. Tessier-Lavigne and R. J. Watts (2011). “A therapeutic antibody targeting BACE1 inhibits amyloid-beta production in vivo.” Sci Transl Med 3(84): 84ra43.
Borrok, M. J., Y. Wu, N. Beyaz, X. Q. Yu, V. Oganesyan, W. F. Dall'Acqua and P. Tsui (2015). “pH-dependent binding engineering reveals an FcRn affinity threshold that governs IgG recycling.” J Biol Chem 290(7): 4282-4290.
Challa, D. K., R. Velmurugan, R. J. Ober and E. Sally Ward (2014). “FcRn: from molecular interactions to regulation of IgG pharmacokinetics and functions.” Curr Top Microbiol Immunol 382: 249-272.
Chizzonite, R., T. Truitt, F. J. Podlaski, A. G. Wolitzky, P. M. Quinn, P. Nunes, A. S. Stern and M. K. Gately (1991). “IL-12: monoclonal antibodies specific for the 40-kDa subunit block receptor binding and biologic activity on activated human lymphoblasts.” J Immunol 147(5): 1548-1556.
Cooper, P. R., G. J. Ciambrone, C. M. Kliwinski, E. Maze, L. Johnson, Q. Li, Y. Feng and P. J. Hornby (2013). “Efflux of monoclonal antibodies from rat brain by neonatal Fc receptor, FcRn.” Brain Res 1534: 13-21.
Couch, J. A., Y. J. Yu, Y. Zhang, J. M. Tarrant, R. N. Fuji, W. J. Meilandt, H. Solanoy, R. K. Tong, K. Hoyte, W. Luk, Y. Lu, K. Gadkar, S. Prabhu, B. A. Ordonia, Q. Nguyen, Y. Lin, Z. Lin, M. Balazs, K. Scearce-Levie, J. A. Ernst, M. S. Dennis and R. J. Watts (2013). “Addressing safety liabilities of TfR bispecific antibodies that cross the blood-brain barrier.” Sci Transl Med 5(183): 183ra157, 181-112.
Dall'Acqua, W. F., P. A. Kiener and H. Wu (2006). “Properties of human IgG1s engineered for enhanced binding to the neonatal Fc receptor (FcRn).” J Biol Chem 281(33): 23514-23524.
Dall'Acqua, W. F., R. M. Woods, E. S. Ward, S. R. Palaszynski, N. K. Patel, Y. A. Brewah, H. Wu, P. A. Kiener and S. Langermann (2002). “Increasing the affinity of a human IgG1 for the neonatal Fc receptor: biological consequences.” J Immunol 169(9): 5171-5180.
Datta-Mannan, A., D. R. Witcher, Y. Tang, J. Watkins and V. J. Wroblewski (2007). “Monoclonal antibody clearance. Impact of modulating the interaction of IgG with the neonatal Fc receptor.” J Biol Chem 282(3): 1709-1717.
Eaton, D. L., W. I. Wood, D. Eaton, P. E. Hass, P. Hollingshead, K. Wion, J. Mather, R. M. Lawn, G. A. Vehar and C. Gorman (1986). “Construction and characterization of an active factor VIII variant lacking the central one-third of the molecule.” Biochemistry 25(26): 8343-8347.
Fishman, J. B., J. B. Rubin, J. V. Handrahan, J. R. Connor and R. E. Fine (1987). “Receptor-mediated transcytosis of transferrin across the blood-brain barrier.” J Neurosci Res 18(2): 299-304.
Fowler, D. M., C. L. Araya, S. J. Fleishman, E. H. Kellogg, J. J. Stephany, D. Baker and S. Fields (2010). “High-resolution mapping of protein sequence-function relationships.” Nat Methods 7(9): 741-746
Ghetie, V. and E. S. Ward (2002). “Transcytosis and catabolism of antibody.” Immunol Res 25(2): 97-113.
Ghosh, A. K., S. Gemma and J. Tang (2008). “beta-Secretase as a therapeutic target for Alzheimer's disease.” Neurotherapeutics 5(3): 399-408.
Hardy, J. and D. J. Selkoe (2002). “The amyloid hypothesis of Alzheimer's disease: progress and problems on the road to therapeutics.” Science 297(5580): 353-356.
Hinton, P. R., M. G. Johlfs, J. M. Xiong, K. Hanestad, K. C. Ong, C. Bullock, S. Keller, M. T. Tang, J. Y. Tso, M. Vasquez and N. Tsurushita (2004). “Engineered human IgG antibodies with longer serum half-lives in primates.” J Biol Chem 279(8): 6213-6216.
Hinton, P. R., J. M. Xiong, M. G. Johlfs, M. T. Tang, S. Keller and N. Tsurushita (2006). “An engineered human IgG1 antibody with longer serum half-life.” J Immunol 176(1): 346-356.
Igawa, T., A. Maeda, K. Haraya, T. Tachibana, Y. Iwayanagi, F. Mimoto, Y. Higuchi, S. Ishii, S. Tamba, N. Hironiwa, K. Nagano, T. Wakabayashi, H. Tsunoda and K. Hattori (2013). “Engineered monoclonal antibody with novel antigen-sweeping activity in vivo.” PLoS One 8(5): e63236.
Kuo, T. T. and V. G. Aveson (2011). “Neonatal Fc receptor and IgG-based therapeutics.” MAbs 3(5): 422-430.
Lo, M., H. S. Kim, R. K. Tong, T. W. Bainbridge, J. M. Vernes, Y. Zhang, Y. L. Lin, S. Chung, M. S. Dennis, Y. J. Zuchero, R. J. Watts, J. A. Couch, Y. G. Meng, J. K. Atwal, R. J. Brerski, C. Spiess and J. A. Ernst (2017). “Effector-attenuating Substitutions That Maintain Antibody Stability and Reduce Toxicity in Mice.” J Biol Chem 292(9): 3900-3908.
Mandelkow, E. M. and E. Mandelkow (2012). “Biochemistry and cell biology of tau protein in neurofibrillary degeneration.” Cold Spring Harb Perspect Med 2(7): a006247.
Morgan, M., S. Anders, M. Lawrence, P. Aboyoun, H. Pages and R. Gentleman (2009). “ShortRead: a bioconductor package for input, quality assessment and exploration of high-throughput sequence data.” Bioinformatics 25(19): 2607-2608.
Pardridge, W. M. (2016). “CSF, blood-brain barrier, and brain drug delivery.” Expert Opinion on Drug Delivery 13(7): 963-975.
Pardridge, W. M. (2016). “Re-engineering therapeutic antibodies for Alzheimer's disease as blood-brain barrier penetrating bi-specific antibodies.” Expert Opinion on Biological Therapy 16(12): 1455-1468.
Petkova, S. B., S. Akilesh, T. J. Sproule, G. J. Christianson, H. Al Khabbaz, A. C. Brown, L. G. Presta, Y. G. Meng and D. C. Roopenian (2006). “Enhanced half-life of genetically engineered human IgG1 antibodies in a humanized FcRn mouse model: potential application in humorally mediated autoimmune disease.” Int Immunol 18(12): 1759-1769.
Ponka, P. and C. N. Lok (1999). “The transferrin receptor: role in health and disease.” Int J Biochem Cell Biol 31(10): 1111-1137.
Roopenian, D. C. and S. Akilesh (2007). “FcRn: the neonatal Fc receptor comes of age.” Nat Rev Immunol 7(9): 715-725.
Spillantini, M. G., M. L. Schmidt, V. M. Lee, J. Q. Trojanowski, R. Jakes and M. Goedert (1997). “Alpha-synuclein in Lewy bodies.” Nature 388(6645): 839-840.
Thinakaran, G. and E. H. Koo (2008). “Amyloid precursor protein trafficking, processing, and function.” J Biol Chem 283(44): 29615-29619.
Vaccaro, C., J. Zhou, R. J. Ober and E. S. Ward (2005). “Engineering the Fc region of immunoglobulin G to modulate in vivo antibody levels.” Nat Biotechnol 23(10): 1283-1288.
Wong, A. W., T. K. Baginski and D. E. Reilly (2010). “Enhancement of DNA uptake in FUT8-deleted CHO cells for transient production of afucosylated antibodies.” Biotechnol Bioeng 106(5): 751-763.
Yeung, Y. A., M. K. Leabman, J. S. Marvin, J. Qiu, C. W. Adams, S. Lien, M. A. Starovasnik and H. B. Lowman (2009). “Engineering human IgG1 affinity to human neonatal Fc receptor: impact of affinity improvement on pharmacokinetics in primates.” J Immunol 182(12): 7663-7671.
Yu, Y. J., J. K. Atwal, Y. Zhang, R. K. Tong, K. R. Wildsmith, C. Tan, N. Bien-Ly, M. Hersom, J. A. Maloney, W. J. Meilandt, D. Bumbaca, K. Gadkar, K. Hoyte, W. Luk, Y. Lu, J. A. Ernst, K. Scearce-Levie, J. A. Couch, M. S. Dennis and R. J. Watts (2014). “Therapeutic bispecific antibodies cross the blood-brain barrier in nonhuman primates.” Sci Transl Med 6(261): 261ra154.
Yu, Y. J., Y. Zhang, M. Kenrick, K. Hoyte, W. Luk, Y. Lu, J. Atwal, J. M. Elliott, S. Prabhu, R. J. Watts and M. S. Dennis (2011). “Boosting brain uptake of a therapeutic antibody by reducing its affinity for a transcytosis target.” Sci Transl Med 3(84): 84ra44.
Zalevsky, J., A. K. Chamberlain, H. M. Horton, S. Karki, I. W. Leung, T. J. Sproule, G. A. Lazar, D. C. Roopenian and J. R. Desjarlais (2010). “Enhanced antibody half-life improves in vivo activity.” Nat Biotechnol 28(2): 157-159.

TABLE OF SEQUENCES

| SEQ | | |
|---|---|---|
| ID | | |
| NO | Description | Sequence |
| 1 | wild-type hIgG1 Fc, | PAPELLGGPS VFLFPPKPKD TLMISRTPEV TCVVVDVSHE |
| | including common | DPEVKFNWYV DGVEVHNAKT KPREEQYNST YRVVSVLTVL |
| | polymorphisms | HQDWLNGKEY KCKVSNKALP APIEKTISKA KGQPREPQVY |
| | | TLPPSR(D/E)E(L/M)T KNQVSLTCLV KGFYPSDIAV |
| | | EWESNGQPEN NYKTTPPVLD SDGSFFLYSK LTVDKSRWQQ |
| | | GNVFSCSVMH E(A/G)LHNHYTQK SLSLSPGK |
| 2 | wild-type hIgG2 Fc, | PAPPVAGPSV FLFPPKPKDT LMISRTPEVT CVVVDVSHED |
| | including common | PEVQFNWYVD G(V/M)EVHNAKTK PREEQFNSTF |
| | polymorphisms | RVVSVLTVVH QDWLNGKEYK CKVSNKGLPA PIEKTISKTK |
| | | GQPREPQVYT LPPSREEMTK NQVSLTCLVK GFYPSDIAVE |
| | | WESNGQPENN YKTTPPMLDS DGSFFLYSKL TVDKSRWQQG |
| | | NVFSCSVMHE ALHNHYTQKS LSLSPGK |
| 3 | wild-type hIgG3 Fc | PAPELLGGPS VFLFPPKPKD TLMISRTPEV TCVVVDVSHE |
| | | DPEVQFKWYV DGVEVHNAKT KPREEQYNST FRVVSVLTVL |
| | | HQDWLNGKEY KCKVSNKALP APIEKTISKT KGQPREPQVY |
| | | TLPPSREEMT KNQVSLTCLV KGFYPSDIAV EWESSGQPEN |
| | | NYNTTPPMLD SDGSFFLYSK LTVDKSRWQQ GNIFSCSVMH |
| | | EALHNRFTQK SLSLSPGK |
| 4 | wild-type hIgG4 Fc | PAPEFLGGPS VFLFPPKPKD TLMISRTPEV TCVVVDVSQE |
| | | DPEVQFNWYV DGVEVHNAKT KPREEQFNST YRVVSVLTVL |
| | | HQDWLNGKEY KCKVSNKGLP SSIEKTISKA KGQPREPQVY |
| | | TLPPSQEEMT KNQVSLTCLV KGFYPSDIAV EWESNGQPEN |
| | | NYKTTPPVLD SDGSFFLYSR LTVDKSRWQE GNVFSCSVMH |
| | | EALHNHYTQK SLSLSLGK |

1. A method of treating a neurological disorder comprising administering an antibody or an Fc conjugate comprising a modified IgG Fc to a subject in need thereof, wherein the antibody or the Fc conjugate is active in an in vitro transcytosis assay, wherein the in vitro transcytosis assay comprises cells that express FcRn.

2. The method of claim 1, wherein the neurological disorder is selected from a neuropathy disorder, a neurodegenerative disease, a brain disorder, cancer, an ocular disease disorder, a seizure disorder, a lysosomal storage disease, amyloidosis, a viral or microbial disease, ischemia, a behavioral disorder, and CNS inflammation.

3. (canceled)

4. (canceled)

5. A method of delivering an antibody or an Fc conjugate to the brain of a subject comprising administering to the subject an antibody comprising a modified IgG Fc to a subject in need thereof, wherein the antibody or the Fc conjugate is active in an in vitro transcytosis assay, wherein the in vitro transcytosis assay comprises cells that express FcRn.

6. A method of increasing brain exposure to an antibody or an Fc conjugate comprising administering to a subject an antibody comprising a modified IgG Fc to a subject in need thereof, wherein the antibody or the Fc conjugate is active in an in vitro transcytosis assay, wherein the in vitro transcytosis assay comprises cells that express FcRn.

7. A method of increasing transport of an antibody or an Fc conjugate across the blood brain barrier (BBB) comprising administering to a subject an antibody comprising a modified IgG Fc to a subject in need thereof, wherein the antibody or the Fc conjugate is active in an in vitro transcytosis assay, wherein the in vitro transcytosis assay comprises cells that express FcRn.

8. The method of claim 1, wherein the antibody or the Fc conjugate exhibits a transcytosis activity in the in vitro transcytosis assay of at least 50, at least 60, at least 70, at least 80, at least 90, or at least 100, when normalized to the same antibody or the same Fc conjugate comprising a wild-type IgG Fc.

9. (canceled)

10. (canceled)

11. (canceled)

12. The method of claim 1, wherein the antibody comprising the modified IgG Fc has a binding affinity for FcRn at pH 7.4 that is greater than the binding affinity of a reference antibody with an unmodified IgG Fc of the same species and isotype, and/or wherein the antibody comprising the modified IgG Fc has a binding affinity for FcRn at pH 6 that is greater than the binding affinity of a reference antibody with an unmodified IgG Fc of the same species and isotype.

13. (canceled)

14. (canceled)

15. (canceled)

16. The method of claim 1, wherein the ratio of the affinity of the antibody or the Fc conjugate comprising the modified IgG Fc for FcRn at pH 7.4 to the affinity of the antibody or the Fc conjugate comprising the modified IgG Fc for FcRn at pH 6 is at least 5, at least 10, at least 20, at least 50, or at least 100; or 5 to 200, 5 to 100, 10 to 200, 10 to 100, 20 to 100, or 20 to 200.

17. The method of claim 1, wherein the antibody or the Fc conjugate comprising the modified IgG Fc comprises one or more mutations selected from 252W, 252Y, 286E, 286Q, 307Q, 308P, 310A, 311A, 311I, 428L, 433K, 434F, 434W, 434Y, and 436I by EU numbering.

18. The method of claim 17, wherein the modified IgG Fc comprises 252Y and 434Y; or 252Y and 434Y and one or two additional mutations selected from 286E, 286Q, 307Q, 308P, 311A, 311I, 428L, 433K, and 436I; or 252Y, 434Y, 307Q, and 311A; or 252Y, 434Y, and 286E; or comprises a set of mutations selected from the set of mutations in Tables 4, 5, and 6.

19. (canceled)

20. (canceled)

21. (canceled)

22. (canceled)

23. (canceled)

24. The method of claim 1, wherein the antibody binds to a brain antigen.

25. The method of claim 24, wherein the antibody binds to a brain antigen selected from beta-secretase 1 (BACE1), amyloid beta (Abeta), epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), interleukin 6 receptor (IL6R), interleukin 1 beta (IL1β), caspase 6, triggering receptor expressed on myeloid cells 2 (TREM2), C1q, paired immunoglobin like type 2 receptor alpha (PILRA), CD33, interleukin 6 (IL6), tumor necrosis factor alpha (TNFα), tumor necrosis factor receptor superfamily member 1A (TNFR1), tumor necrosis factor receptor superfamily member 1B (TNFR2), and apolipoprotein J (ApoJ).

26. (canceled)

27. (canceled)

28. (canceled)

29. (canceled)

30. (canceled)

31. The method of claim 1, wherein the antibody is conjugated to an imaging agent or a neurological disorder drug or wherein the Fc conjugate comprises the modified IgG Fc conjugated to an imaging agent or a neurological disorder drug.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The method of claim 1, wherein the Fc conjugate comprises the modified IgG Fc fused to a therapeutic protein.

58. The method of claim 57, wherein the therapeutic protein is selected from a receptor extracellular domain and an enzyme.

59. The method of claim 58, wherein the receptor extracellular domain is selected from a TNF-R1 extracellular domain (ECD), a CTLA-4 ECD, and an IL-1R1 ECD; and/or wherein the enzyme is selected from alpha-L-iduronidase, iduronate-2-sulphatase, N-sulfatase, alpha-N-acetylglucosaminidase, N-acetyl-galactosamine-6-sulfatase, beta-galactosidase, arylsulphatase B, beta-glucuronidase, acid alpha-glucosidase, glucocerebrosidase, alpha-galactosidase A, hexosaminidase A, acid sphingomyelinase, beta-galactocerebrosidase, beta-galactosidase, arylsulfatase A, acid ceramidase, aspartoacylase, palmitoyl-protein thioesterase 1, and tripeptidyl amino peptidase 1.

60. (canceled)

61. (canceled)

62. (canceled)

63. (canceled)

64. An isolated antibody that binds to a brain antigen, wherein the antibody comprises a modified IgG Fc, wherein the antibody is active in an in vitro transcytosis assay, wherein the in vitro transcytosis assay comprises cells that express FcRn.

65. The isolated antibody of claim 64, wherein the antibody exhibits a transcytosis activity in the in vitro transcytosis assay of at least 50, at least 60, at least 70, at least 80, at least 90, or at least 100, when normalized to the same antibody comprising a wild-type IgG Fc.

66. (canceled)

67. (canceled)

68. (canceled)

69. The isolated antibody of claim 64, wherein the antibody comprising the modified IgG Fc has a binding affinity for FcRn at pH 7.4 that is greater than the binding affinity of a reference antibody with an unmodified IgG Fc of the same species and isotype, and/or wherein the antibody comprising the modified IgG Fc has a binding affinity for FcRn at pH 6 that is greater than the binding affinity of a reference antibody with an unmodified IgG Fc of the same species and isotype.

70. (canceled)

71. (canceled)

72. (canceled)

73. The isolated antibody of claim 64, wherein the ratio of the affinity of the antibody comprising the modified IgG Fc for FcRn at pH 7.4 to the affinity of the antibody comprising the modified IgG Fc for FcRn at pH 6 is at least 5, at least 10, at least 20, at least 50, or at least 100; or 5 to 200, 5 to 100, 10 to 200, 10 to 100, 20 to 100, or 20 to 200.

74. The isolated antibody of claim 64, wherein the antibody comprising the modified IgG Fc comprises one or more mutations selected from 252W, 252Y, 286E, 286Q, 307Q, 308P, 310A, 311A, 311I, 428L, 433K, 434F, 434W, 434Y, and 436I by EU numbering.

75. The isolated antibody of claim 74, wherein the modified IgG Fc comprises 252Y and 434Y; or 252Y and 434Y and one or two additional mutations selected from 286E, 286Q, 307Q, 308P, 311A, 311I, 428L, 433K, and 436L or 252Y, 434Y, 307Q, and 311A; or 252Y, 434Y, and 286E; or comprises a set of modifications selected from the set of mutations in Tables 4, 5, and 6.

76. (canceled)

77. (canceled)

78. (canceled)

79. (canceled)

80. (canceled)

81. The isolated antibody of claim 64, wherein the brain antigen is selected from beta-secretase 1 (BACE1), amyloid beta (Abeta), epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), interleukin 6 receptor (IL6R), interleukin 1 beta (IL1β), caspase 6, triggering receptor expressed on myeloid cells 2 (TREM2), C1q, paired immunoglobin like type 2 receptor alpha (PILRA), CD33, interleukin 6 (IL6), tumor necrosis factor alpha (TNFα), tumor necrosis factor receptor superfamily member 1A (TNFR1), tumor necrosis factor receptor superfamily member 1B (TNFR2), and apolipoprotein J (ApoJ).

82. (canceled)

83. (canceled)

84. (canceled)

85. (canceled)

86. (canceled)

87. The isolated antibody of claim 64, wherein the antibody is conjugated to an imaging agent or to a neurological disorder drug.

88. (canceled)

89. (canceled)

90. An Fc conjugate comprising a modified IgG Fc, wherein the Fc conjugate is active in an in vitro transcytosis assay, wherein the in vitro transcytosis assay comprises cells that express FcRn.

91. The Fc conjugate of claim 90, wherein the Fc conjugate exhibits a transcytosis activity in the in vitro transcytosis assay of at least 50, at least 60, at least 70, at least 80, at least 90, or at least 100, when normalized to the same Fc conjugate comprising a wild-type IgG Fc.

92. (canceled)

93. (canceled)

94. (canceled)

95. The Fc conjugate of claim 90, wherein the Fc conjugate comprising the modified IgG Fc has a binding affinity for FcRn at pH 7.4 that is greater than the binding affinity of a reference Fc conjugate with an unmodified IgG Fc of the same species and isotype, and/or wherein the Fc conjugate comprising the modified IgG Fc has a binding affinity for FcRn at pH 6 that is greater than the binding affinity of a reference Fc conjugate with an unmodified IgG Fc of the same species and isotype.

96. (canceled)

97. (canceled)

98. (canceled)

99. The Fc conjugate of claim 90, wherein the ratio of the affinity of the Fc conjugate comprising the modified IgG Fc for FcRn at pH 7.4 to the affinity of the Fc conjugate comprising the modified IgG Fc for FcRn at pH 6 is at least 5, at least 10, at least 20, at least 50, or at least 100; or 5 to 200, 5 to 100, 10 to 200, 10 to 100, 20 to 100, or 20 to 200.

100. The Fc conjugate of claim 90, wherein the Fc conjugate comprising the modified IgG Fc comprises one or more mutations selected from 252W, 252Y, 286E, 286Q, 307Q, 308P, 310A, 311A, 311I, 428L, 433K, 434F, 434W, 434Y, and 436I by EU numbering.

101. The Fc conjugate of claim 100, wherein the modified IgG Fc comprises 252Y and 434Y; or 252Y and 434Y and one or two additional mutations selected from 286E, 286Q, 307Q, 308P, 311A, 311I, 428L, 433K, and 436I; or 252Y, 434Y, 307Q, and 311A; or 252Y, 434Y, and 286E; or comprises a set of mutations selected from the sets of mutations in Tables 4, 5, and 6.

102. (canceled)

103. (canceled)

104. (canceled)

105. (canceled)

106. (canceled)

107. The Fc conjugate of claim 90, wherein the Fc conjugate comprises the modified IgG Fc fused to a therapeutic protein.

108. The Fc conjugate of claim 107, wherein the therapeutic protein is selected from a receptor extracellular domain and an enzyme.

109. The Fc conjugate of claim 108, wherein the receptor extracellular domain is selected from a TNF-R1 extracellular domain (ECD), a CTLA-4 ECD, and an IL-1R1 ECD; and/or wherein the enzyme is selected from alpha-L-iduronidase, iduronate-2-sulphatase, N-sulfatase, alpha-N-acetylglucosaminidase, N-acetyl-galactosamine-6-sulfatase, beta-galactosidase, arylsulphatase B, beta-glucuronidase, acid alpha-glucosidase, glucocerebrosidase, alpha-galactosidase A, hexosaminidase A, acid sphingomyelinase, beta-galactocerebrosidase, beta-galactosidase, arylsulfatase A, acid ceramidase, aspartoacylase, palmitoyl-protein thioesterase 1, and tripeptidyl amino peptidase 1.

110. (canceled)

111. (canceled)

112. The Fc conjugate of claim 90, wherein the Fc conjugate is conjugated to an imaging agent or to a neurological disorder drug.

113. (canceled)

114. (canceled)
